(19) United States
(12) Patent Application Publication
(10) Pub. No.: US 2014/0107170 A1
(43) Pub. Date: Apr. 17, 2014

(54) COMPOSITION COMPRISING ANGIOTENSIN II RECEPTOR ANTAGONIST AND ANTIOXIDANT FOR MAINTAINING AND/OR IMPROVING SKIN PROPERTIES

(71) Applicant: Miso Sabovic, Ljubljana (SI)

(72) Inventor: Miso Sabovic, Ljubljana (SI)

(21) Appl. No.: 14/115,667

(22) PCT Filed: May 4, 2012
(86) PCT No.: PCT/EP2012/058294

§ 371 Date: Nov. 5, 2013

(30) Foreign Application Priority Data
May 6, 2011 (SI) P-201100154
Jan. 24, 2012 (SI) P-201200021

(51) Int. Cl. A61K 31/4178; A61K 31/355; A61K 45/06; A61K 31/07; A61K 31/341; A61K 31/41

(52) U.S. Cl. CPC A61K 31/4178; A61K 31/341; A61K 31/41; A61K 45/06; A61K 31/07; A61K 31/355
USPC 514/381; 514/474

(57) ABSTRACT

The present invention relates to a topical composition comprising at least one angiotensin II receptor antagonist and at least one antioxidant.

### FIELD OF INVENTION

The present invention relates to a topical composition for application or administration to skin comprising at least one angiotensin II receptor antagonist and at least one antioxidant. The topical composition preferably maintains and/or improves the collagen structure of skin. In another aspect, the present invention also relates to a cosmetic topical composition for application to skin comprising at least one angiotensin II receptor antagonist and at least one antioxidant. Further, the present invention relates to the cosmetic use of the topical composition and a cosmetic method comprising contacting the topical composition with skin for improving or maintaining the biomechanical properties of skin, decreasing wrinkle depth, area and volume, protecting skin from oxidative stress, preventing or reducing skin roughness, maintaining or improving skin hydration, and/or maintaining or improving skin thickness. In yet another aspect, the present invention relates to a pharmaceutical topical composition for topical administration to skin comprising at least one angiotensin II receptor antagonist and at least one antioxidant. Further, the present invention relates to the pharmaceutical topical composition for use in treating or preventing skin irritation, treating or preventing skin sensations selected from the group consisting of stinging, burning tingling, itching, tickling, skin tightness or any combinations of the foregoing, treating or preventing skin redness, and/or treating neurodermitis.

### BACKGROUND OF INVENTION

Collagen is a group of naturally occurring proteins, eventually it is the most powerful protein in human system. The name collagen is derived from Greek and means healing or bonding. It makes up about 25% to 35% of the whole body protein content and its highest quantities occur in the skeletal system, the skin, organ of sight, hair, bones, nerves and vessels, the kidneys, liver, alimentary tract. So far scientists have discovered over twenty nine unique variants of collagen in human body.

Collagen is an unusual protein in all respects, its structure and metabolism involve a number of special features that are critical for deposition of normal collagen fibers. Collagen is one of the long, fibrous structural proteins. Tough bundles of collagen called collagen fibers are a major component of the extracellular matrix that supports most tissues and gives cells structure from the outside, but collagen is also found inside certain cells. Thus, collagen fibers form the collagen framework which is essential part of extracellular matrix of skin. Collagen has great tensile strength, that it is why collagen is the main component of fascia, cartilage, ligaments, tendons, bone and skin. Along with elastin it forms the framework that holds the skin together. Collagen is responsible for strength, elasticity, firmness, wrinkles, smoothness, resilience tone, roughness and hydration of skin. It strengthens blood vessels and plays a role in tissue development and remodeling. It is present in the cornea and lens of the eye in crystalline form. It is also used in cosmetic surgery and burns surgery.

Collagen undergoes the continuous physiological process of degradation that is simultaneously followed by its regeneration. This process is carried out practically every day since birth. The collagen matrix is partially degraded over time at diverse speeds in different individuals. Collagen degradation leads to decreased skin elasticity and firmness and to occurrence of wrinkles.

Angiotensin II receptor (AT-1 and AT-2 receptors) antagonists, also known as angiotensin receptor blockers (ARBs) or sartans, are a group of compounds which modulate the renin-angiotensin-aldosterone system. Their main therapeutic use is in hypertension (high blood pressure), diabetic nephropathy (kidney damage due to diabetes), congestive heart failure and prevention of cardiac remodeling after myocardial infarction. ARBs are receptor antagonists that block angiotensin receptors on bloods vessels and also in other tissues as arterial wall, heart muscle, kidney.

The role of angiotensin II receptor antagonist in increasing the formation of collagen in different tissues particularly unhealthy tissues is known from the state of the art. For example WO 02/40007 and WO 2005/049013 disclose the use of a pharmaceutical composition comprising an AT1-receptor antagonist or a pharmaceutically acceptable salt thereof in a combination with other active substances such as renin inhibitor or diuretic amiloride or triameterine or a pharmaceutical acceptable salt thereof for the prevention of or delay of progression of or treatment of increasing of formation of collagen.

WO 2005/072696 relates to a cosmetic method for improving and/or maintaining the skin tone comprising contacting the skin with a composition comprising at least one ACE inhibitor and/or angiotensin II receptor antagonist

WO 2006/105805 discloses the use of a topical composition comprising three or more active substances, for the preparation of a medicament for reducing or suppressing skin ageing and disorders associated with skin ageing and their symptoms such as wrinkles.

In accordance with the persistent trend towards skin care products for refreshing, smoothing and protecting the skin, there remains a need for cosmetic compositions which have improved and wide-ranging skin care properties.

Furthermore, in accordance with the problem that intrinsic (e.g. genetic predisposition) and extrinsic (e.g. exposure to UV radiation or contact with irritating or allergy-inducing substances) factors can cause skin irritation, there is a constant need for improved pharmaceutical compositions which are useful in the treatment of skin irritation.

### SUMMARY OF INVENTION

It is therefore an object of the present invention to provide cosmetic or pharmaceutical topical compositions, which have advantages over the prior art. In particular, the cosmetic topical compositions should have improved and wide-ranging skin care properties, and the pharmaceutical topical composition should be advantageous for the treatment of e.g. skin irritation, skin sensation, skin redness, or neurodermitis.

In a first aspect, the invention is directed to a topical composition comprising at least one angiotensin II receptor antagonist and at least one antioxidant.

It has been found that the topical compositions according to the invention have a positive influence on skin properties and skin characteristics, when topically applied to the skin already after a short time such as 14 days of application.

It has been found that “day-to-day” changes of skin properties in subjects at any age are addressed. In general, skin properties (i.e. their quality) depend on numerous factors. Of particular importance is the fact that aging influences skin properties throughout a long period, whereas other factors can induce/produce changes in skin properties in a short period. These short-term changes, i.e. “day-to-day” changes of skin properties are particularly addressed by the present invention.

Importantly, in these short-term changes of skin properties, the aging process typically does not have any impact at all. However, the following factors do have an impact:
a) environmental factors (e.g. pollution, climate, air-condition, natural irritants in the air),
b) habitual factors (e.g. stress, depression and mood changes, food, physical activity, hydration, sleeping)
c) hormonal factors (e.g. menstrual cycle, the levels of sex hormones, sympathetic over-activity)
d) genetic factors,
e) intrinsic capacity of skin for protection and regeneration.

Advantageously, the topical composition according to the present invention is suitable for defeating at least one of the above listed impacts a), b), c), d), or e), thereby improving the “day-to-day” skin properties and skin characteristics.

Furthermore, it has been found that the topical compositions according to the invention have a positive influence on the symptoms of irritated skin and/or neurodermitis, e.g. when administered to the skin. Furthermore, skin redness and skin sensations selected from the group consisting of stinging, burning, tingling, itching, tickling, skin tightness or any combinations of the foregoing can be treated.

It has also been found that the topical compositions according to the invention maintain or improve the collagen structure of skin.

In another aspect, the invention is directed to cosmetic topical composition, the use of said topical composition, and a cosmetic method comprising contacting said topical composition with skin for
a) maintaining or improving the biomechanical properties of skin,
b) decreasing wrinkle depth, wrinkle area and wrinkle volume,
c) protecting skin from oxidative stress,
d) preventing or reducing skin roughness,
e) maintaining or improving skin hydration, and/or
f) maintaining or improving skin thickness.

In another aspect, the invention is directed to a pharmaceutical topical composition for use in
a) treating or preventing skin irritation,
b) treating or preventing neurodermitis,
c) treating or preventing skin sensations selected from the group consisting of stinging, burning, tingling, itching, tickling, skin tightness or any combinations of the foregoing, and/or
d) treating or preventing skin redness.

In particular, the inventors of the present application found out that the topical composition according to the present invention provides beneficial effect in quality of healthy skin by substantially improving and/or maintaining characteristics of apparently healthy skin. Without being bound to theory, it is believed that optimal collagen structure improves and/or maintains for example the following characteristics of skin:

a) Biomechanical properties (skin fatigue, elasticity and firmness)

b) Wrinkle depth, area/volume

c) Roughness

d) Hydration

e) Anti-oxidant properties

f) Regenerative and protective properties.

The topical composition according to the present invention increases not only skin firmness, but at the same time it also increases skin elasticity and skin fatigue resistance. Overall, the topical composition induces a change in the biomechanical properties of the skin towards the firm-elastic optimum.

The topical composition for application or administration to skin according to the present invention significantly decreases wrinkle depth, area and volume in regular treatment.

The topical composition for application or administration to skin according to the present invention shows an efficacy against free radicals, i.e. treatment of the epidermal surface before exposure to UVA with the topical composition prevents the discoloration of β-carotene.

The topical composition for application or administration to skin according to the present invention has both a statistically significant regeneration and protection effect against the damaging properties of certain harmful compounds such as the surfactant SDS with regards to its effect on the barrier function of the skin (i.e. to accelerate recovery from barrier damage and to protect from barrier damage) and on induced skin irritations such as skin redness (i.e. to accelerate recovery from induced skin redness and to protect from induction of skin redness).

The topical composition for application or administration to skin according to the present invention is suitable for subjects of whatever age: young, middle-age or elderly since the structure of skin can be impaired due to several reasons independently on ages, as for example exposure to sun, acnes, pollution, influence of hormones, ageing etc.

The topical composition for application or administration to skin according to the present invention can be used for cosmetic or for pharmaceutical purposes.

### FIGURES

FIG. 1: Mean increase in skin firmness relative to initial conditions and untreated [%]

FIG. 2: Mean increase in skin elasticity relative to initial conditions and untreated [%]

FIG. 3: Mean decrease in wrinkle depth relative to initial conditions [%]

FIG. 4: Color index (inhibition of discoloration (oxidative stress) relative to initial conditions and untreated) [%]

FIG. 5a: Mean cutometer readings (skin firmness, F4)

FIG. 5b: Mean increase in skin firmness relative to initial conditions and to untreated [%]

FIG. 6a: Mean cutometer readings (skin elasticity, F3/F4)

FIG. 6b: Mean increase in skin elasticity relative to initial conditions and to untreated [%]

FIG. 7a: Mean cutometer readings (skin fatigue resistance, F2)

FIG. 7b: Mean increase in skin fatigue resistance relative to initial conditions and to untreated [%]

FIG. 8a: Mean PRIMOS readings for wrinkle depth analysis

FIG. 8b: Mean decrease in wrinkle depth relative to initial conditions [%]

FIG. 8c: Mean PRIMOS readings for wrinkle area/volume analysis

FIG. 8d: Mean decrease in wrinkle area/volume relative to initial conditions [%]

FIG. 9a: Mean chromameter readings (b*) for discoloration of carotene

FIG. 9b: Mean discoloration of carotene relative to initial conditions and to untreated [%]

FIG. 10a: Mean chromameter readings (a*) for skin redness 1 day, 2 days, 4 days and 7 days after SDS—regeneration (delta values) FIG. 10b: Mean chromameter readings (a*) for skin redness after 1 week SDS to start (end of regeneration)—protection (delta values)

FIG. 10c: Mean tewameter readings (g/(hm²)) for TEWL 1 day, 2 days, 4 days and 7 days after SDS—regeneration (delta values) FIG. 10d: Mean tewameter readings (g/(hm²)) for transepidermal water loss (g/(hm²)) after 1 week SDS to start (end of regeneration)—protection (delta values)

FIG. 11: Mean collagenoson ICU readings for skin thickness

FIG. 12: Dermal fibroblast survival under oxidative stress after preincubation in control medium, in medium with added valsartan alone, vitamins ACE alone or combination

FIG. 13: Envelope curve for determining biomechanical properties of skin

### DEFINITIONS

Angiotensin II receptor (AT-1 and AT-2 receptors) antagonists, also known as angiotensin receptor blockers (ARBs) or sartans, are a group of pharmaceuticals which modulate the renin-angiotensin-aldosterone system.

The term angiotensin II receptor antagonist as used in the present invention can include, but is not limited to, azilsartan, losartan, eprosartan, irbesartan, olmesartan, candesartan, valsartan and telmisartan and any cosmetically or pharmaceutically acceptable salts thereof, esters thereof and any combination of the foregoing. Preferably angiotensin II receptor antagonist can be selected from the group consisting of, but not limited to, azilsartan, losartan, telmisartan, olmesartan, candesartan and valsartan and any cosmetically or pharmaceutically acceptable salts, thereof, esters thereof and any combination of the foregoing, more preferably it can be selected from the group consisting of, but not limited to, losartan, telmisartan, azilsartan, candesartan and valsartan and any cosmetically or pharmaceutically acceptable salts thereof, esters thereof and any combination of the foregoing and even more preferably angiotensin II receptor antagonist is valsartan or losartan and any cosmetically or pharmaceutically acceptable salts thereof, esters thereof and any combination of the foregoing.

Moreover, the topical composition of the present invention can further comprise one or more further active substance such as for example, but not limited to, a diuretic, such as for example thiazide such as for example chlorothiazide, chlorthalidone, hydrochlorothiazide, hydroflumethiazide, indapamide, methyclothiazide, metolazone, polythiazide, preferably hydrochlorothiazide and indapamide; loop diuretic such as for example bumetanide, ethacrynic acid, furosemide, torsemide, preferably furosemide and torsemide; K+-sparing diuretic such as for example amioloride, eplerenone, spironolactone, triamterene, preferably eplerenone and spironolactone; and Ca-inhibitors such as for example acetazolamide, dichlorphenamide, methazolamide; more preferably hydrochlorothiazide; combination with calcium channel blockers such as for example dihydropiridine calcium channel blockers that can be selected from the group consisting of, but not limited to, amlodipine, aranidipine, azelnidipine, barnidipine, benidipine, felodipine, lacidipine, lercanidipine, manidipine, nicardipine, nifedipine, nilvadipine, nimodipine, nisoldipine, nitrendipine, pranidipine, preferably amlodipine, and any pharmaceutically or cosmetically acceptable salts or esters thereof and any combinations thereof.

Preferably, the angiotensin II receptor antagonists as used herein are selected from the group consisting of azilsartan, losartan, eprosartan, irbesartan, olmesartan, candesartan, valsartan and telmisartan and any pharmaceutically or cosmetically acceptable salts or esters thereof and any combinations of the foregoing.

An antioxidant is known as a chemical that can protect against damage for example to cells which are typically induced by free radicals. Preferably, the antioxidant neutralizes free radicals by accepting or donating an electron to eliminate an unpaired condition. Typically, this means that the antioxidant molecule becomes a free radical in the process of neutralizing a free radical molecule to a non-free radical molecule. The antioxidant will usually be a much less reactive free radical than the free radical neutralized. Therefore, an antioxidant inhibits the oxidation of other molecules. Oxidation is a chemical reaction that transfers electrons from a substance to an oxidizing agent. Oxidation reactions can produce free radicals. In turn, these radicals can start chain reactions that damage cells. Antioxidants may terminate oxidation chain reactions by removing free radical intermediates, and inhibit other oxidation reactions.

The term antioxidant as used in the present invention can include, but is not limited to, butylated hydroxyanisole, butylated hydroxytoluene, malic acid, ascorbyl palmitate, retinyl palmitate, sodium ascorbate, sodium metabisulphite, propyl gallate, beta-carotene, ascorbic acid, sodium ascorbyl phosphate, magnesium ascorbyl phosphate, ascorbic acid-2-glycoside, ascorbyl palmitate, ascorbyl stearate, α-lipoic acid, glutathione, coenzyme Q10, tocopherol, tocopherol acetate, retinol, retinol palmitate, genistein, quercetin, epigallocatechin, epigallocatechin gallate, gallocatechin gallate, sylibin, diosmetin, kaempferol, epicatechin, galangin, indolic acid, γ-linolenic acid, linoleic acid, chlorogenic acid, tocotrienol, astaxanthin, and any cosmetically or pharmaceutically acceptable salts thereof, any analogues thereof and any combination of the foregoing. Preferably, the antioxidant can be selected from the group consisting of, but not limited to, beta-carotene, retinyl palmitate, ascorbic acid, tocopherol, tocopherol acetate, sodium ascorbyl phosphate, coenzyme Q10, resveratrol, magnesium ascorbyl phosphate, ascorbic acid-2-glycoside, butylated hydroxyanisole, chlorogenic acid, epigallocatechin gallate, indolic acid, α-lipoic acid and any cosmetically or pharmaceutically acceptable salts thereof and/or any analogues thereof, more preferably beta-carotene, ascorbic acid, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole and any cosmetically or pharmaceutically acceptable salts thereof and/or any analogues thereof and even more preferably beta-carotene, ascorbic acid, tocopherol, tocopherol acetate, sodium ascorbyl phosphate, retinyl palmitate, coenzyme Q10 and resveratrol and any cosmetically or pharmaceutically acceptable salts thereof and/or any analogue thereof.

Preferred antioxidants according to the invention are selected from the group consisting of beta-carotene, ascorbic acid, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole and any pharmaceutically or cosmetically acceptable salts and/or any analogues thereof, and any combinations of the foregoing.

As used herein, the term “analogue” preferably covers precursors and derivatives such as amides, ethers, esters, and the like.

Several antioxidants are also known as vitamins. As used herein, the term “vitamin C” refers to ascorbic acid and any pharmaceutically or cosmetically acceptable salts or analogues thereof. Preferably, the term “vitamin C” refers to ascorbic acid, ascorbyl palmitate, sodium ascorbate, sodium ascorbyl phosphate, magnesium ascorbyl phosphate, ascorbic acid-2-glycoside, ascorbyl palmitate, ascorbyl stearate, and any cosmetically or pharmaceutically acceptable salts thereof, and any combination of the foregoing, and preferably to ascorbic acid.

As used herein, the term “Vitamin A” refers to beta-carotene and any pharmaceutically or cosmetically acceptable salts or analogues thereof, such as retinol, retinyl palmitate, retinol palmitate, and any cosmetically or pharmaceutically acceptable salts thereof, any analogues thereof or any combination of the foregoing, and preferably to beta-carotene.

As used herein, the term “Vitamin E” refers to tocopherol and any pharmaceutically or cosmetically acceptable salts or analogues thereof, preferably to tocopherol, tocopherol acetate, tocotrienol, and any cosmetically or pharmaceutically acceptable salts thereof, any analogues thereof and any combination of the foregoing, and preferably to tocopherol.

As used herein, “cosmetically acceptable salt” means that the salt is suitable for use in contact with tissues, such as the skin, e.g. human skin, without undue toxicity, incompatibility, instability, irritation, allergic response, and the like. Examples of cosmetically acceptable salts include inorganic and organic acid addition salts and basic salt. The cosmetically acceptable salts include, but are not limited to metal salts, such as sodium salt, potassium salt, caesium salt, and the like; alkaline earth metals, such as calcium salt, magnesium salt and the like, organic amine salts such as triethylamine salt, pyridine salt, picoline salt, ethanolamine salt, triethanolamine salt, dicyclohexylamine salt, N,N′-dibenzylethylenediamine salt and the like, inorganic acid salts, such as hydrochloride, hydrobromide, phosphate, sulphate and the like, organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like, and amino acid salts such as arginate, glutamate, and the like. Acid addition salts can be formed by mixing a solution of the particular compound of the present invention with a solution of a cosmetically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, and the like. Basic salts can be formed by mixing a solution of the particular compound of the present invention and a cosmetically acceptable non-toxic base such as sodium hydroxide, potassium hydroxide, choline hydroxide, sodium carbonate and the like.

The term “pharmaceutically acceptable salt” includes any and all non-toxic salts of the disclosed compounds. Examples of pharmaceutically acceptable salts include inorganic and organic acid addition salts and basic salt. The pharmaceutically acceptable salts include, but are not limited to metal salts, such as sodium salt, potassium salt, caesium salt, and the like; alkaline earth metals, such as calcium salt, magnesium salt and the like, organic amine salts such as triethylamine salt, pyridine salt, picoline salt, ethanolamine salt, triethanolamine salt, dicyclohexylamine salt, N,N′-dibenzylethylenediamine salt and the like, inorganic acid salts, such as hydrochloride, hydrobromide, phosphate, sulphate and the like, organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like, and amino acid salts such as arginate, glutamate, and the like. Acid addition salts can be formed by mixing a solution of the particular compound of the present invention with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, and the like. Basic salts can be formed by mixing a solution of the particular compound of the present invention and a pharmaceutically acceptable non-toxic base such as sodium hydroxide, potassium hydroxide, choline hydroxide, sodium carbonate and the like.

As used herein, the term “barrier compound” preferably refers to a compound, which shields skin from UV radiation, i.e. acts as a sunscreen. A barrier compound according to the invention is e.g. an UV filter, preferably an UVA or UVB filter. For example, inorganic particles, such as titanium dioxide or zinc oxide, may act as barrier compounds by forming a physical barrier, reflecting or scattering UV waves.

As used herein, “skin” preferably refers to animal or human skin. The term “skin” preferably includes the skin of the whole body. More preferably “skin” refers to human skin of the whole body, most preferably the skin of the face, neck, scalp, back hand and cleavage, particularly preferably of the face and neck. According to the invention, the term “skin” preferably covers all types of skin such as dry, oily or mixed type skin. Furthermore the skin may belong to subjects of all races and ethnicities and to subjects of all ages, e.g. young, middle-age or elderly subjects, in each case male or female.

The skin is preferably free of any skin diseases selected from the group consisting of psoriasis, pemphigus, dermatitis, vitiligo, rosacea, mucous ulcers and mucous abnormalities. Furthermore, the skin may be affected by extrinsic factors such as exposure to sun, acnes, and pollution, or influenced e.g. by hormones or aging. The skin may also be irritated, e.g. by extrinsic factors such as exposure to UV radiation or contact with irritating or allergy-inducing substances such as surfactants.

The term “apparently healthy skin” as used in the present invention means skin free of any skin diseases such as psoriasis, pemphigus, dermatitis, vitiligo, rosacea, mucous ulcers, mucous abnormalities. On the other hand said skin disease does not include the conditions/diseases as for example skin incisions acnes, burns and similar.

As used herein, the term “clinically healthy subjects” are subjects which do not have any diseases, i.e. chronic or acute diseases, in particular no skin diseases, requiring medical attention. Exemplary skin diseases in this regard are psoriasis, pemphigus, dermatitis, vitiligo, rosacea, mucous ulcers and mucous abnormalities. The “clinically healthy subjects” may be of all races and ethnicities and of all age, e.g. young, middle-age or elderly subjects, in each case male or female. Pregnant females are excluded from clinical studies with “clinically healthy subjects”; use of contraceptives is not limited, however. In clinical studies, the clinically healthy subjects are instructed not to use any topical preparations on the test areas starting from seven days prior to testing and until the end of the test. Furthermore, for cleansing only water and/or mild syndet (Eubos®, Dr. Hobein, D-53340 Meckenheim-Merl, Germany) is allowed for the whole study.

As used herein, the term “application to skin” intends to describe that the topical composition is applied to the skin, i.e. used on the skin, for cosmetic reasons. On the other hand, the term “administration to skin” intends to describe that the topical composition is pharmaceutically used on the skin for therapeutic reasons. Topical application or administration may mean that the medication is applied to body surfaces such as the skin or mucous membranes such as the vagina, anus, throat, nasal and oral cavity, nail, hair, eyes and ears. In particular, topical application or administration means directly laying on or spreading on outer skin, e.g. by use of hands or an applicator such as a wipe, puff, roller, or spray.

The influence of improving or maintaining the collagen structure of the skin on the skin properties is to be understood as follows: Collagen in skin is known to be responsible for main characteristics of skin, such as for example strength, elasticity, firmness, occurrence of wrinkles, smoothness, resilience tone, hydration, etc. Collagen undergoes the continuous physiological process of degradation that is simultaneously followed by its regeneration. This process is carried out practically every day since birth. However, the collagen matrix is partially degraded over time at diverse speeds in different individuals.

The collagen matrix of skin can be determined by methods described in the art, e.g. as described by M. Tanaka et al. (“Effects of Collagen Peptide Ingestion on UV-B-Induced Skin Damage”, Biosci. Biotechnol. Biochem., 73 (4), 930-932, 2009).

In particular, the collagen matrix can be determined by measurement of the skin thickness, preferably the skin thickness of the epidermis. On the other hand, if the skin thickness increases, the collagen structure of the skin is improved.

It is generally known that collagen structure determines the pivotal characteristics of skin. If collagen structure is impaired it results in impairment of the characteristics of skin and the opposite, if the collagen structure is improved, the characteristics of skin would be improved as well. If the collagen structure is improved, skin characteristics such as the biomechanical properties of the skin (skin fatigue, elasticity, firmness), skin roughness, skin hydration, wrinkle depth, area/volume and resistance to photooxidative stress are improved.

As used herein “maintaining or improving skin properties and/or skin characteristics” includes “maintaining or improving the biomechanical properties of skin”, “decreasing wrinkle depth, wrinkle area and/or wrinkle volume”, “protecting skin from oxidative stress”, “preventing or reducing skin roughness”, “maintaining or improving skin hydration”, and “maintaining or improving skin thickness”.

As used herein, “maintaining or improving the biomechanical properties of skin” includes inter alia positively influencing or regulating properties such as skin firmness, skin elasticity, and skin fatigue resistance. In particular, this term means enhancing the firmness or elasticity, preventing the loss of firmness and elasticity, preventing or treating sagging, lax and loose skin.

As used herein, “decreasing wrinkle depth, wrinkle area and/or wrinkle volume” means preventing, retarding, arresting, or reversing the process of wrinkle or fine line formation in skin, including, but not limited to, reducing the visibility or appearance of wrinkles.

As used herein, “protecting skin from oxidative stress” preferably means reduction or prevention of damage from external aggressions to skin by physical contact, chemical contact or temperature. External aggressions include use of chemicals such as cleansers and make up, physical contact by shaving, cutting, chemical contact with e.g. ozone, exhaust, chlorines, cigarette smoke, and damages caused by UV light. The term also includes increasing the elimination of free radicals developed under external aggressions.

As used herein, “preventing or reducing skin roughness” and “maintaining or improving skin hydration” result in regulating the texture of the skin, i.e. smoothing of the skin surface, removing bumps or crevasses, including, but not limited to, smoothing or evening the appearance of skin. Skin roughness and skin hydration are associated with transepidermal water loss. “Transepidermal water loss” is defined as the quantity of water that passes through the epidermal layer to the surrounding atmosphere via diffusion and evaporation processes.

As used herein, “maintaining or improving skin thickness” refers to maintaining or improving the epidermal skin thickness. Typically, if the epidermal skin thickness is increased, the collagen structure of the skin is improved at the same time.

As used herein “symptoms of skin diseases and/or disorders” include symptoms of skin irritation and/or neurodermitis and also cover “skin sensations selected from the group consisting of stinging, burning, tingling, itching, tickling, skin tightness, or any combinations thereof” and “skin redness”.

As used herein, “skin irritation” may be understood as meaning any change of the skin, which induces sensorial malaise in humans, preferably due to inflammation of the skin. Many intrinsic (e.g. genetic predisposition) and extrinsic (e.g. damage to the skin barrier, action of UV light, irritating and allergy-inducing substances) factors can lead to skin irritation. “Skin irritation” is typically correlated with “skin sensations selected from the group consisting of stinging, burning, tingling, itching, tickling, skin tightness, or any combinations thereof” and “skin redness”. A successful treatment of skin irritation is e.g. indicated by a reduction of the transepidermal water loss.

As used herein, “neurodermitis” also known as “atopic dermatitis” is an inflammatory, chronically relapsing, non-contagious and pruritic (itchy) skin disorder. The skin of a patient with atopic dermatitis reacts abnormally and easily to irritants, food, and environmental allergens and becomes red, flaky and very itchy. It also becomes vulnerable to surface infections caused by bacteria. The skin on the flexural surfaces of the joints (for example inner sides of elbows and knees) are the most commonly affected regions in people. A successful treatment of neurodermitis is e.g. indicated by a reduction of the transepidermal water loss.

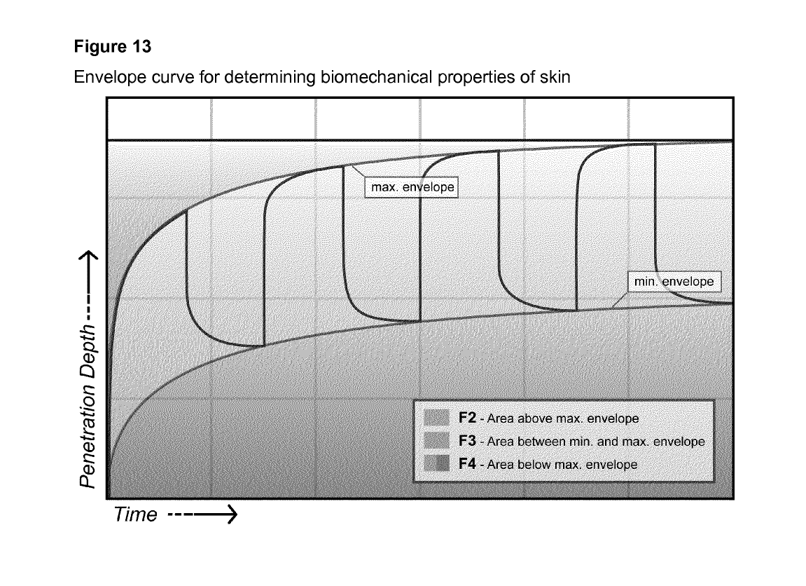

The biomechanical properties of skin can be determined by means of a cutometer. A cutometer is a commercially available non-invasive suction skin elasticity meter (see H. P. Dobrev, Folia Med (Plovdiv), 2002, 44(3):5-10). Preferably, a Cutometer MPA 580 (Courage & Khazaka GmbH, Cologne; S/N 41032492 tube: S/N 04317556) is used. The measurement is based on the vacuum-suction principle. By applying a constant negative pressure for a given period of time, skin is drawn into a hollow tube with an orifice of 2 mm in diameter. Then, at normal air pressure, skin is allowed to retract. The penetration depth of the skin into the tube is recorded optically without friction and without mechanical influence. This measurement is repeated several times to capture the properties of the skin under repeated external stress. When displaying the penetration depth (y) versus the time (t) in a diagram, several extensions will be recorded. Typically, the penetration depth increases under repeated external stress. Therefore, areas rather than individual measurement points are evaluated. Typically, an envelope curve of the minimum extensions and an envelope curve of the maximum extensions is approximated providing three areas, which are illustrated in FIG. 13.
Area F2: Area above the maximum envelope (limited by the y-value for the final extension)
Area F3: Area between the minimum and maximum envelope
Area F4: Area below the maximum envelope (limited by y=0)

The envelope curves of the minimum and maximum extensions can be approximated according to the following equation:

y=(ln x+b)/a
wherein:
   x=number of repetitions
   b=maximum amplitude
   a=minimum amplitude

Skin firmness can be assessed by the area F4. A decrease of F4 corresponds to an increase in skin firmness.

Skin elasticity is assessed by the ratio of the areas F3 and F4. The closer the value for F3/F4 is to 1, the more elastic is the skin.

Skin fatigue resistance is assessed by the area F2. A decrease of F2 corresponds to an increase in fatigue resistance.

The calculation can be performed by WinCT (Courage & Khazaki GmbH, Cologne—Germany)

The wrinkling of skin, preferably the wrinkling in Crow's feet, can be determined by measuring the wrinkle size, and in particular the wrinkle depth. The measurement of the wrinkle size by the parameters “mean wrinkle depth”, “mean wrinkle area”, “mean wrinkle volume”, and “mean wrinkle tissue reservoir volume” (WTRV) is e.g. described by J. Hatzis (J. Hatzis, Micron, 2004, 35(3): 201-19). The measurement is typically performed using PRIMOS.

PRIMOS (Phase shifting rapid in vivo measurement of skin) is a non-contact measurement device that allows for real time three-dimensional in vivo measurement of the micro topography of human skin based on the technology of active triangulation. The measurement head consists of a digital micromirror device as projection unit and a CCD-camera as recording unit, mounted onto an adjustable rack. For active image triangulation, an intensity encoded point M is projected onto the surface under investigation. Its image on the surface is recorded by the CCD-camera from a specific angle. The point M is a function of the parameters like intensity, triangulation angle between projection system and camera and some other inner respectively outer coordinates of the camera and projection plane. The height information of the structural surface is coded in the distorted intensity pattern, which is recorded. The resolution and accuracy depends on the optical and topographical characteristics of the measured surface and on the noise characteristics of the measurement system. For accurate in vivo measurements of human skin, depending on the measurement part of the human body, different parameters of effective wavelength and amplification factor can be used. To regard the differences of human skin and avoid undesired distortions by movements, the fast phase-shift technique with settings to accurately detect structures (phase width: 16, 64 & 128 pixels) can be used for the measurement. For each measurement, a minimum of 3 recordings are made and the clearest image without movement distortions or artefacts is selected for further processing. On follow-up visits, the original captured data is overlayed onto the skin of the volunteer to help in the relocation process of the test area. At the end of the study, distortions due to body hairs are digitally removed and the macro structure (calculated by polynominal approximation), i.e. the curvature of the entire test area, subtracted to allow a proper analysis of the micro structure, i.e. wrinkles and surface roughness. For the assessment by the “Wrinkle analysis module” of the PRIMOS software, the 3D-data from the follow-up visits are additionally elastically matched (alignment by shifting, rotation, tilting) and the maximum common area in the datasets from each subject is determined. As a result, the area covered for the analysis differs between subjects and hence absolute wrinkle volume and area measurements should not be compared between subjects.

For the purpose of the present invention, the wrinkle depth is preferably determined by means of PRIMOS® 5.7 high resolution (GFMeβtechnik GmbH, Teltow, Germany). In particular, the Crow's feet area is recorded as a 3D topography using the PRIMOS system (PRIMOS compact high-res. S/N 108-00041, software version 5.7.).

Wrinkle depth (μm) is assessed by means of the parameter Rₘₐₓ that is defined as the maximum vertical distance from the highest peak to the lowest valley of five segments of equal length.

Furthermore, the mean wrinkle depth (μm), the total wrinkle area (mm²) and the total wrinkle volume (mm³) can be determined. The mean wrinkle depth is calculated by the wrinkle analysis module. The total wrinkle area corresponds to the total measurement area covered by wrinkles and detected by the wrinkle analysis module. The total volume of wrinkles detected by the wrinkle analysis module can be calculated from the mean depth and the covered area.

The protection of skin from UV radiation, which typically causes photooxidative stress, can be assessed applying a β-carotene solution (5 mg/100 ml) in n-hexane (80 μl) to skin and irradiating the test sides with UVA radiation (UVA: 10 Joule/cm²). β-carotene is a natural substance having an orange-yellow color. Under the influence of UV light, it discolors as a result of a chemical process involving reactive oxygen species like singlet oxygen, the superoxide radical, hydrogen peroxide and the hydroxyl radical. If a topical composition or product applied to the skin has antioxidant properties, the color change of β-carotene is attenuated or inhibited. The color change can be determined by means of chromametry, preferably by using a

Chromameter CR 300 or CR 400 (S/N C8202118) in the L*a*b* colorimetry system (also designated as CIELAB system). CIE L*a*b* (CIELAB) is the most complete color space specified by the International Commission on Illumination (French Commission internationale de l'eclairage, hence its CIE initialism). It describes all the colors visible to the human eye and was created to serve as a device independent model to be used as a reference. The three coordinates of CIELAB represent the lightness of the color (L*=0 yields black and L*=100 indicates diffuse white; specular white may be higher), its position between red/magenta and green (a*, negative values indicate green while positive values indicate magenta) and its position between yellow and blue (b*, negative values indicate blue and positive values indicate yellow). The asterisk (*) after L, a and b are part of the full name, since they represent L*, a* and b*, to distinguish them from Hunter's L, a, and b.

During measurement, the skin surface is illuminated with a Xenon flashlight covering the entire visible spectrum, and remitted light is registered and analysed by a photoreceiver. The color of the reflected light is analysed by 3 high-sensitivity silicone photocells. Before each measuring series, the instrument is calibrated against a standard white.

The measurements are preferably performed according to the guidelines of the European Society of Contact Dermatitis (Fullerton et al., Guidelines for measurements of skin color and erythema, Contact Dermatitis 1996: 35, 1-10).

The results can be expressed as the ratio of inhibition of color change under UVA radiation, where a sample is applied (Sample), relative to the untreated area (Control), i.e. by the color index:

Color index=100−[(Sample_{colorized}−Sample_{irradiated})/(Control_{colorized}−Control_{irradiated})]×100

The color index in % directly corresponds to the efficacy against free radicals. Preferably, only the changes in the b* values, i.e. changes in the yellow color of the skin, are evaluated and expressed in the color index.

Skin roughness can be determined by Phase-shifting rapid in vivo measurement of skin (PRIMOS). PRIMOS is a non-contact measurement device, which allows for real time three dimensional in vivo measurement of the micro topography of human skin based on the technology of active image triangulation, as described above. To regard the differences of human skin and avoid undesired distortions by movements, the phase-shift technology was used for measurement. Distortions in the topography due to hairs can be digitally removed by the PRIMOS software. Skin roughness is assessed by means of the parameter R_{z} (mean depth of roughness). To mitigate potential directional effects, the evaluation was conducted using the arithmetic average of R_{z} from 32 radial cuts. The mean depth of roughness is defined as:

$${R}_{Z} = \frac{1}{n} {\sum }_{i = 1}^{n} {R}_{Zi}$$

where n is the number of equal segments into which the scan length 1 has been divided into and R_{z}, is the maximum peak to valley depth within each of the segments. In accordance with the German Standard Din 4768/1, R_{z} is calculated using 5 segments of equal length. A decrease of R_{z} corresponds to an increase in skin smoothness, i.e. a reduction of skin roughness.

Skin thickness, i.e. the thickness of epidermis plus dermis, can be assessed by echographic evaluation of the skin, preferably using COLLAGENOSON® “ICU” (Minhorst GmbH & Co., Meudt, Germany; S/N 7097). Said ultrasound device may be equipped with a 22 MHz ultrasonic transducer, which enables a high definition examination of the skin up to a depth of approximately 6 mm. Distilled water is usually employed as coupling medium between the probe and the skin surface. During each measurement of 1 second, where a probe is manually moved at a constant speed across the skin surface, 208 A-images (one-dimensional) are taken and merged to a B-image (two-dimensional) depicting a cross-section of the skin. A high gain curve is selected to compensate the relative low echogenicity of the facial skin. Skin thickness (epidermis+dermis) is measured as the distance between the first flank in the averaged A-image and the flank between the dermis and hypodermis. Usually, three measurements are performed on each test area and the mean is used to define skin thickness.

Transepidermal Water Loss (TEWL) measurements are preferably performed with a Tewameter™ 210 (Courage & Khazaka, Cologne, Germany). The tewameter is a device for measurement of water evaporation on skin surfaces based on the diffusion principle discovered by A. Fick in 1885. The TEWL is expressed in g/m² h.

The measurements of TEWL are preferably performed according to the guidelines of standardisation group of the European Contact Dermatitis Society (Pinnagoda et al., Contact Dermatitis, 1990: 22, 164-178).

Skin redness is determined by means of chromametry, preferably by using a Chromameter CR 400 (S/N C8202118) in the L*a*b* colorimetry system (also designated as CIELAB system) as defined above.

The measurements of skin redness are preferably performed according to the guidelines of the European Society of Contact Dermatitis (Fullerton et al., Guidelines for measurements of skin color and erythema, Contact Dermatitis 1996: 35, 1-10).

For assessment of the composition's or product's efficacy against skin redness and TEWL, the changes to the respective baseline values (e.g. 6 hours after SDS treatment to start, and 1, 2, 4, 7 days after SDS treatment has stopped) in the treated area compared to the untreated area are determined.

### DETAILED DESCRIPTION OF INVENTION

One embodiment of the present invention is directed to a topical composition comprising at least one angiotensin II receptor antagonist and at least one antioxidant.

In a preferred embodiment, the present invention is directed to a topical composition comprising at least one angiotensin II receptor antagonist and at least one antioxidant, wherein skin properties and/or characteristics are improved after application of the topical composition to skin for at least 14 days compared to the skin properties without application of the topical composition. Thus, the topical compositions according to the present invention are suitable for achieving a short-term improvement of skin properties and/or characteristics.

In another preferred embodiment, the present invention is directed to a topical composition comprising at least one angiotensin II receptor antagonist and at least one antioxidant, wherein symptoms of skin diseases and/or disorders are reduced after administration of the topical composition to skin for at least 14 days compared to the symptoms of skin diseases and/or disorders without administration of the topical composition. Thus, the topical compositions according to the present invention are suitable for achieving a short-term reduction of symptoms of skin diseases and/or disorders, such as symptoms of skin irritation and neurodermitis.

In one embodiment, the topical composition is for application or administration to skin, preferably human skin. Preferably, the topical composition is for application or administration to facial skin. Alternatively, the topical composition may be used for application or administration as an eye creme.

In another embodiment, the topical composition is for application or administration to apparently healthy skin, preferably apparently healthy human skin. Preferably, the topical composition is for application or administration to apparently healthy facial skin.

The angiotensin II receptor antagonist of the topical composition according to the present invention may be selected from the group consisting of azilsartan, losartan, eprosartan, irbesartan, olmesartan, candesartan, valsartan and telmisartan and any cosmetically or pharmaceutically acceptable salts, any cosmetically or pharmaceutically acceptable esters thereof and any combinations of the foregoing. Preferably, the angiotensin II receptor antagonist is selected from the group consisting of azilsartan, losartan, telmisartan, olmesartan, candesartan and valsartan and any cosmetically or pharmaceutically acceptable salts or any cosmetically or pharmaceutically acceptable esters thereof and any combinations of the foregoing. More preferably, the angiotensin II receptor antagonist is selected from the group consisting of losartan, telmisartan, azilsartan, candesartan and valsartan and any cosmetically or pharmaceutically acceptable salts or any cosmetically or pharmaceutically acceptable esters thereof and any combinations of the foregoing. Most preferably, the angiotensin II receptor antagonist is selected from the group consisting of losartan, valsartan and any cosmetically or pharmaceutically acceptable salts or any cosmetically or pharmaceutically acceptable esters thereof and any combinations thereof.

In one particularly preferred embodiment, the angiotensin II receptor antagonists of the topical composition according to the present invention is valsartan or any cosmetically or pharmaceutically acceptable salt thereof, preferably valsartan. In another particularly preferred embodiment, the angiotensin II receptor antagonist of the topical composition according to the present invention is losartan or any cosmetically or pharmaceutically acceptable salt thereof, preferably losartan potassium.

In one embodiment, the angiotensin II receptor antagonist is present in the topical composition according to the invention in an amount, which does not result in substantially lowering the systolic blood pressure or diastolic blood pressure upon topical application or administration to skin.

Preferably, the dose of the angiotensin II receptor antagonist in the topical composition according to the present invention does not produce any systemic effect—lowering blood pressure, therefore the beneficial effects at this dose are attributed solely/purely to the pleotropic effects of angiotensin II receptor antagonist. It is known from the state of the art that angiotensin II receptor antagonists when applied to the skin very poorly penetrate into deeper layers of skin and with difficulties reach arteries and capillaries and consequently systemic blood. It was ascertained that only a very small, insignificant amount of topically applied angiotensin II receptor antagonist might finally reach the systemic blood (Rizwan M. et al., Drug Dev. Ind. Pharm. 2008; 34: 618-26).

In a preferred embodiment, the angiotensin II receptor antagonist is present in an amount, which does not result in lowering the systolic blood pressure by more than about 15%, preferably not more than about 5%, more preferably not more than about 2%, most preferably not more than about 1% upon topical application or administration to skin. Preferably, the angiotensin II receptor antagonist is present in an amount, which does not result in lowering the systolic blood pressure by not more than about 15%, preferably not more than about 5%, more preferably not more than about 2%, most preferably not more than about 1% upon topical application or administration to skin, e.g. when the topical composition is applied or administered twice daily for 14, 21, 28, or 42 days preferably in an amount of about 2 mg/cm².

In another preferred embodiment, the angiotensin II receptor antagonist is present in an amount, which does not result in lowering the diastolic blood pressure by more than about 15%, preferably not more than about 5%, more preferably not more than about 2%, most preferably not more than about 1% upon topical application or administration to skin. Preferably, the angiotensin II receptor antagonist is present in an amount, which does not result in lowering the diastolic blood pressure by more than about 15%, preferably not more than about 5%, more preferably not more than about 2%, most preferably not more than about 1% upon topical application or administration to skin, e.g. when the topical composition is applied or administered twice daily for 14, 21, 28, or 42 days preferably in an amount of about 2 mg/cm².

In one embodiment, the angiotensin II receptor antagonist is present in the topical composition according to the invention in an amount of from about 0.1 to about 10 wt.-%, preferably from about 0.5 to about 5 wt.-%, more preferably from about 1 to about 3 wt.-%, most preferably in an amount of about 1.7 to about 2.3 wt.-% based on the total weight of the topical composition.

In another embodiment, the angiotensin II receptor antagonist is present in an amount of between 1 and 10 wt.-%, preferably between 1 and 5 wt.-% and more preferably between 1 and 3 wt.-% relative to the total weight of the topical composition.

In a preferred embodiment, the angiotensin II receptor antagonist in the topical composition according to the invention is valsartan or any cosmetically or pharmaceutically acceptable salt thereof, which is present in an amount of from about 0.1 to about 10 wt.-%, preferably from about 0.5 to about 5 wt.-%, more preferably from about 1 to about 3 wt.-%, most preferably in an amount of about 1.7 to about 2.3 wt.-% based on the total weight of the topical composition.

In another preferred embodiment, the angiotensin II receptor antagonist in the topical composition according to the present invention is losartan or any cosmetically or pharmaceutically acceptable salt thereof, which is present in an amount of from about 0.1 to about 10 wt.-%, preferably from about 0.5 to about 5 wt.-%, more preferably from about 1 to about 3 wt.-%, most preferably in an amount of about 1.7 to about 2.3 wt.-% based on the total weight of the topical composition.

The antioxidant of the topical composition according to the present invention may be selected from the group consisting of beta-carotene, ascorbic acid, retinol, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole and any cosmetically or pharmaceutically acceptable salts thereof and/or any analogues thereof and any combinations of the foregoing. Preferably, the antioxidant is selected from the group consisting of beta-carotene, ascorbic acid, tocopherol, tocopherol acetate, sodium ascorbyl phosphate, retinol, retinyl palmitate, coenzyme Q10 and resveratrol and any cosmetically or pharmaceutically acceptable salts thereof and/or any analogue thereof and any combinations of the foregoing. More preferably, the antioxidant is selected from the group consisting of tocopherol, tocopherol acetate, ascorbic acid, sodium ascorbyl phosphate, retinol, retinyl palmitate, coenzyme Q10, resveratrol and any combinations of the foregoing.

According to one aspect of the present invention the antioxidant is present in the pharmaceutical topical composition in the efficient amount to inhibit oxidation, as for example from 2 to 20 wt.-%, preferably from 3 to 15 wt.-% and more preferably from 4 to 10 wt.-% relative to the total weight of the topical composition.

In one embodiment, the antioxidant is present in the topical composition according to the present invention an amount of from about 2 to about 20 wt.-%, preferably from about 3 to about 18 wt.-% and more preferably from about 8 to about 15 wt.-% based on the total weight of the topical composition.

In a preferred embodiment, the antioxidant in the topical composition according to the present invention comprises any one of vitamin C, vitamin A, vitamin E, any salts thereof, any analogues thereof, or any combinations of the foregoing. Preferably, the antioxidant is selected from the group consisting of vitamin C, vitamin A, vitamin E, any cosmetically or pharmaceutically acceptable salts and analogues thereof, and any combinations of the foregoing. Preferably, the antioxidant is a combination of vitamin C, vitamin A, and vitamin E.

In a preferred embodiment, if the antioxidant is a combination of vitamin C, vitamin A and vitamin E,
a) vitamin C may be present in an amount of from about 1 to about 10 wt.-%, preferably from about 2 to about 8 wt.-%, more preferably from about 4 to about 6 wt.-%, most preferably about 5 wt.-% based on the total weight of the topical composition,
b) vitamin A may be present in an amount of from about 1 to about 10 wt.-%, preferably from about 2 to about 8 wt.-%, more preferably from about 4 to about 6 wt.-%, most preferably about 5 wt.-% based on the total weight of the topical composition, and
c) vitamin E may be present in an amount of from about 1 to about 10 wt.-%, preferably from about 2 to about 8 wt.-%, more preferably from about 4 to about 6 wt.-%, most preferably about 5 wt.-% based on the total weight of the topical composition.

In another embodiment, the antioxidant is present in the topical composition according to the present invention in an amount of from about 0.01 to about 30 wt.-%, preferably from about 0.05 to about 20 wt.-%, more preferably from about 0.1 to about 14 wt.-%, most preferably from about 0.3 to about 12 wt.-% based on the total weight of the topical composition.

In another preferred embodiment, the antioxidant of the topical composition according to the present invention is a combination of sodium ascorbyl phosphate (sodium vitamin C phosphate), retinyl palmitate (vitamin A palmitate), tocopherol (vitamin E) and tocopherol acetate (vitamin E acetate).

In a preferred embodiment, if the antioxidant is a combination of sodium ascorbyl phosphate (sodium vitamin C phosphate), retinyl palmitate (vitamin A palmitate), tocopherol (vitamin E) and tocopherol acetate (vitamin E acetate),
a) sodium ascorbyl phosphate (sodium vitamin C phosphate) may be present in an amount of from about 0.2 to about 8 wt.-%, preferably from about 0.4 to about 6 wt.-%, more preferably from about 0.6 to about 4 wt.-%, most preferably from about 0.8 to about 2 wt.-% based on the total weight of the topical composition,
b) retinyl palmitate (vitamin A palmitate) may be present in an amount of from about 0.01 to about 5 wt.-%, preferably from about 0.05 to about 3 wt.-%, more preferably from about 0.08 to about 1 wt.-%, most preferably from about 0.1 to about 0.5 wt.-% based on the total weight of the topical composition,
c) tocopherol (vitamin E) may be present in an amount of from about 0.01 to about 5 wt.-%, preferably from about 0.05 to about 3 wt.-%, more preferably from about 0.08 to about 1 wt.-%, most preferably from 0.1 to 0.5 wt.-% based on the total weight of the topical composition, and
d) tocopherol acetate (vitamin E acetate) may be present in an amount of from about 1 to about 10 wt.-%, preferably from about 2 to about 8 wt.-%, more preferably from about 3 to about 7 wt.-%, most preferably about 4 to about 6 wt.-% based on the total weight of the topical composition.

In yet another preferred embodiment, the antioxidant of the topical composition according to the present invention comprises coenzyme Q10, resveratrol, or a combination thereof.

According to one aspect of the invention, the topical composition according to the present invention preferably comprises at least one angiotensin II receptor antagonist selected from the group consisting of losartan, valsartan, azilsartan, candesartan, olmesartan, irbesartan and telmisartan and any cosmetically or pharmaceutically acceptable salts thereof and at least one antioxidant selected from the group consisting of vitamin A, vitamin C and vitamin E or any mixtures thereof.

According to one aspect of the invention, the topical composition according to the present invention comprises at least one angiotensin II receptor antagonist selected from the group consisting of losartan, telmisartan, azilsartan, candesartan and valsartan and any cosmetically or pharmaceutically acceptable salts or esters thereof in an amount between 1 and 10 wt.-%, preferably between 1 and 5 wt.-% and more preferably between 1 and 3 wt.-% relative to the total weight of the topical composition and at least one antioxidant selected from the group consisting of beta-carotene, ascorbic acid, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole and any cosmetically or pharmaceutically salts thereof and/or any analogues thereof or any mixtures thereof.

According to another aspect of the invention, the topical composition according to the present invention preferably comprises at least one angiotensin II receptor antagonist selected from the group consisting of losartan and valsartan and any cosmetically or pharmaceutically acceptable salts or esters thereof in an amount between 1 and 10 wt.-%, preferably between 1 and 5 wt.-% and more preferably between 1 and 3 wt.-% relative to the total weight of the topical composition and at least one antioxidant selected from the group consisting of tocopherol, tocopherol acetate, sodium ascorbyl phosphate, retinyl palmitate or any mixtures thereof.

In one embodiment, the angiotensin II receptor antagonist and the antioxidant are present in the topical composition according to the invention in an amount ratio of from about 1:2 to about 1:15, preferably from about 1:5 to about 1:10, more preferably from about 1:6 to about 1:9, most preferably about 1:7.5.

In another embodiment, the angiotensin II receptor antagonist and the antioxidant are present in the topical composition according to the present invention in an amount ratio of from about 2:1 to about 1:10, preferably from about 1:1 to 1:10, more preferably from about 1:1 to 1:6, most preferably from about 1:2 to about 1:5.

In one embodiment, the topical composition of the invention comprises at least one angiotensin II receptor antagonist and at least one antioxidant, wherein the angiotensin II receptor antagonist is losartan or any cosmetically or pharmaceutically acceptable salt thereof and the antioxidant comprises coenzyme Q10, resveratrol, or a combination thereof.

In another embodiment, the topical composition of the invention comprises at least one angiotensin II receptor antagonist and at least one antioxidant, wherein the angiotensin II receptorantagonist is valsartan or any cosmetically or pharmaceutically acceptable salt thereof and the antioxidant comprises coenzyme Q10, resveratrol, or a combination thereof.

In yet another embodiment, the topical composition of the invention comprises at least one angiotensin II receptor antagonist and at least one antioxidant, wherein the angiotensin II receptorantagonist is losartan or any cosmetically or pharmaceutically acceptable salt thereof and the antioxidant is selected from the group consisting of vitamin C, vitamin A, vitamin E, any cosmetically or pharmaceutically acceptable salts thereof, any cosmetically or pharmaceutically acceptable analogues thereof, and any combinations of the foregoing.

In yet another embodiment, the topical composition of the invention comprises at least one angiotensin II receptor antagonist and at least one antioxidant, wherein the angiotensin II receptorantagonist is valsartan or any cosmetically or pharmaceutically acceptable salt thereof and the antioxidant is selected from the group consisting of vitamin C, vitamin A, vitamin E, any cosmetically or pharmaceutically acceptable salts thereof, any cosmetically or pharmaceutically acceptable analogues thereof, and any combinations of the foregoing.

In one embodiment, the topical composition of the invention comprises at least one angiotensin II receptor antagonist and at least one antioxidant, wherein the angiotensin II receptor antagonist is losartan or any cosmetically or pharmaceutically acceptable salt thereof and the antioxidant is selected from the group consisting of vitamin C, vitamin A, vitamin E, any cosmetically or pharmaceutically acceptable salts and analogues thereof, and combinations of the foregoing. Preferably, the angiotensin II receptor antagonist is losartan or any cosmetically or pharmaceutically acceptable salt thereof and the antioxidant is a combination of vitamin C, vitamin A, and vitamin E. More preferably, the angiotensin II receptor antagonist is losartan potassium and is present in an amount of from about 1 to about 10 wt.-%, preferably from about 1 to about 5 wt.-%, more preferably from about 1 to about 3 wt.-%, most preferably in an amount of about 2 wt.-% relative to the total weight of the topical composition, and the antioxidant is a combination of vitamin C, vitamin A, vitamin E. Most preferably, the angiotensin II receptor antagonist is losartan potassium, and is present in an amount of from about 1 to about 10 wt.-%, preferably from about 1 to about 5 wt.-%, more preferably from about 1 to about 3 wt.-%, most preferably in an amount of about 2 wt.-% relative to the total weight of the topical composition, and the antioxidant is a combination of vitamin C, vitamin A, and vitamin E, wherein
a) vitamin C is present in an amount of from about 1 to about 10 wt.-%, preferably from about 2 to about 8 wt.-%, more preferably from about 4 to about 6 wt.-%, most preferably about 5 wt.-% based on the total weight of the topical composition, wherein
b) vitamin A is present in an amount of from about 1 to about 10 wt.-%, preferably from about 2 to about 8 wt.-%, more preferably from about 4 to about 6 wt.-%, most preferably about 5 wt.-% based on the total weight of the topical composition, and wherein
c) vitamin E is present in an amount of from about 1 to about 10 wt.-%, preferably from about 2 to about 8 wt.-%, more preferably from about 4 to about 6 wt.-%, most preferably about 5 wt.-% based on the total weight of the topical composition.

In another embodiment, the topical composition of the invention comprises at least one angiotensin II receptor antagonist and at least one antioxidant, wherein the angiotensin II receptor antagonist is valsartan or any cosmetically or pharmaceutically acceptable salt thereof and the antioxidant is selected from the group consisting of vitamin C, vitamin A, vitamin E, any cosmetically or pharmaceutically acceptable salts and analogues thereof, and combinations of the foregoing. Preferably, the angiotensin II receptor antagonist is valsartan or any cosmetically or pharmaceutically acceptable salt thereof and the antioxidant is selected from the group consisting of sodium ascorbyl phosphate (sodium vitamin C phosphate), retinyl palmitate (vitamin A palmitate), tocopherol (vitamin E) and tocopherol acetate (vitamin E acetate), and any combinations of the foregoing. More preferably, the angiotensin II receptor antagonist is valsartan and is present in an amount of from about 1 to about 10 wt.-%, preferably from about 1 to about 5 wt.-%, more preferably from about 1 to about 3 wt.-%, most preferably in an amount of about 2 wt.-% relative to the total weight of the topical composition, and the antioxidant is a combination of sodium ascorbyl phosphate (sodium vitamin C phosphate), retinyl palmitate (vitamin A palmitate), tocopherol (vitamin E) and tocopherol acetate (vitamin E acetate). Most preferably, the angiotensin II receptor antagonist is valsartan, and is present in an amount of from about 1 to about 10 wt.-%, preferably from about 1 to about 5 wt.-%, more preferably from about 1 to about 3 wt.-%, most preferably in an amount of about 2 wt.-% relative to the total weight of the topical composition, and the antioxidant is a combination of sodium ascorbyl phosphate (sodium vitamin C phosphate), retinyl palmitate (vitamin A palmitate), tocopherol (vitamin E) and tocopherol acetate (vitamin E acetate),

wherein
a) sodium ascorbyl phosphate (sodium vitamin C phosphate) is present in an amount of from about 0.2 to about 8 wt.-%, preferably from about 0.4 to about 6 wt.-%, more preferably from about 0.6 to about 4 wt.-%, most preferably from about 0.8 to about 2 wt.-% based on the total weight of the topical composition, wherein
b) retinyl palmitate (vitamin A palmitate) is present in an amount of from about 0.01 to about 5 wt.-%, preferably from about 0.05 to about 3 wt.-%, more preferably from about 0.08 to about 1 wt.-%, most preferably from about 0.1 to about 0.5 wt.-% based on the total weight of the topical composition, wherein
c) tocopherol (vitamin E) is present in an amount of from about 0.01 to about 5 wt.-%, preferably from about 0.05 to about 3 wt.-%, more preferably from about 0.08 to about 1 wt.-%, most preferably from about 0.1 to about 0.5 wt.-% based on the total weight of the topical composition,
   and wherein
d) tocopherol acetate (vitamin E acetate) is present in an amount of from about 1 to about 10 wt.-%, preferably from about 2 to about 8 wt.-%, more preferably from about 3 to about 7 wt.-%, most preferably from about 4 to about 6 wt.-% based on the total weight of the topical composition.

In one embodiment, the topical composition according to the present invention further comprises a triglyceride, preferably caprylic/capric triglyceride. Said compound is preferably used as a solvent, when preparing a mixture of vitamin A palmitate and vitamin E to be used as antioxidants in the topical composition of the invention.

The pharmaceutical topical composition according to the present invention may contain further pharmaceutically acceptable ingredients suitable for topical application that should be skin and mucosa tolerant, non-allergic, well penetrating, lipophilic, chemically stable and bioactive. According to the present invention there is provided a pharmaceutical topical composition that may further comprise one or more of penetration enhancers, thickeners, stiffening agents, solvents, emulsifiers, emollients, preservatives, buffers, vehicles, fragrances, coloring agents, chelating agents, humectants and mixtures thereof.

In one embodiment, the topical composition according to the present invention may further comprise at least one further ingredient selected from the group consisting of 2-hydroxy-4-methoxybenzophenone, 1-phenyl-3(4′-isopropylphenyl)propane-1,3-dione, macrogel stearate, cetylstearyl alcohol, 1,2-propandiole, and vaseline.

In another embodiment, the topical composition according to the present invention may further comprise at least one further ingredient selected from the group consisting of cetearyl alcohol, cetearyl glucoside, alcohol denat., polyacrylamide, C₁₃₋₁₄ isoparaffin, laureth-7, glycerin, dicaprylyl carbonate, dicaprylyl ether, propylheptyl caprylate, dimethicone, stearic acid, cetyl alcohol, stearyl alcohol, triethanolamine, phenoxyethanol, ethylhexylglycerin, disodium EDTA, perfume, and water.

In one embodiment, the topical composition according to the present invention may further comprise at least one cosmetic ingredient according to INCI system (International Nomenclature of Cosmetic Ingredients) selected from the listed consisting of, but not limited to, abrasives (e.g. to remove materials from various body surfaces or to aid mechanical tooth cleaning or to improve gloss), absorbents (e.g. to take up water- and/or oil-soluble dissolved or finely dispersed substances), additives (e.g. to impart or improve desirable properties or suppress (or minimise) undesirable properties), anticorrosives (e.g. to avoid corrosion of the packaging), anti-dandruff agents (e.g. to control dandruff of hair), anti-foaming agents (e.g. to suppress foam during manufacturing or to reduce the tendency of finished products to generate foam), antimicrobials (e.g. to help reduce the activities of microorganisms on the skin or body), antiperspirants (e.g. to reduce perspiration), antistatic agents (e.g. to reduce static electricity by neutralising electrical charge on a surface), binders (e.g. to provide cohesion), biological additives (e.g. to achieve specific formulation features), bleaching agents (e.g. to lighten the shade of hair or skin), botanicals (e.g. derived from plants), buffering agents (e.g. to adjust or stabilize the pH), chelating agents (e.g. to react and to form complexes with metal ions which could affect stability and/or appearance of cosmetics), colorants (e.g. to colour the cosmetic product and/or to impart colour to the skin and/or its appendages), denaturants (e.g. to render cosmetic products unpalatable), deodorants (e.g. to reduce or mask unpleasant body odours), depilating agents (e.g. to remove unwanted body hair), emollients (e.g. to soften and smoothen the skin), emulsifiers (e.g. surface-active agents that promote the formation of intimate mixtures of immiscible liquids), emulsion stabilizers (e.g. to help the process of emulsification and to improve formulation stability and shelf-life), film-formers (e.g. to produce, upon application, a continuous film on skin, hair or nails), hair dyes (e.g. to colour hair), humectants (e.g. to hold and retain moisture), opacifiers (e.g. to render cosmetic products more impervious to visible light and nearby radiation), oral care agents (e.g. for the care of the oral cavity), oxidizing agents (e.g. to change the chemical nature of another substance by adding oxygen), preservatives (e.g. to inhibit the development of microorganisms), propellants (e.g. gaseous substances added to cosmetic products under pressure in pressure-resistant containers for expelling the contents of the containers when the pressure is released), reducing agents (e.g. to change the chemical nature of another substance by adding hydrogen or removing oxygen), solvents (e.g. to dissolve components), surfactants (to lower the surface tension and to aid the even distribution of the cosmetic product), UV absorbers (e.g. to filter certain UV rays in order to protect the skin or the products from certain harmful effects of these rays), viscosity enhancers (e.g. to increase or decrease the viscosity of the cosmetic product) and any combinations thereof.

Preferably, the topical composition of the invention further comprises a cosmetic ingredient according to the INCI system selected from the group consisting of abrasives, absorbents, additives, anticorrosives, anti-dandruff agents, anti-foaming agents, antimicrobials, antiperspirants, antistatic agents, binders, biological additives, bleaching agents, botanicals, buffering agents, chelating agents, colorants, denaturants, deodorants, depilating agents, emollients, emulsifiers, emulsion stabilizers, film-formers, hair dyes, humectants, opacifiers, oral care agents, oxidizing agents, preservatives, propellants, reducing agents, solvents, surfactants, UV absorbers, viscosity enhancers and any combinations of the foregoing.

In another embodiment, the topical composition of the invention further comprises an ingredient selected from the group consisting of penetration enhancers, thickeners, stiffening agents, solvents, emulsifiers, emollients, preservatives, buffers, vehicles, fragrances, coloring agents, chelating agents, humectants and any combinations of the foregoing.

In yet another embodiment, the topical composition according to the present invention may further comprise at least one ingredient selected from the list consisting of, but not limited to, exfoliants, anti-acne agents, anti-aging agents, anti-dark circles agents, anti-wrinkle agents, conditioners, hair repair agents, nourishing agents, pearlizers, perfume ingredients, self-tanners, skin-lightening agents, sunscreens and similar and any combinations thereof.

Preferably, the topical composition of the invention further comprises an ingredient selected from the group consisting of exfoliants, anti-acne agents, anti-aging agents, anti-dark circles agents, anti-wrinkle agents, conditioners, hair repair agents, nourishing agents, pearlizers, perfume ingredients, self-tanners, skin-lightening agents, sunscreens and any combinations of the foregoing.

In yet another embodiment, the topical composition of the invention further comprises at least one excipient. In one embodiment the excipient is a pharmaceutically acceptable excipient. In another embodiment, the excipient is a cosmetically acceptable excipient.

In another embodiment, the topical composition according to the present invention does not comprise an UVA and/or UVB filter. Preferably, the topical composition does not comprise a UV filter. More preferably, the topical composition according to the present invention does not comprise a barrier compound. Most preferably, the composition according to the present invention does not comprise a barrier compound, which acts as a sunscreen.

In another embodiment, the topical composition according to the present invention does not comprise a barrier compound selected from 2-hydroxy-4-methoxybenzophenone and 1-phenyl-3-(4′-isopropylphenyl)propane-1,3-dione.

In another embodiment, the topical composition according to the present invention comprises valsartan or a cosmetically or pharmaceutically acceptable salt thereof as angiotensin II receptor antagonist, and does not comprise a barrier compound selected from 2-hydroxy-4-methoxybenzophenone and 1-phenyl-3-(4′-isopropylphenyl)propane-1,3-dione.

In one embodiment, the topical composition according to the present invention essentially consists of
a) at least one angiotensin II receptor antagonist, preferably losartan or a cosmetically or pharmaceutically acceptably salt thereof
b) at least one antioxidant, and
c) at least one further ingredient selected from the group consisting of 2-hydroxy-4-methoxybenzophenone, 1-phenyl-3(4′-isopropylphenyl)propane-1,3-dione, macrogel stearate, cetylstearyl alcohol, 1,2-propandiole, and vaseline.

In another embodiment, the topical composition according to the present invention essentially consists of
a) at least one angiotensin II receptor antagonist, preferably valsartan or a cosmetically or pharmaceutically acceptable salt thereof
b) at least one antioxidant, and
c) at least one further ingredient selected from the group consisting of cetearyl alcohol, cetearyl glucoside, alcohol denat., polyacrylamide, C₁₃₋₁₄ isoparaffin, laureth-7, glycerin, dicaprylyl carbonate, dicaprylyl ether, propylheptyl caprylate, dimethicone, stearic acid, cetyl alcohol, stearyl alcohol, triethanolamine, phenoxyethanol, ethylhexylglycerin, disodium EDTA, perfume, and water.

The topical composition according to the present invention is suitable for topical application or administration may take the form of, but are not limited to, liquids, semi-solids or solids. Liquids may be in the form of emulsions, suspensions, solutions, sprays or shampoos. Semi-solids may be in the form of collodions, foams, ointments, pastes, creams or gels. Solids may be of powders, aerosols, patches, gauzes, compress, tapes, sticks or tablets for topical application. Preferably, it may be in the form of cream, lotion or skin patch, more preferably in the form of cream.

Preferably, the topical composition according to the present invention may be in the form of
a) liquids, preferably selected from the group consisting of emulsions, suspensions, solutions, sprays or shampoos,
b) semi-solids, preferably selected from the group consisting of colloids, foams, ointments, pastes, creams or gels, or
c) solids, preferably selected from the group consisting of powders, aerosols, patches, gauzes, compress, tapes, sticks or tablets for topical application or administration.

Preferably, the topical composition is in the form of a cream, a lotion or a skin patch. More preferably, the topical composition is in the form of a cream, preferably a face cream. Alternatively, the topical composition is in the form of an eye cream.

Emulsions according to the present invention are two-phase systems in which one liquid is dispersed throughout another liquid in the form of small droplets. The system can be designated as an oil-in-water emulsion or water-in-oil emulsion. Emulsions are stabilized by emulsifying agents that prevent coalescence, the merging of small droplets into larger droplets and, ultimately, into a single separated phase. Emulsifying agents are concentrated in the interface between the droplet and external phase and by providing a physical barrier around the particle to coalescence. They also reduce the interfacial tension between the phases, thus increasing the ease of emulsification upon mixing. Natural, semisynthetic, and synthetic hydrophilic polymers may be used as emulsifying agent. The emulsions may require an antimicrobial agent because the aqueous phase is favorable to the growth of microorganisms.

Creams according to the present invention are semisolid dosage forms containing the active substance dissolved or dispersed in a suitable base such as for example olive oil, tons oil, rose oil and similar. The semisolids possess a relatively fluid consistency formulated as either water-in-oil or oil-in-water emulsions or aqueous microcrystalline dispersions.

Gels are semisolid systems consisting of suspensions made up of small inorganic particles or large organic molecules interpenetrated by a liquid. Where the gel mass consists of a network of small discrete particles, the gel is classified as a two-phase system. Single-phase gels consist of organic macromolecules uniformly distributed throughout a liquid in such a manner that no apparent boundaries exist between the dispersed macromolecules and the liquid. Single-phase gels may be made from synthetic macromolecules or from natural gums. Gels may be aqueous or they may contain alcohols and/or oils as the continuous phase.

The topical composition according to the present invention may be combined as a separate topical composition to at least one other pharmaceutical or cosmetic topical composition or it may be incorporated within another pharmaceutical or cosmetic topical composition.

The topical composition according to the present invention was found to be stable and homogeneous in the time period analyzed. There was no change in pH influenced by temperature or time or light. No microbiological contamination was detected. Organoleptic and sensorial tests showed a very good acceptance in terms of appearance, smell and color, uniformity and good absorption, making it the product of choice in the double-blind study. Accelerated stability studies showed preservation for a period of at least 2 years.

The topical composition according to the present invention may be prepared by conventional techniques well known to those skilled in the art.

The topical composition according to the present invention may be applied or administered on skin or mucosa up to 5 times daily, preferable 3 times daily, more preferably 2 times daily.

Preferably, the topical composition is applied or administered to skin at least once in a week, preferably at least once in two days, more preferably at least once daily, most preferably at least twice daily. Preferably, the topical composition is applied or administered to skin for at least 1 day, preferably at least 2 days, more preferably at least 7 days, or for at least 14 days, preferably for at least 28 days, or for at least 21 days, preferably at least 42 days.

In a preferred embodiment, the topical composition according to the present invention is applied or administered to skin in an amount of at least about 0.1 mg/cm², preferably at least about 0.5 mg/cm², more preferably at least about 1 mg/cm², most preferably at least about 2 mg/cm².

Preferably, the topical composition is applied or administered to skin at least twice daily for at least 1 day, preferably at least 7 days, more preferably in an amount of at least about 2 mg/cm², or at least twice daily for at least 14 days, preferably 28 days, more preferably in an amount of at least about 2 mg/cm², or at least twice daily for at least 21 days, preferably 42 days, more preferably in an amount of at least about 2 mg/cm².

The topical composition for topical application or administration according to the present invention can be used for cosmetic and/or for medical purposes. As regards the cosmetic purposes, in particular “day-to-day” changes of skin properties and/or characteristics are addressed.

The topical composition according to the present invention is suitable for subjects of whatever age: young, middle-age or elderly since the structure of skin can be impaired due to several reasons independently on ages, as for example exposure to sun, acnes, pollution, influence of hormones, ageing etc.

In particular, the topical composition according to the present invention is suitable for improving and/or maintaining collagen structure at any age independent of sex.

In one embodiment, the topical composition of the present invention for topical application or administration comprising at least one angiotensin II receptor antagonist and at least one antioxidant or any mixtures thereof is suitable for use in improving and/or maintaining collagen structure of skin, preferably apparently healthy skin. Preferably, the topical composition is suitable for use in improving and/or maintaining collagen structure of skin, preferably apparently healthy skin, wherein the topical composition is applied to body surfaces such as the skin or mucous membranes.

Another aspect of the present invention is the topical composition for topical application or administration comprising at least one angiotensin II receptor antagonist and at least one antioxidant or any mixtures thereof for use in improving and/or maintaining collagen structure of skin, preferably apparently healthy skin, wherein the amount of angiotensin II receptor antagonist is between 1 and 10 wt.-%, preferably between 1 and 5 wt.-%, more preferably between 1 and 3 wt.-%, and most preferably between 1.7 to 2.3 wt.-% relative to the total weight of the topical composition.

According to another aspect of the invention, the topical composition according to the present invention preferably comprises at least one angiotensin II receptor antagonist selected from the group consisting of losartan, valsartan, azilsartan, candesartan, olmesartan, irbesartan and telmisartan and any cosmetically or pharmaceutically acceptable salts thereof and at least one antioxidant selected from the group consisting of vitamin A, vitamin C and vitamin E or any mixtures thereof, and is for use in improving and/or maintaining collagen structure of skin, preferably apparently healthy skin.

According to another aspect of the invention, the topical composition according to the present invention preferably comprises at least one angiotensin II receptor antagonist selected from the group consisting of losartan, telmisartan, azilsartan, candesartan and valsartan and any cosmetically or pharmaceutically acceptable salts or esters thereof in an amount between 1 and 10 wt.-%, preferably between 1 and 5 wt.-% and more preferably between 1 and 3 wt.-% relative to the total weight of the topical composition and at least one antioxidant selected from the group consisting of beta-carotene, ascorbic acid, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole and any cosmetically or pharmaceutically acceptable salts thereof and/or any analogues thereof or any mixtures thereof, and is for use in improving and/or maintaining collagen structure of skin, preferably apparently healthy skin.

According to yet another aspect of the invention, the topical composition according to the present invention preferably comprises at least one angiotensin II receptor antagonist selected from the group consisting of losartan and valsartan and any cosmetically or pharmaceutically acceptable salts or esters thereof in an amount between 1 and 10 wt.-%, preferably between 1 and 5 wt.-%, more preferably between 1 and 3 wt.-%, most preferably between 1.7 and 2.3 wt.-% relative to the total weight of the topical composition and at least one antioxidant selected from the group consisting of tocopherol, tocopherol acetate, sodium ascorbyl phosphate, retinyl palmitate or any mixtures thereof, and is for use in improving and/or maintaining collagen structure of skin, preferably apparently healthy skin.

In one embodiment, the topical composition according to the present invention is suitable for improving skin properties and skin characteristics.

When describing the improvement of the skin properties and skin characteristics in the following, it is also referred to a “mean” improvement. As used herein the term “mean” describes the improvement of the skin properties and skin characteristics mediated over a group of at least 5, preferably at least 10, more preferably at least 15, most preferably about 20 tested subjects. As used herein, the “mean” value is the arithmetic mean value.

In one aspect, the skin firmness is increased after application of the topical composition of the present invention compared to the skin firmness without application of the topical composition.

In a preferred embodiment, the skin firmness is increased by at least 1%, preferably by at least 3%, more preferably by at least 5%, most preferably by at least 8% after application of the topical composition, preferably after application for at least 21 days, more preferably after application for at least 14 days, compared to the skin firmness without application of the topical composition.

In another preferred embodiment, the mean skin firmness is increased by at least 1%, preferably by at least 3%, more preferably by at least 5%, most preferably by at least 8% after application of the topical composition, preferably after application for at least 21 days, more preferably after application for at least 14 days, compared to the mean skin firmness without application of the topical composition.

In another preferred embodiment, the skin firmness is increased by at least 5%, preferably by at least 10%, more preferably by at least 12%, most preferably by at least 14% after application of the topical composition, preferably after application for at least 42 days, more preferably after application for at least 28 days, compared to the skin firmness without application of the topical composition.

In another preferred embodiment, the mean skin firmness is increased by at least 5%, preferably by at least 10%, more preferably by at least 12%, most preferably by at least 14% after application of the topical composition, preferably after application for at least 42 days, more preferably after application for at least 28 days, compared to the mean skin firmness without application of the topical composition.

In another aspect, the skin elasticity is increased after application of the topical composition of the present invention compared to the skin elasticity without application of the topical composition.

In a preferred embodiment, the skin elasticity is increased by at least 1%, preferably by at least 3%, more preferably by at least 5%, most preferably by at least 6% after application of the topical composition, preferably after application for at least 21 days, more preferably after application for at least 14 days, compared to the skin elasticity without application of the topical composition.

In another preferred embodiment, the mean skin elasticity is increased by at least 1%, preferably by at least 3%, more preferably by at least 5%, most preferably by at least 6% after application of the topical composition, preferably after application for at least 21 days, more preferably after application for at least 14 days, compared to the mean skin elasticity without application of the topical composition.

In another preferred embodiment, the skin elasticity is increased by at least 3%, preferably by at least 5%, more preferably by at least 7%, most preferably by at least 9% after application of the topical composition, preferably after application for at least 42 days, more preferably after application for at least 28 days, compared to the skin elasticity without application of the topical composition.

In another preferred embodiment, the mean skin elasticity is increased by at least 3%, preferably by at least 5%, more preferably by at least 7%, most preferably by at least 9% after application of the topical composition, preferably after application for at least 42 days, more preferably after application for at least 28 days, compared to the mean skin elasticity without application of the topical composition.

In yet another aspect, the skin fatigue resistance is increased after application of the topical composition of the present invention compared to the skin fatigue resistance without application of the topical composition.

In a preferred embodiment, the skin fatigue resistance is increased by at least 3%, preferably by at least 5%, more preferably by at least 7%, most preferably by at least 9% after application of the topical composition, preferably after application for at least 21 days, more preferably after application for at least 14 days, compared to the skin fatigue resistance without application of the topical composition.

In another preferred embodiment, the mean skin fatigue resistance is increased by at least 3%, preferably by at least 5%, more preferably by at least 7%, most preferably by at least 9% after application of the topical composition, preferably after application for at least 21 days, more preferably after application for at least 14 days, compared to the mean skin fatigue resistance without application of the topical composition.

In another preferred embodiment, the skin fatigue resistance is increased by at least 4%, preferably by at least 6%, more preferably by at least 8%, most preferably by at least 10% after application of the topical composition, preferably after application for at least 42 days, more preferably after application for at least 28 days, compared to the skin fatigue resistance without application of the topical composition.

In another preferred embodiment, the mean skin fatigue resistance is increased by at least 4%, preferably by at least 6%, more preferably by at least 8%, most preferably by at least 10% after application of the topical composition, preferably after application for at least 42 days, more preferably after application for at least 28 days, compared to the mean skin fatigue resistance without application of the topical composition.

In yet another aspect, the wrinkle depth, wrinkle area and/or wrinkle volume of wrinkles of skin, preferably of inner side of forearms or face, more preferably Crow's feet, are reduced after application of the topical composition compared to the wrinkle depth, wrinkle area and/or wrinkle volume of wrinkles of skin, preferably of inner side of forearms or face, more preferably Crow's feet, without application of the topical composition.

In a preferred embodiment, the wrinkle depth, preferably the wrinkle depth in Crow's feet, is reduced by at least 2%, preferably by at least 4%, more preferably by at least 6%, most preferably by at least 8% after application of the topical composition, preferably after application for at least 21 days, more preferably after application for at least 14 days, compared to the wrinkle depth without application of the topical composition.

In another preferred embodiment, the mean wrinkle depth, preferably the mean wrinkle depth in Crow's feet, is reduced by at least 2%, preferably by at least 4%, more preferably by at least 6%, most preferably by at least 8% after application of the topical composition, preferably after application for at least 21 days, more preferably after application for at least 14 days, compared to the mean wrinkle depth without application of the topical composition.

In another preferred embodiment, the wrinkle depth, preferably the wrinkle depth in Crow's feet, is reduced by at least 5%, preferably by at least 8%, more preferably by at least 12%, most preferably by at least 13% after application of the topical composition, preferably after application for at least 21 days, more preferably after application for at least 14 days, compared to the wrinkle depth without application of the topical composition.

In another preferred embodiment, the mean wrinkle depth, preferably the mean wrinkle depth in Crow's feet, is reduced by at least 5%, preferably by at least 8%, more preferably by at least 12%, most preferably by at least 15% after application of the topical composition, preferably after application for at least 42 days, more preferably after application for at least 28 days, compared to the mean wrinkle depth without application of the topical composition.

In another preferred embodiment, the wrinkle area, preferably the wrinkle area in Crow's feet, is reduced by at least 0.1%, preferably by at least 0.5%, more preferably by at least 1.5%, most preferably by at least 2.5% after application of the topical composition, preferably after application for at least 21 days, more preferably after application for at least 14 days, compared to the wrinkle area without application of the topical composition.

In another preferred embodiment, the mean wrinkle area, preferably the mean wrinkle area in Crow's feet, is reduced by at least 0.1%, preferably by at least 0.5%, more preferably by at least 1.5%, most preferably by at least 2.5% after application of the topical composition, preferably after application for at least 21 days, more preferably after application for at least 14 days, compared to the mean wrinkle area without application of the topical composition.

In another preferred embodiment, the wrinkle area, preferably the wrinkle area in Crow's feet, is reduced by at least 4%, preferably by at least 7%, more preferably by at least 10%, most preferably by at least 13% after application of the topical composition, preferably after application for at least 42 days, more preferably after application for at least 28 days, compared to the wrinkle area without application of the topical composition.

In another preferred embodiment, the mean wrinkle area, preferably the mean wrinkle area in Crow's feet, is reduced by at least 4%, preferably by at least 7%, more preferably by at least 10%, most preferably by at least 13% after application of the topical composition, preferably after application for at least 42 days, more preferably after application for at least 28 days, compared to the mean wrinkle area without application of the topical composition.

In another preferred embodiment, the wrinkle volume, preferably the wrinkle volume in Crow's feet, is reduced by at least 3%, preferably by at least 6%, more preferably by at least 9%, most preferably by at least 11% after application of the topical composition, preferably after application for at least 21 days, more preferably after application for at least 14 days, compared to the wrinkle volume without application of the topical composition.

In another preferred embodiment, the mean wrinkle volume, preferably the mean wrinkle volume in Crow's feet, is reduced by at least 3%, preferably by at least 6%, more preferably by at least 9%, most preferably by at least 11% after application of the topical composition, preferably after application for at least 21 days, more preferably after application for at least 14 days, compared to the mean wrinkle volume without application of the topical composition.

In another preferred embodiment, the wrinkle volume, preferably the wrinkle volume in Crow's feet, is reduced by at least 8%, preferably by at least 14%, more preferably by at least 20%, most preferably by at least 25% after application of the topical composition, preferably after application for at least 42 days, more preferably after application for at least 28 days, compared to the wrinkle volume without application of the topical composition.

In another preferred embodiment, the mean wrinkle volume, preferably the mean wrinkle volume in Crow's feet, is reduced by at least 8%, preferably by at least 14%, more preferably by at least 20%, most preferably by at least 25% after application of the topical composition, preferably after application for at least 42 days, more preferably after application for at least 28 days, compared to the mean wrinkle volume without application of the topical composition.

In yet another aspect, the oxidative stress is reduced after application of the topical composition of the present invention compared to the oxidative stress without application of the topical composition.

In a preferred embodiment, oxidative stress is reduced by at least 10%, preferably by at least 20%, more preferably by at least 27%, most preferably by at least 28% after application of the topical composition, preferably after a single application of the topical composition, compared to the oxidative stress without application of the topical composition.

In another preferred embodiment, the mean oxidative stress is reduced by at least 10%, preferably by at least 20%, more preferably by at least 27%, most preferably by at least 28% after application of the topical composition, preferably after a single application of the topical composition, compared to the mean oxidative stress without application of the topical composition.

In yet another aspect, the skin roughness is reduced after application of the topical composition of the present invention compared to the skin roughness without application of the topical composition.

In a preferred embodiment, the skin roughness is reduced by at least 5%, preferably by at least 8%, more preferably by at least 10%, most preferably by at least 12%, preferably after application for at least 21 days, preferably for at least 14 days, compared to the skin roughness without application of the topical composition.

In another preferred embodiment, the mean skin roughness is reduced by at least 5%, preferably by at least 8%, more preferably by at least 10%, most preferably by at least 12%, preferably after application for at least 21 days, preferably for at least 14 days, compared to the mean skin roughness without application of the topical composition.

In a preferred embodiment, the skin roughness is reduced by at least 8%, preferably by at least 10%, more preferably by at least 12%, most preferably by at least 13%, preferably after application for at least 42 days, preferably for at least 28 days, compared to the skin roughness without application of the topical composition.

In a preferred embodiment, the mean skin roughness is reduced by at least 8%, preferably by at least 10%, more preferably by at least 12%, most preferably by at least 13%, preferably after application for at least 42 days, preferably for at least 28 days, compared to the mean skin roughness without application of the topical composition.

In yet another aspect, transepidermal water loss is reduced and/or prevented after application of the topical composition of the present invention compared to transepidermal water loss without application of the topical composition.

In a preferred embodiment, transepidermal water loss is reduced, indicated by a reduction of the transepidermal water loss, which is at least 2.5%, preferably at least 10%, more preferably at least 15%, most preferably at least 20% greater after application of the topical composition for at least 1 day, preferably for at least 2 days, compared to the reduction of the transepidermal water loss without application of the topical composition.

In another preferred embodiment, transepidermal water loss is prevented, indicated by an smaller increase of the transepidermal water loss, which is at least 4% less, preferably 12% less, more preferably 19% less, most preferably 23% less after application of the topical composition together with sodium dodecyl sulphate, for at least 1 day, preferably at least 2 days, compared to the increase of the transepidermal water loss after application of sodium dodecyl sulphate without application of the topical composition.

In yet another aspect, skin thickness is increased after application of the topical composition of the present invention compared to the skin thickness without application of the topical composition.

In a preferred embodiment, skin thickness is increased by at least 0.005 mm, preferably by at least 0.01 mm, more preferably by at least 0.03 mm, most preferably by at least 0.04 mm after application of the topical composition, preferably after application for at least 21 days, more preferably after application for at least 14 days, compared to skin thickness without application of the topical composition.

In another preferred embodiment, skin thickness is increased by at least 0.01 mm, preferably by at least 0.03 mm, more preferably by at least 0.06 mm, most preferably by at least 0.08 mm after application of the topical composition, preferably after application for at least 42 days, more preferably after application for at least 28 days, compared to skin thickness without application of the topical composition.

In yet another aspect, skin irritation is reduced and/or prevented after administration of the topical composition of the present invention compared to skin irritation without administration of the topical composition.

In yet another aspect, skin redness is reduced and/or prevented after administration of the topical composition compared to skin redness without administration of the topical composition.

In a preferred embodiment, skin redness is reduced, indicated by a reduction of the hue and/or color saturation on the red-green axis a*, which is at least 4.5%, preferably at least 10%, more preferably at least 15%, most preferably at least 20% greater after administration of the topical composition for at least 1 day, preferably 2 days, compared to the reduction of the hue and/or color saturation on the red-green axis a* without administration of the topical composition.

In another preferred embodiment, skin redness is prevented indicated by an increase of the hue and/or color saturation on the red-green axis a*, which is at least 6% less, preferably at least 20% less, more preferably at least 25% less, most preferably at least 28% less after administration of the topical composition together with sodium dodecyl sulphate for at least 1 day, preferably at least 7 days, compared to the increase of the hue and/or color saturation on the red-green axis a* after administration of sodium dodecyl sulphate without administration of the topical composition.

In one embodiment of the present invention, the topical composition according to the present invention is a cosmetic topical composition. Accordingly, any salt in this topical composition is preferably a cosmetically acceptable salt. Further, any ester in this topical composition is preferably a cosmetically acceptable ester.

In a preferred embodiment, the cosmetic topical composition according to the present invention may further comprise an additional cosmetically active agent.

What is meant by a “cosmetically active agent” is a compound that has a cosmetic effect on the skin, hair, or nails, e.g., lightening agents, darkening agents such as self-tanning agents, anti-acne agents, shine control agents, anti-microbial agents, anti-inflammatory agents, anti-mycotic agents, ant-dandruff agents, anti-parasite agents, external analgesics, sunscreens, photoprotectors, antioxidants, keratolytic agents, moisturizers, nutrients, vitamins, energy enhancers, anti-perspiration agents, astringents, deodorants, hair removers, firming agents, anti-callous agents, and agents for hair, nail, and/or skin conditioning.

Preferably, the cosmetically active agent is selected from, but not limited to, the group consisting of benzoyl peroxide, sulfur, resorcinol, D-panthenol, hydroquinone, octyl methoxycinnimate, titanium dioxide, octyl salicylate, homosalate, avobenzone, polyphenolics, carotenoids, free radical scavengers, spin traps, retinoids such as retinol and retinyl palmitate, ceramides, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, peptides such as those disclosed in PCT Patent Application WO00/15188, vitamins, acetyl-coenzyme A, niacin, riboflavin, thiamin, ribose, azoles such as miconazole and ketoconazole, zinc pyrithione, coal tar, triclosan, electron transporters such as NADH and FADH2, and other botanical extracts such as aloe vera, and derivatives and mixtures thereof. The cosmetically active agent will typically be present in the topical composition of the invention in an amount of from about 0.001% to about 20% by weight of the topical composition, e.g., about 0.01% to about 10% such as about 0.1% to about 5%.

In one aspect, the cosmetic topical composition according to the present invention is suitable for use in improving or maintaining the biomechanical properties of the skin. Preferably, the biomechanical properties are selected from skin firmness, skin elasticity, and skin fatigue resistance. More preferably, the biomechanical properties of the skin are improved towards the firm-elastic optimum.

In another aspect, the cosmetic topical composition according to the present invention is suitable for use in decreasing wrinkle depth, wrinkle area and/or wrinkle volume. Preferably, the cosmetic topical composition is suitable for use in decreasing wrinkle depth, wrinkle area, and/or wrinkle volume of wrinkles of skin, preferably of inner side of forearms or face, more preferably in Crow's feet.

In another aspect, the cosmetic topical composition according to the present invention is suitable for use in protecting skin from oxidative stress, preferably photooxidative stress. Preferably, the cosmetic topical composition is suitable for use in protecting skin from photooxidative stress induced by UV radiation, preferably UVA radiation, by increasing the elimination of free radicals formed under UV radiation, preferably UVA radiation.

In another aspect, the cosmetic topical composition according to the present invention is suitable for use in preventing or reducing skin roughness.

In yet another aspect, the cosmetic topical composition according to the present invention is suitable for use in maintaining or improving skin hydration.

In yet another aspect, the cosmetic topical composition according to the present invention is suitable for use in maintaining or improving skin thickness.

Thus, the cosmetic topical composition according to the present invention is suitable for use in
a) maintaining or improving the biomechanical properties of skin,
b) decreasing wrinkle depth, wrinkle area and/or wrinkle volume,
c) protecting skin from oxidative stress,
d) preventing or reducing skin roughness,
e) maintaining or improving skin hydration, and/or
f) maintaining or improving skin thickness.

In another aspect, the cosmetic topical composition according to the present invention is suitable for use in improving and/or maintaining the collagen structure of the skin. Preferably, the cosmetic topical composition is suitable for use in modulating, preferably maintaining and/or increasing, the formation of collagen in the skin.

The present invention is also directed to the use of the cosmetic topical composition of the present invention for improving or maintaining the biomechanical properties of the skin. Preferably, the biomechanical properties are selected from skin firmness, skin elasticity, and skin fatigue resistance. More preferably, the biomechanical properties of the skin are improved towards the firm-elastic optimum.

Further, the present invention is also directed to the use of the cosmetic topical composition of the present invention for decreasing wrinkle depth, wrinkle area and/or wrinkle volume. Preferably, the present invention is directed to the use of the cosmetic topical composition for decreasing wrinkle depth, wrinkle area, and/or wrinkle volume of wrinkles of skin, preferably of inner side of forearms or face, more preferably in Crow's feet.

Still further, the present invention is directed to the use of the cosmetic topical composition of the present invention for protecting skin from oxidative stress, preferably photooxidative stress. Preferably, the present invention is directed to the use of the cosmetic topical composition of the present invention for protecting the skin from photooxidative stress induced by UV radiation, preferably UVA radiation, by increasing the elimination of free radicals formed under UV radiation, preferably UVA radiation.

Still further, the present invention is directed to the use of the cosmetic topical composition of the present invention for preventing or reducing skin roughness.

Still further, the present invention is directed to the use of the cosmetic topical composition of the present invention for maintaining or improving skin hydration.

Still further, the present invention is directed to the use of the cosmetic topical composition of the present invention for maintaining or improving skin thickness.

Thus, the present invention is directed to the use of the cosmetic topical composition of the present invention for
a) maintaining or improving the biomechanical properties of skin,
b) decreasing wrinkle depth, wrinkle area and/or wrinkle volume,
c) protecting skin from oxidative stress,
d) preventing or reducing skin roughness,
e) maintaining or improving skin hydration, and/or
f) maintaining or improving skin thickness.

Furthermore, the present invention is directed to the use of the cosmetic topical composition of the present invention for improving and/or maintaining the collagen structure of the skin. Preferably, the present invention is directed to the use of the cosmetic topical composition of the present invention for modulating, preferably maintaining and/or increasing, the formation of collagen in the skin.

In one aspect, the present invention is also directed to a cosmetic method for improving or maintaining the biomechanical properties of the skin comprising contacting the cosmetic topical composition according to the present invention with skin. Preferably, the biomechanical properties are selected from skin firmness, skin elasticity, and skin fatigue resistance. More preferably, the biomechanical properties of the skin are improved towards the firm-elastic optimum.

In another aspect, the present invention is directed to a cosmetic method for decreasing wrinkle depth, wrinkle area, and/or wrinkle volume comprising contacting the cosmetic topical composition according to the present invention with skin. In a preferred embodiment, the present invention is directed to a cosmetic method for decreasing wrinkle depth, area, and volume of wrinkles of skin, preferably of inner side of forearms or face, more preferably Crow's feet.

In another aspect, the present invention is directed to a cosmetic method for protecting skin from oxidative stress, preferably photooxidative stress, comprising contacting the cosmetic topical composition according to the present invention with skin. Preferably, the skin is protected from photooxidative stress induced by UV radiation, preferably UVA radiation, by increasing the elimination of free radicals.

In another aspect, the present invention is directed to a cosmetic method for preventing or reducing skin roughness comprising contacting the cosmetic topical composition according to the present invention with skin.

In another aspect, the present invention is directed to a cosmetic method for maintaining or improving skin hydration comprising contacting the cosmetic topical composition according to the present invention with skin.

In another aspect, the present invention is directed to a cosmetic method for maintaining or improving skin thickness comprising contacting the cosmetic topical composition according to the present invention with skin.

Thus, the present invention is directed to a cosmetic method for
a) maintaining or improving the biomechanical properties of skin,
b) decreasing wrinkle depth, wrinkle area and/or volume,
c) protecting skin from oxidative stress,
d) preventing or reducing skin roughness,
e) maintaining or improving skin hydration, and/or
f) maintaining or improving skin thickness
   comprising contacting the cosmetic topical composition according to the present invention with skin.

In another aspect, the present invention is directed to a cosmetic method for improving and/or maintaining the collagen structure of the skin comprising contacting the cosmetic topical composition according to the present with skin. Preferably, the method is suitable for modulating, preferably maintaining and/or increasing, the formation of collagen in the skin.

In one embodiment of the present invention, the topical composition according to the present invention is a pharmaceutical topical composition. Accordingly, any salt in the topical composition is preferably a pharmaceutically acceptable salt. Further, any ester in the topical composition is preferably a pharmaceutically acceptable ester.

In a preferred embodiment, the pharmaceutical topical composition according to the present invention may further comprise an additional pharmaceutically active agent.

Preferably, a “pharamceutically active agent” is a compound that has a therapeutic effect on the skin, e.g. HmG CoA reductase, an anti-inflammatory agent, and/or a corticosteroid.

In one aspect, the pharmaceutical topical composition according to the present invention is suitable for use in treating or preventing skin irritation.

In yet another aspect, the pharmaceutical topical composition according to the present invention is suitable for use in treating or preventing neurodermitis.

In another aspect, the pharmaceutical topical composition according to the present invention is suitable for use in treating or preventing skin sensations preferably selected from the group consisting of stinging, burning, tingling, itching, tickling, skin tightness or any combinations of the foregoing.

In another aspect, the pharmaceutical topical composition according to the present invention is suitable for use in treating or preventing skin redness.

Thus, the pharmaceutical topical composition according to the present invention is suitable for use in
a) treating or preventing skin irritation,
b) treating or preventing neurodermitis,
c) treating or preventing skin sensations selected from the group consisting of stinging, burning, tingling, itching, tickling, skin tightness or any combinations of the foregoing, and/or
d) treating or preventing skin redness.

In one aspect, the present invention is also directed to a method for treating or preventing skin irritation comprising contacting the pharmaceutical topical composition according to the present invention with skin.

In yet another aspect, the present invention is directed to a method for treating or preventing neurodermitis, comprising contacting the pharmaceutical topical composition to the present invention with skin.

In another aspect, the present invention is directed to a method for treating or preventing skin sensations preferably selected from the group consisting of stinging, burning, tingling, itching, tickling, skin tightness or any combinations of the foregoing, comprising contacting the pharmaceutical topical composition according to the present invention with skin.

In yet another aspect, the present invention is directed to a method for treating or preventing skin redness, comprising contacting the pharmaceutical topical composition according to the present invention with skin.

Thus, the present invention is directed to a method for
a) treating or preventing skin irritation,
b) treating or preventing neurodermitis,
c) treating or preventing skin sensations selected from the group consisting of stinging, burning, tingling, itching, tickling, skin tightness or any combinations of the foregoing,
d) treating or preventing skin changes, preferably skin redness, and/or
   comprising contacting the pharmaceutical topical composition according to the present invention with skin.

The invention further comprises the following preferred embodiments:

1. A pharmaceutical composition for topical administration comprising at least one angiotensin II receptor antagonist in a subtherapeutic dose and at least one antioxidant or any mixtures thereof for use in improving and/or maintaining collagen structure of apparently healthy skin.

2. The pharmaceutical composition for topical administration according to item 1, wherein angiotensin II receptor antagonist is selected from the group consisting of azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof.

3. The pharmaceutical composition for topical administration according to any of the previous items, wherein angiotensin II receptor antagonist is valsartan.

4. The pharmaceutical composition for topical administration according to any previous items, wherein angiotensin II receptor antagonist is present in an amount of between 1 and 10 w %, preferably between 1 and 5 w % and more preferably between 1 and 3 w % relative to the total weight of the composition.

5. The pharmaceutical composition for topical administration according to any of the previous items, wherein antioxidant is selected from the group consisting of beta-carotene, ascorbic acid, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole and any pharmaceutically acceptable salts thereof and/or any analogues thereof and preferably beta-carotene, ascorbic acid, tocopherol, tocopherol acetate, sodium ascorbyl phosphate, retinyl palmitate, coenzyme Q10 and resveratrol and any pharmaceutically acceptable salts thereof and/or any analogue thereof.

6. The pharmaceutical composition for topical administration according to any of the previous items, wherein comprising antioxidants tocopherol, tocopherol acetate, sodium ascorbyl phosphate and retinyl palmitate.

7. The pharmaceutical composition for topical administration according to any of the previous items, wherein antioxidant is present in the efficient amount from 2 to 20 w %, preferably from 3 to 15 w % and more preferably from 4 to 10 w % relative to the total weight of the composition.

8. The pharmaceutical composition for topical administration according to any of the previous items, wherein the composition is applied to body surfaces such as the skin or mucous membranes.

9. The pharmaceutical composition for topical administration according to any of the previous items, wherein the composition is in the form of liquids selected from the group consisting of, emulsions, suspensions, solutions, sprays or shampoos, semi-solids selected from the group consisting of collodions, foams, ointments, pastes, creams or gels, or solids selected from the group consisting of powders, aerosols, patches, gauzes, compress, tapes, sticks or tablets for topical application, preferably in the form of cream, lotion or skin patch, more preferably in the form of cream.

10. The pharmaceutical composition for topical administration according to any one of the previous items, further comprising at least one cosmetic acceptable ingredients.

11. A pharmaceutical composition for topical administration comprising at least one angiotensin II receptor antagonist selected from the group consisting of losartan, telmisartan, azilsartan, candesartan and valsartan and any pharmaceutically acceptable salts or esters thereof in an amount between 1 and 10 w %, preferably between 1 and 5 w % and more preferably between 1 and 3 w % relative to the total weight of the composition and at least one antioxidant selected from the group consisting of beta-carotene, ascorbic acid, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole and any pharmaceutically acceptable salts thereof and/or any analogues thereof or any mixtures thereof for use in improving and/or maintaining collagen structure of apparently healthy skin.

12. A pharmaceutical composition for topical administration comprising at least one angiotensin II receptor antagonist selected from the group consisting of losartan and valsartan and any pharmaceutically acceptable salts or esters thereof in an amount between 1 and 10 w %, preferably between 1 and 5 w % and more preferably between 1 and 3 w % relative to the total weight of the composition and at least one antioxidant selected from the group consisting of tocopherol, tocopherol acetate, sodium ascorbyl phosphate, retinyl palmitate or any mixtures thereof for use in improving and/or maintaining collagen structure of apparently healthy skin.

The present invention is illustrated in further detail with reference to the following examples. However, the scope of the present invention is not limited to these examples.

### Example 1

TABLE 1
List of ingredients of three tested products:

| | Placebo | | Active |
|---|---|---|---|
| | cream | Active cream + ACE | cream + ACE + X |
| angiotensin II | / | / | Losartan potassium |
| receptor | | | (20 mg) |
| antagonist | | | |
| antioxidant | / | Vitamin A (50 mg) | Vitamin A (50 mg) |
| | / | Vitamin C (50 mg) | Vitamin C (50 mg) |
| | / | Vitamin E (50 mg) | Vitamin E (50 mg) |
| Other | / | 2-hydroxy-4- | 2-hydroxy-4- |
| ingredients | | methoxybenzophenone | methoxybenzo- |
| | | (25 mg) | phenone |
| | | | (25 mg) |
| | / | 1-phenyl-3(4′- | 1-phenyl-3(4′- |
| | | isopropyl- | isopropyl- |
| | | phenyl)propane- | phenyl)propane- |
| | | 1,3-dione (25 mg) | 1,3-dione (25 mg) |
| | / | Macrogol stearate | Macrogol stearate |
| | | (100 mg) | (100 mg) |
| | / | Cetylstearyl alcohol | Cetylstearyl alcohol |
| | | (100 mg) | (100 mg) |
| | / | 1,2-propandiole | 1,2-propandiole |
| | | (100 mg) | (100 mg) |
| | Vaseline | Vaseline (white) | Vaseline (white) |
| | (white) | (500 mg) | (480 mg) |
| | (1000 mg) | | |

The three tested compositions were prepared by the method known from the state of the art.

### Example 1a

### Evaluation of the Efficacy in View of Biomechanical Properties

TABLE 2
Description of the evaluation

| | |
|---|---|
| Subjects | Number: 20 |
| | Age: 37-63 years (average: 44.2) |
| | Sex: female |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Products tested | Active cream ACE |
| | Active cream ACE + X |
| Region tested | inner side of forearms |
| | Crow's feet |
| Application | Duration: 42 days |
| | Frequency: twice daily |
| Evaluation method | Descriptive statistics (average, median, minimum, |
| | maximum, variance, standard error, standard deviation); |
| | ANOVA |
| | Tukey HSD |
| | Wilcoxon Rank Test |
| Design of study | Day 0: |
| | Determination of the parameters in the test areas |
| | First test product application |
| | Day 21: |
| | Determination of the parameters 8-12 hours |
| | following the last daily test product application |
| | Day 42: |
| | Determination of the parameters 8-12 hours |
| | following the last daily test product application |
| | The subjects used the test products (approximately |
| | 2 mg/cm²) twice daily in the morning and in the evening. |
| Test parameters | Determination of skin firmness and elasticity by means of |
| | Cutometer MPA 580 (Courage & Khazaka GmbH, |
| | Cologne; S/N 41032492 tube: S/N 04317556) |
| Measurement | The measurement is based on the vacuum-suction |
| | principle. By applying a constant negative pressure for a |
| | given period of time, skin is drawn into a hollow tube with |
| | an orifice of 2 mm in diameter. Then, at normal air |
| | pressure, skin is allowed to retract. The penetration depth |
| | of the skin into tube is recorded optically without friction |
| | and without mechanical influence. |
| | To increase accuracy and to capture information on the |
| | properties of skin under repeated external stress, the |
| | cycle is repeated several times and parameters selected |
| | for evaluation are based on areas rather than individual |
| | measurement points. |
| Expression of | The delineation of the areas is based on the fitted |
| results | logarithmic envelope curves of the minimum and |
| | maximum extensions according to the equation: |
| | $y = \frac{ln x + b}{a}$ |
| | X means repetions |
| | b means maximum amplitude |
| | a means minimum amplitude |
| | The study was conducted with 20 successive |
| | measurement cycles, 1 second suction, 1 second |
| | retraction, with a 450 mbar vacuum. |
| Parameters to | Skin firmness is assessed by the parameter F4 which |
| assess changes in | means the area below the approximated envelope |
| skin firmness | function of the maximum extensions. |
| | A decrease in F4 corresponds to an increase in skin |
| | firmness. |
| Parameters to | Skin elasticity is assessed by the ration F3/F4. The larger |
| assess changes in | F3 (which means area between minimum and maximum |
| skin elasticity | envelope) in comparison to F4, the larger are the |
| | restoring forces and the smaller is the remaining residual |
| | deformation. |
| | The closer the resulting value is to 1, the more elastic is |
| | skin. |
| Calculation of | The calculation of the parameters was conducted by |
| parameters | WinCT (Courage & Khazaka GmbH, Cologne, Germany) |

TABLE 3
Increase in skin firmness relative to initial conditions [%]

| | After 21 days | | | | | |
|---|---|---|---|---|---|---|
| | | | Active | After 42 days | | |
| | | Active | cream + | | Active | Active |
| | | cream + | ACE + | | cream + | cream + |
| | untreated | ACE | X | untreated | ACE | ACE + X |
| 1 | 9.5 | −10.9 | −15.1 | 8.7 | −15.4 | −2.7 |
| 2 | 8.8 | −5.6 | −11.7 | 20.3 | −13.9 | −31.0 |
| 3 | 8.7 | −5.0 | −6.0 | 2.2 | −17.1 | 1.0 |
| 4 | −5.4 | −10.1 | −5.3 | −4.8 | −24.6 | −24.7 |
| 5 | −12.6 | 11.6 | 5.2 | 14.0 | −10.9 | −22.6 |
| 6 | −6.3 | −6.0 | −8.2 | −6.1 | −18.6 | −21.9 |
| 7 | −3.8 | −12.1 | −13.5 | 2.9 | −22.9 | −34.2 |
| 8 | −4.0 | −7.7 | −10.2 | 1.7 | −12.7 | 2.8 |
| 9 | 4.8 | −3.7 | −24.1 | 4.3 | −8.6 | −26.2 |
| 10 | 17.7 | −17.6 | −8.6 | 7.8 | −24.8 | 11.3 |
| 11 | 0.4 | −7.1 | 9.6 | −3.0 | −20.6 | −14.4 |
| 12 | −0.4 | −7.0 | −6.5 | 5.7 | −5.7 | −0.9 |
| 13 | −0.1 | −18.7 | −15.0 | −0.7 | 11.1 | −20.9 |
| 14 | 5.9 | −14.9 | −14.4 | −3.5 | −11.8 | −2.9 |
| 15 | 16.3 | −5.6 | −3.0 | 6.8 | 8.7 | −8.3 |
| 16 | −13.5 | 10.6 | 3.8 | −12.9 | 1.3 | −29.6 |
| 17 | 3.1 | −6.3 | −12.5 | 7.8 | −34.3 | −33.2 |
| 18 | 17.6 | −7.0 | 3.8 | 6.8 | −7.5 | −9.2 |
| 19 | 13.2 | 1.8 | −17.4 | 6.4 | 7.3 | −17.8 |
| 20 | 2.7 | −3.0 | −6.0 | 15.5 | −9.4 | −14.2 |
| Average | 3.1 | 6.2 | 7.8 | 4.0 | 11.5 | 15.0 |
| S.D. | 9.3 | 7.7 | 8.5 | 7.9 | 11.9 | 13.3 |
| Median | 2.9 | −6.6 | −8.4 | 5.0 | −12.3 | −16.1 |
| Impr.* | — | 85 | 80 | — | 80 | 85 |

*% of subjects with relative improvement in test area as compared to initial condition and corrected by changes in untreated area

The tested product (Active cream+ACE+X) was found to statistically significantly increase skin firmness. The respective percentage changes as compared to the initial condition and with regard to the changes in the untreated area are shown in FIG. 1.

TABLE 4
Increase in skin elasticity relative to initial conditions [%]

| | After 21 days | | | | | |
|---|---|---|---|---|---|---|
| | | | Active | After 42 days | | |
| | | Active | cream + | | Active | Active |
| | | cream + | ACE + | | cream + | cream + |
| | untreated | ACE | X | untreated | ACE | ACE + X |
| 1 | 9.7 | 12.9 | 14.1 | 4.3 | 18.0 | 26.4 |
| 2 | −0.1 | 10.0 | 19.7 | 0.6 | 17.3 | 21.3 |
| 3 | −4.4 | 13.8 | 4.4 | 11.6 | −1.1 | −2.9 |
| 4 | −3.1 | 19.2 | 27.8 | 7.6 | 18.5 | 22.2 |
| 5 | −3.5 | 11.6 | 8.3 | −4.3 | 11.5 | 14.7 |
| 6 | 15.3 | −9.7 | 5.4 | −5.1 | 0.4 | 17.6 |
| 7 | 10.7 | 2.6 | −7.3 | 4.4 | 17.8 | 3.6 |
| 8 | 12.3 | −7.3 | −14.9 | 0.6 | 27.8 | 2.0 |
| 9 | 10.7 | −10.8 | −9.4 | 8.3 | −8.1 | −10.9 |
| 10 | −2.0 | −3.0 | −0.6 | 4.1 | −7.8 | 19.0 |
| 11 | 8.8 | −4.3 | −9.9 | 5.7 | −6.3 | 5.7 |
| 12 | 12.7 | 18.6 | 34.0 | 20.7 | −2.4 | 21.2 |
| 13 | −2.1 | 13.6 | 11.3 | 2.8 | −5.5 | 9.3 |
| 14 | 10.1 | 6.1 | −3.7 | −1.1 | 6.6 | 12.6 |
| 15 | −10.0 | 28.3 | 23.7 | −14.5 | 27.7 | 25.0 |
| 16 | −3.3 | 17.5 | 10.8 | 1.3 | 4.9 | 7.3 |
| 17 | 5.8 | 7.5 | −2.0 | 5.8 | 14.6 | −0.4 |
| 18 | 7.0 | −3.8 | 5.5 | 10.5 | −0.1 | 15.1 |
| 19 | −2.5 | −2.5 | 14.2 | 5.4 | −0.9 | −2.2 |
| 20 | 0.0 | −0.6 | 5.2 | −5.8 | 4.0 | 10.9 |
| Average | 3.6 | 6.0 | 6.8 | 3.1 | 6.9 | 10.9 |
| S.D. | 7.4 | 11.0 | 13.1 | 7.4 | 11.5 | 10.4 |
| Median | 2.9 | 6.8 | 5.5 | 4.2 | 4.5 | 11.7 |
| Impr.* | — | 60 | 65 | — | 60 | 80 |

*% of subjects with relative improvement in test area as compared to initial condition and corrected by changes in untreated area

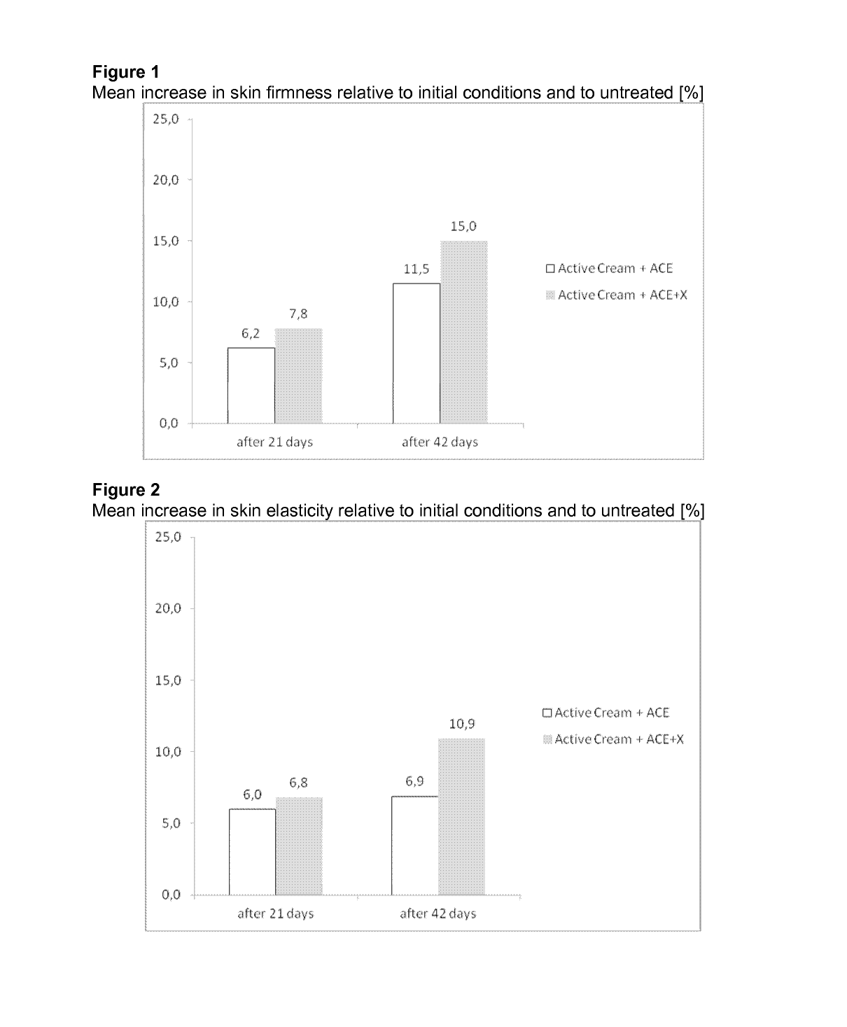

The tested product (Active cream+ACE+X) was found to statistically significantly increase skin elasticity. The respective percentage changes as compared to the initial condition and with regard to the changes in the untreated area are shown in FIG. 2.

The product marked as Active cream+ACE+X and containing losartan was found to increase skin firmness and at the same time skin elasticity. Overall, it induces a change in the biomechanical properties of skin towards the firm-elastic optimum.

### Example 1b

### Evaluation of the Efficacy in View of Wrinkle Depth

TABLE 5
Description of the evaluation

| | |
|---|---|
| Subjects | Number: 20 |
| | Age: 37-63 years (average: 44.2) |
| | Sex: female |
| | Clinically healthy subjects that were instructed not to use |
| | any topical preparations on the test areas starting from |
| | seven days prior to testing and until the end of the test. |
| | No incompatibility was observed in any of the subjects. |
| Products tested | Active cream ACE |
| | Active cream ACE + X |
| Region tested | inner side of forearms |
| | Crow's feet |
| Application | Duration: 42 days |
| | Frequency: twice daily |
| Evaluation | Descriptive statistics (average, median, minimum, |
| method | maximum, variance, standard error, standard deviation); |
| | ANOVA |
| | Tukey HSD |
| | Wilcoxon Rank Test |
| Design of study | Day 0: |
| | Determination of the parameters in the test areas |
| | First test product application |
| | Day 21 |
| | Determination of the parameters 8-12 hours |
| | following the last daily test product application |
| | Day 42: |
| | Determination of the parameters 8-12 hours |
| | following the last daily test product application |
| | The subjects used the test products (approximately |
| | 2 mg/cm²) twice daily in the morning and in the evening. |
| Test parameters | Determination of wrinkle depth by means of PRIMOS ® |
| | 4 (GFMeβtechnik GmbH, Teltow, Germany) |
| Measurement | The crow feet area is recorded as a 3D topography using |
| | the PRIMOS system. |
| Expression of | Wrinkle depth was assessed by means of the parameter |
| results | Rₘₐₓ that is defined as the maximum vertical distance |
| | from the highest peak to the lowest valley of five |
| | segments of equal length. To mitigate locational effects, |
| | the evaluation was conducted using the arithmetic |
| | average of Rₘₐₓ from 10 parallel cuts. |

TABLE 6
Decrease in wrinkle depth relative to initial conditions [%]

| | |
|---|---|
| After 21 days | After 42 days |

| | Active | Active | Active | Active |
|---|---|---|---|---|
| | cream + | cream + | cream + | cream + |
| | ACE | ACE + X | ACE | ACE + X |
| | A | B | A | B |
| 1 | −14.8 | 1.2 | −28.9 | −18.9 |
| 2 | 11.4 | 1.2 | −2.8 | −1.8 |
| 3 | −16.6 | −26.4 | −29.4 | −27.9 |
| 4 | 11.0 | −8.0 | −17.5 | 0.9 |
| 5 | −5.1 | 0.4 | −10.5 | −4.1 |
| 6 | 7.5 | −21.7 | 9.9 | −28.2 |
| 7 | −17.5 | −23.6 | −30.9 | −20.1 |
| 8 | −13.0 | 0.1 | −16.3 | −17.5 |
| 9 | −1.1 | −17.0 | −5.9 | −23.0 |
| 10 | −18.0 | −29.4 | −21.5 | −28.8 |
| 11 | −33.9 | 2.9 | −16.4 | −0.2 |
| 12 | −2.7 | −6.8 | −6.2 | −7.9 |
| 13 | −26.4 | −11.2 | 0.9 | −26.9 |
| 14 | −34.4 | −22.8 | −17.7 | −29.9 |
| 15 | 0.9 | −20.9 | 6.0 | −15.2 |
| 16 | −3.2 | 10.2 | −7.6 | 5.5 |
| 17 | −23.7 | −18.1 | −33.7 | −33.4 |
| 18 | −15.5 | −6.8 | −31.9 | −9.8 |
| 19 | −12.9 | 3.6 | 1.1 | −5.0 |
| 20 | −8.0 | −18.9 | −14.6 | −15.0 |
| Average | 10.8 | 10.6 | 13.7 | 15.4 |
| S.D. | 13.3 | 11.9 | 13.1 | 11.9 |
| Median | −13.0 | −9.6 | −15.5 | −16.3 |
| Impr.* | 80 | 65 | 80 | 90 |

*% of subjects with relative improvement in test area as compared to initial condition and corrected by changes in untreated area

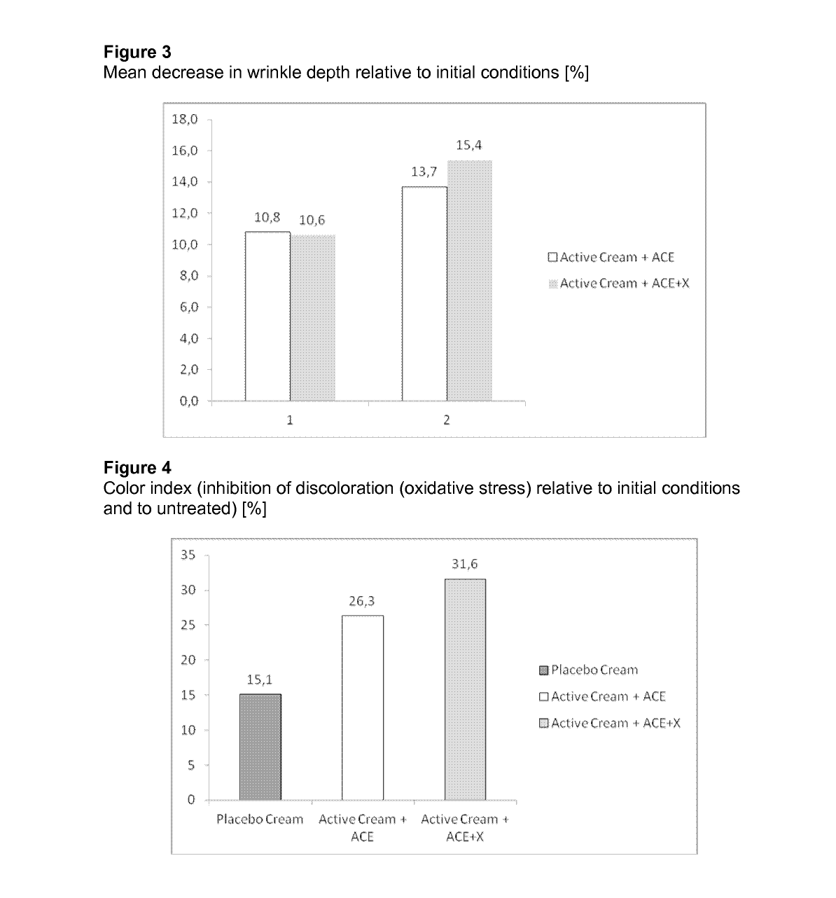

The tested product (Active cream+ACE+X) was found to statistically significantly decrease wrinkle depth. The respective percentage changes as compared to the initial condition are shown in FIG. 3.

### Example 1c

### Evaluation of the Efficacy Against Photooxidative Stress in Skin Induced by UVA-Irradiation

TABLE 7
Description of the evaluation

| | |
|---|---|
| Subjects | Number: 10 |
| | Age: 35-60 years |
| | Sex: 4 female, 6 male |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Formulations | Placebo |
| | Active cream ACE |
| | Active cream ACE + X |
| Region tested | inner side of forearms |
| Application | Single application |
| | On the inner side of the forearm of each subject |
| | symmetrically opposed areas were defined. The |
| | formulations were applied at a dose of approximately |
| | 2 mg/cm². 20 minutes after the application the β-carotene |
| | solution (5 mg/100 ml) in n-hexane (80 μl) was applied on |
| | each test site. 3 minutes later the test sites treated were |
| | irradiated (UVA: 10 Joule/cm²). Control area remained |
| | untreated. |
| Evaluation method | Change of color of β-carotene |
| Time of evaluation | Before and after UVA irradiation |
| Test method | β-carotene is a natural substance having orange-yellow |
| | color. Under the influence of UVA light it discolors as a |
| | result of a chemical process involving reactive oxygen |
| | species like singlet oxygen, the superoxide radical, |
| | hydrogen peroxide and the hydroxyl radical. |
| | If a product applied to the skin has antioxidant properties |
| | the color change in β-carotene is attenuated or inhibited. |
| Expression of | The results are expressed as the rate of inhibition relative |
| results | to the untreated area (Color index): |
| | $100 - \frac{{Sample}_{colorized} - {Sample}_{irradiated}}{{Control}_{colorized} - {Control}_{irradiated}} \times 100$ |
| | Colorized means color reading after coloration with β- |
| | carotene |
| | Irradiated means color reading after irradiation |
| | The color index in % corresponds directly to the efficacy |
| | against free radicals. |
| Measurement of | By means of Chromametry (Chromameter CR 300) in the |
| discoloration | L*a*b* colorimetry system (also designed as CIELAB |
| | system) |
| | L* represents brightness |
| | a* shows the position of the red-green axis |
| | b* shows the position on the yellow-blue axis |
| | Changes in the yellow color is shown by an increase or |
| | decrease in the b* value. |
| | Measurements were done according to the guidelines of |
| | the European Society of Contact Dermatitis (Fullerton et |
| | al., Guidelines for measurement of skin color and |
| | erythema. Contact Dermatitis 1996: 35; 1-10). |
| Biometry | The data were analyzed by ANOVA (within subject- |
| | design). In case of significant difference between tested |
| | areas they were compared with each other |
| | simultaneously by means of the Wilcoxon test. The 0.05 |
| | level was selected as the point of minimal acceptance of |
| | statistical significance. |

TABLE 8
Color Index (Inhibition of discoloration, i.e. oxidative stress,
relative to initial conditions) [%]

| | | Active | |
|---|---|---|---|
| | | cream + | Active cream + |
| | Placebo | ACE | ACE + X |
| 1 | 24.4 | 27.8 | 37.2 |
| 2 | 35.6 | 29.2 | 38.7 |
| 3 | 1.8 | 24.9 | 6.4 |
| 4 | 20.5 | 30.4 | 31.5 |
| 5 | 19.2 | 35.1 | 26.6 |
| 6 | 30.1 | 45.0 | 36.1 |
| 7 | −10.0 | 6.1 | 21.5 |
| 8 | 21.6 | 46.7 | 68.0 |
| 9 | 12.5 | 12.7 | 29.4 |
| 10 | −5.1 | 5.6 | 21.0 |
| Average | 15.1 | 26.3 | 31.6 |
| S.D. | 15.1 | 14.5 | 16.0 |
| Median | 19.8 | 28.5 | 30.5 |

The color index of the tested product (Active cream+ACE+X) was found to be statistically significantly larger than the placebo and Active cream without losartan. The respective percentage changes in discoloration in the treated area as percentages relative to the initial condition and to the color changes in the untreated area are presented in FIG. 4.

### Example 1d

### Evaluation of Efficacy in View of Skin Roughness

TABLE 9
Description of the evaluation

| | |
|---|---|
| Subjects | Number: 10 |
| | Age: 35-60 years |
| | Sex: 4 female, 6 male |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Formulations | Placebo |
| | Active cream ACE + X |
| Region tested | inner side of forearms |
| Application | Duration: 42 days |
| | Frequency: Twice daily |
| Evaluation | Descriptive statistics (average, median, minimum, |
| method | maximum, variance, standard error, standard deviation); |
| | ANOVA |
| | Tukey HSD |
| | Wilcoxon Rank Test |
| Design of study | Day 0: |
| | Determination of skin roughness in the test areas |
| | First test product application |
| | Day 21: |
| | Determination of skin roughness 8-12 hours |
| | following the last daily test product application |
| | Day 42: |
| | Determination of skin roughness 8-12 hours |
| | following the last daily test product application |
| | The subjects used the test products (approximately |
| | 2 mg/cm²) twice daily in the morning and in the evening. |
| Test method | Determination of skin roughness by means of |
| | PRIMOS ® 4 (GFMeβtechnik GmbH, Teltow, |
| | Germany) |
| Measurement | The tested area is recorded as a 3D topography using the |
| | PRIMOS system. |
| Expression of | Skin roughness was assessed by means of the |
| results | parameter R_{z} that is defined as the maximum vertical |
| | distance from the highest peak to the lowest valley of |
| | five segments of equal length. To mitigate locational |
| | effects, the evaluation was conducted using the |
| | arithmetic average of R_{z} from 10 parallel cuts. |

TABLE 10
PRIMOS readings for skin roughness (Rz, μm)

| | |
|---|---|
| After 21 days | After 42 days |

| | | Active | | Active |
|---|---|---|---|---|
| | | cream + | | cream + |
| | untreated | ACE + X | untreated | ACE + X |
| 1 | 22.21 | 17.94 | 15.48 | −7.93 |
| 2 | 19.33 | −14.36 | 9.84 | −23.87 |
| 3 | 14.94 | −19.13 | 11.49 | 0.55 |
| 4 | −20.76 | −31.26 | 18.54 | −16.98 |
| 5 | 4.38 | −20.96 | 10.61 | −3.57 |
| 6 | 9.73 | 13.54 | 2.61 | −34.35 |
| 7 | 6.08 | −33.76 | −4.18 | −44.65 |
| 8 | 19.85 | 12.12 | 12.13 | −1.10 |
| 9 | 22.30 | −38.57 | 9.11 | −41.88 |
| 10 | −3.74 | −23.99 | 13.63 | −19.53 |
| 11 | 27.82 | −19.85 | 31.96 | −20.52 |
| 12 | 6.65 | −64.58 | 36.15 | −8.14 |
| 13 | 31.78 | −10.04 | 5.61 | −27.37 |
| 14 | 7.55 | 12.19 | −15.26 | 21.00 |
| 15 | 7.11 | −5.26 | −9.26 | −17.96 |
| 16 | 21.91 | 29.86 | −11.04 | −49.97 |
| 17 | −7.62 | −35.53 | −8.83 | −30.68 |
| 18 | 21.77 | −10.85 | 27.76 | 10.97 |
| 19 | 2.21 | −16.82 | 24.40 | −19.40 |
| 20 | −20.00 | −16.02 | −28.19 | −23.67 |
| Average | 9.68 | −13.74 | 7.63 | −17.95 |
| S.D. | 14.61 | 22.60 | 16.61 | 18.20 |
| Median | 8.64 | −16.42 | 10.23 | −19.47 |

TABLE 11
Increase in skin smoothness, i.e. reduction of skin roughness,
relative to initial conditions [%]

| | |
|---|---|
| After 21 days | After 42 days |

| | | Active | | Active |
|---|---|---|---|---|
| | | cream + | | cream + |
| | untreated | ACE + X | untreated | ACE + X |
| 1 | 11.2 | −2.7 | 7.8 | −11.5 |
| 2 | 12.0 | −19.3 | 6.1 | −18.0 |
| 3 | 8.6 | −19.4 | 6.6 | −6.3 |
| 4 | −10.4 | −3.1 | 9.3 | −16.6 |
| 5 | 2.3 | −16.0 | 5.5 | −7.9 |
| 6 | 5.9 | 1.6 | 1.6 | −20.6 |
| 7 | 3.0 | −21.7 | −2.1 | −21.9 |
| 8 | 11.7 | −5.7 | 7.1 | −7.7 |
| 9 | 12.7 | −30.9 | 5.2 | −24.9 |
| 10 | −1.6 | −9.9 | 5.9 | −15.3 |
| 11 | 14.9 | −25.4 | 17.1 | −28.0 |
| 12 | 2.7 | −28.7 | 14.9 | −18.1 |
| 13 | 15.7 | −20.2 | 2.8 | −14.9 |
| 14 | 3.9 | 3.1 | −8.0 | 20.1 |
| 15 | 3.7 | −6.3 | −4.8 | −3.8 |
| 16 | 11.9 | 3.8 | −6.0 | −20.3 |
| 17 | −3.6 | −21.3 | −4.2 | −17.3 |
| 18 | 12.0 | −17.5 | 15.3 | −9.8 |
| 19 | 1.1 | −8.9 | 12.5 | −21.5 |
| 20 | −11.8 | 1.3 | −16.6 | 1.1 |
| Average | 5.3 | −12.3 | 3.8 | −13.2 |
| S.D. | 7.9 | 11.0 | 8.6 | 10.8 |
| Median | 4.9 | −12.9 | 5.7 | −15.9 |
| Impr.* | — | 80 | — | 90 |

*% of subjects with relative improvement in test area as compared to initial condition and corrected by changes in untreated area

After both 21 and 42 days of treatment, a statistically significant (p<0.05) decrease in R_{z} was observed in product treated test areas compared to the changes in untreated area.

Thus, the tested product (Active cream+ACE+X) was found to statistically significantly increase skin smoothness, i.e. decrease skin roughness.

### Example 2

TABLE 12
List of ingredients of tested product:

| | Active cream + ACE + X |
|---|---|
| | (further marked as A) |
| angiotensin II | valsartan (2%) |
| receptor antagonist | |
| antioxidant | vitamin A palmitate, caprylic/capric triglyceride, |
| | vitamin E (0.3-0.5%) |
| | sodium ascorbyl phosphate (vitamin C) (0.8-2%) |
| | vitamin E acetate (4-6%), alcohol (4-6%) |
| other agents | cetearyl alcohol, cetearyl glucoside (4-6%) |
| | alcohol denat. (4-6%) |
| | polycarylamide, C₁₃₋₁₄ isoparaffin, laureth-7 (3-5%) |
| | glycerin (2-5%) |
| | dicaprylyl carbonate (2-4%) |
| | dicaprylyl ether (1-3.5%) |
| | propylheptyl caprylate (1-3%) |
| | dimethicone (1-3%) |
| | stearic acid (1-3%) |
| | cetyl alcohol (1-2.5%) |
| | stearyl acohol (1-2%) |
| | triethanolamine (0.8-2%) |
| | phenoxyethanol (0.9%) |
| | ethylhexylglycerin (0.1%) |
| | disodium EDTA (0.1-0.2%) |
| | perfume (0.4%) |
| | water ad 100% |

% means weight % relative to the total weight of composition

All the ingredients except valsartan, alcohol, polycarylamide, C₁₃₋₁₄ isoparaffin, laureth-7, triethanolamine, sodium ascorbyl phosphate and perfume are mixed in the reactor, heated to temperature 95° C., homogenised and cooled to 30° C. In the obtained emulsion triethanolamine is added, the mixture is homogenised and the obtained emulsion is heated to 50° C. when polycarylamide, C₁₃₋₁₄ isoparaffinand laureth-7 are added. After homogenization and addition of NaOH the obtained emulsion is cooled to 30° C. when sodium ascorbyl phosphate and perfume are added. When cooling to 15° C. valsartan and alcohol are added. After homogenization the obtained product is heated to 25° C.

### Example 2a

### Evaluation of the Efficacy in View of Biomechanical Properties

TABLE 13
Description of the evaluation

| | |
|---|---|
| Subjects | Number: 20 (+1 reserve subject) |
| | Age: 36-65 years (average: 48.2) |
| | Sex: female |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Product tested | Composition as defined in Table 9 |
| Region tested | inner side of forearms |
| | face (Crow's feet/temple) |
| Application | Duration: 28 days |
| | Frequency: twice daily |
| Evaluation method | Descriptive statistics (average, median, minimum, |
| | maximum, variance, standard error, standard deviation); |
| | Wilcoxon Rank Test |
| Design of study | Day 0: |
| | Determination of the parameters in the test areas |
| | First test product application |
| | Day 14: |
| | Determination of the parameters 8-12 hours |
| | following the last daily test product application |
| | Day 28: |
| | Determination of the parameters 8-12 hours |
| | following the last daily test product application |
| | The subjects used the test products (approximately |
| | 2 mg/cm²) twice daily in the morning and in the evening. |
| Test parameters | Determination of skin firmness and elasticity by means of |
| | Cutometer MPA 580 (Courage & Khazaka GmbH, |
| | Cologne; S/N 32041888; tube: S/N 04317556) |
| Measurement | The measurement is based on the vacuum-suction |
| | principle. By applying a constant negative pressure for a |
| | given period of time, skin is drawn into a hollow tube with |
| | an orifice of 2 mm in diameter. Then, at normal air |
| | pressure, skin is allowed to retract. The penetration depth |
| | of the skin into the tube is recorded optically without |
| | friction and without mechanical influence. |
| | To increase accuracy and to capture information on the |
| | properties of skin under repeated external stress, the |
| | cycle is repeated several times and parameters selected |
| | for evaluation are based on areas rather than individual |
| | measurement points. |
| Expression of | The delineation of the areas is based on the fitted |
| results | logarithmic envelope curves of the minimum and |
| | maximum extensions according to the equation: |
| | $y = \frac{ln x + b}{a}$ |
| | x means repetions |
| | Y means max. amplitude or min. amplitude |
| | b means maximum amplitude |
| | a means minimum amplitude |
| | The study was conducted with 20 successive |
| | measurement cycles, 1 second suction, 1 second |
| | retraction, with a 450 mbar vacuum. |
| Parameters to | Skin firmness is assessed by the parameter F4 which |
| assess changes in | means the area below the approximated envelope |
| skin firmness | function of the maximum extensions. |
| | A decrease in F4 corresponds to an increase in skin |
| | firmness. |
| Parameters to | Skin elasticity is assessed by the ratio F3/F4. The larger |
| assess changes in | F3 (which means area between minimum and maximum |
| skin elasticity | envelope) in comparison to F4, the larger are the |
| | restoring forces and the smaller is the remaining residual |
| | deformation. |
| | The closer the resulting value is to 1, the more elastic is |
| | skin. |
| Parameters to | Skin fatigue resistance is assessed by the parameter F2. |
| assess changes in | F2 is the area between the final extension and the |
| skin fatigue | approximated envelope function of the maximum |
| resistance | extensions. |
| | A decrease in F2 corresponds to an increase in fatigue |
| | resistance. |
| Calculation of | The calculation of the parameters was conducted by |
| parameters | WinCT (Courage & Khazaka GmbH, Cologne, Germany) |

TABLE 14
Cutometer readings for skin firmness (F4)

| | | |
|---|---|---|
| start | after 14 days | after 28 days |

| | untr. | A | untr. | A | untr. | A |
|---|---|---|---|---|---|---|
| 1 | 18.80 | 14.85 | 15.66 | 9.83 | 13.71 | 7.58 |
| 2 | 16.16 | 14.44 | 14.62 | 11.14 | 16.68 | 11.95 |
| 3 | 16.05 | 15.28 | 15.79 | 14.04 | 16.52 | 13.53 |
| 4 | 16.78 | 16.23 | 16.42 | 14.99 | 16.52 | 13.25 |
| 5 | 16.98 | 21.65 | 17.89 | 17.53 | 18.26 | 16.55 |
| 6 | 19.39 | 24.15 | 19.85 | 21.23 | 17.50 | 20.13 |
| 7 | 19.19 | 20.66 | 20.63 | 18.33 | 19.63 | 16.77 |
| 8 | 21.60 | 21.31 | 17.66 | 16.76 | 19.96 | 17.97 |
| 9 | 16.55 | 16.95 | 14.92 | 15.55 | 18.82 | 15.48 |
| 10 | 17.96 | 20.07 | 19.23 | 18.73 | 16.64 | 14.99 |
| 11 | 22.85 | 22.57 | 22.35 | 18.72 | 18.30 | 12.65 |
| 12 | 18.26 | 18.99 | 17.43 | 18.70 | 18.43 | 16.13 |
| 13 | 18.98 | 19.02 | 18.68 | 20.14 | 19.79 | 15.87 |
| 14 | 20.13 | 22.04 | 20.88 | 20.38 | 20.57 | 18.43 |
| 15 | 19.38 | 15.61 | 19.01 | 15.40 | 18.83 | 16.22 |
| 16 | 19.05 | 17.46 | 21.84 | 16.23 | 21.15 | 13.24 |
| 17 | 21.00 | 17.74 | 22.83 | 17.65 | 20.36 | 15.05 |
| 18 | 21.22 | 21.93 | 21.24 | 19.70 | 21.73 | 19.40 |
| 19 | 19.37 | 17.08 | 18.55 | 15.93 | 19.33 | 16.12 |
| 20 | 15.23 | 15.49 | 15.78 | 14.07 | 15.93 | 12.71 |
| Average | 18.75 | 18.68 | 18.56 | 16.75 | 18.43 | 15.20 |
| S.D. | 2.03 | 2.96 | 2.54 | 2.99 | 2.00 | 2.90 |
| Median | 19.02 | 18.36 | 18.62 | 17.15 | 18.63 | 15.67 |

untr. subjects not receiving A
A composition as defined in Table 12

TABLE 15
Increase in skin firmness relative to initial conditions and to
untreated (corrected Cutometer readings, F4) [%]

| | |
|---|---|
| after 14 days | after 28 days |

| | untr. | A | untr. | A |
|---|---|---|---|---|
| 1 | −16.7 | −17.1 | −27.1 | −21.9 |
| 2 | −9.5 | −13.4 | 3.3 | −20.6 |
| 3 | −1.6 | −6.5 | 2.9 | −14.4 |
| 4 | −2.2 | −5.5 | −1.6 | −16.7 |
| 5 | 5.4 | −24.4 | 7.6 | −31.1 |
| 6 | 2.3 | −14.4 | −9.7 | −6.9 |
| 7 | 7.5 | −18.8 | 2.3 | −21.1 |
| 8 | −18.2 | −3.1 | −7.6 | −8.1 |
| 9 | −9.8 | 1.6 | 13.7 | −22.4 |
| 10 | 7.1 | −13.8 | −7.4 | −17.9 |
| 11 | −2.2 | −14.9 | −19.9 | −24.1 |
| 12 | −4.5 | 3.0 | 0.9 | −16.0 |
| 13 | −1.6 | 7.5 | 4.3 | −20.8 |
| 14 | 3.7 | −11.3 | 2.2 | −18.6 |
| 15 | −1.9 | 0.6 | −2.9 | 6.8 |
| 16 | 14.7 | −21.7 | 11.0 | −35.2 |
| 17 | 8.7 | −9.2 | −3.1 | −12.1 |
| 18 | 0.1 | −10.3 | 2.4 | −13.9 |
| 19 | −4.2 | −2.6 | −0.2 | −5.5 |
| 20 | 3.6 | −12.7 | 4.6 | −22.5 |
| Average | −1.0 | −9.3 | −1.2 | −17.2 |
| S.D. | 8.2 | 8.6 | 9.6 | 9.3 |
| Median | −1.6 | −10.8 | 1.6 | −18.3 |
| Impr.* | — | 80 | — | 95 |

*% of subjects with relative improvement in test area as compared to initial condition and corrected by changes in untreated area
untr. subjects not receiving A
A composition as defined in Table 12

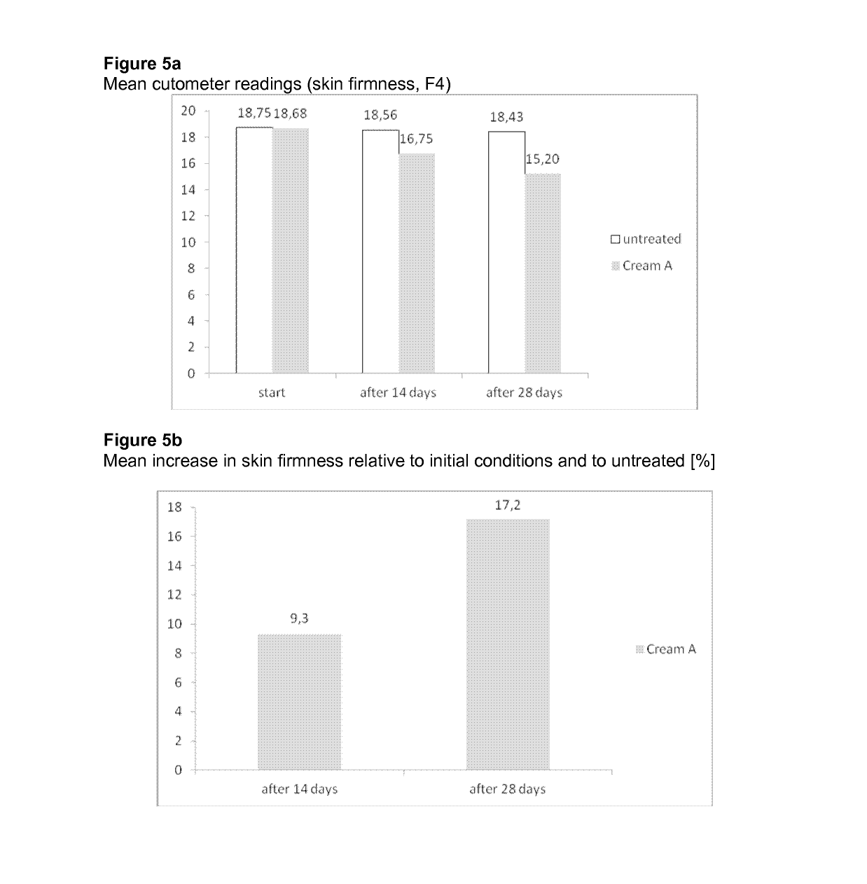

In assessing skin firmness, evaluated are changes in the parameter F4 in the treated area in comparison to the changes in the untreated area. A decrease in F4 corresponds to an increase in skin firmness. After both 14 and 28 days of treatment, a statistically significant (p<0.05) decrease in F4 was observed in the product treated test area as compared to the changes in the untreated area (FIG. 5a).

The test product was found to statistically significantly increase skin firmness; after 28 days of treatment a positive effect could be detected in 95% of the study participants. The respective percentage changes as compared to the initial condition and with regard of the changes in the untreated area are shown in FIG. 5b.

TABLE 16
Cutometer readings for skin elasticity (F3/F4)

| | | |
|---|---|---|
| start | after 14 days | after 28 days |

| | untr. | A | untr. | A | untr. | A |
|---|---|---|---|---|---|---|
| 1 | 0.73 | 0.78 | 0.75 | 0.86 | 0.79 | 0.94 |
| 2 | 0.50 | 0.53 | 0.52 | 0.58 | 0.53 | 0.63 |
| 3 | 0.76 | 0.81 | 0.70 | 0.72 | 0.68 | 0.80 |
| 4 | 0.62 | 0.71 | 0.66 | 0.75 | 0.68 | 0.76 |
| 5 | 0.65 | 0.68 | 0.71 | 0.75 | 0.67 | 0.70 |
| 6 | 0.65 | 0.66 | 0.60 | 0.66 | 0.62 | 0.70 |
| 7 | 0.75 | 0.69 | 0.71 | 0.74 | 0.68 | 0.74 |
| 8 | 0.69 | 0.75 | 0.65 | 0.65 | 0.71 | 0.70 |
| 9 | 0.73 | 0.71 | 0.76 | 0.74 | 0.70 | 0.72 |
| 10 | 0.80 | 0.73 | 0.83 | 0.79 | 0.73 | 0.79 |
| 11 | 0.65 | 0.62 | 0.50 | 0.60 | 0.60 | 0.71 |
| 12 | 0.45 | 0.40 | 0.39 | 0.45 | 0.39 | 0.47 |
| 13 | 0.67 | 0.65 | 0.65 | 0.72 | 0.69 | 0.73 |
| 14 | 0.72 | 0.72 | 0.74 | 0.80 | 0.73 | 0.84 |
| 15 | 0.60 | 0.60 | 0.63 | 0.72 | 0.63 | 0.75 |
| 16 | 0.77 | 0.75 | 0.81 | 0.82 | 0.81 | 0.80 |
| 17 | 0.47 | 0.61 | 0.46 | 0.62 | 0.49 | 0.60 |
| 18 | 0.65 | 0.63 | 0.61 | 0.70 | 0.68 | 0.75 |
| 19 | 0.72 | 0.73 | 0.79 | 0.83 | 0.74 | 0.86 |
| 20 | 0.75 | 0.79 | 0.77 | 0.79 | 0.73 | 0.82 |
| Average | 0.67 | 0.68 | 0.66 | 0.71 | 0.66 | 0.74 |
| S.D. | 0.10 | 0.10 | 0.12 | 0.10 | 0.10 | 0.10 |
| Median | 0.68 | 0.70 | 0.68 | 0.73 | 0.68 | 0.75 |

untr. subjects not receiving A
A composition as defined in Table 12

TABLE 17
Increase in skin elasticity relative to initial conditions and to
untreated (corrected Cutometer readings, F3/F4) [%]

| | |
|---|---|
| after 14 days | after 28 days |

| | untr. | A | untr. | A |
|---|---|---|---|---|
| 1 | 3.4 | 6.9 | 9.5 | 11.3 |
| 2 | 3.9 | 4.9 | 5.8 | 12.4 |
| 3 | −8.1 | −3.5 | −10.7 | 10.1 |
| 4 | 6.2 | −0.2 | 9.5 | −1.3 |
| 5 | 8.9 | 1.5 | 2.6 | 0.5 |
| 6 | −8.0 | 7.6 | −5.1 | 9.7 |
| 7 | −5.9 | 13.8 | −9.5 | 17.4 |
| 8 | −6.2 | −7.9 | 2.5 | −9.5 |
| 9 | 5.0 | −1.9 | −3.7 | 4.0 |
| 10 | 4.0 | 3.3 | −9.0 | 16.5 |
| 11 | −23.2 | 19.1 | −8.1 | 21.5 |
| 12 | −12.3 | 25.6 | −13.0 | 31.4 |
| 13 | −2.8 | 13.4 | 3.2 | 8.7 |
| 14 | 3.0 | 7.7 | 0.8 | 16.6 |
| 15 | 4.5 | 17.0 | 4.4 | 22.2 |
| 16 | 4.8 | 5.2 | 4.8 | 2.3 |
| 17 | −4.0 | 6.3 | 2.9 | −4.5 |
| 18 | −6.1 | 16.8 | 5.5 | 13.5 |
| 19 | 10.1 | 3.3 | 3.4 | 15.2 |
| 20 | 2.7 | −3.0 | −3.5 | 6.8 |
| Average | −1.0 | 6.8 | −0.4 | 10.2 |
| S.D. | 8.2 | 8.6 | 6.9 | 9.8 |
| Median | 2.9 | 5.7 | 2.6 | 10.7 |
| Impr.* | — | 75 | — | 85 |

*% of subjects with relative improvement in test area as compared to initial condition and corrected by changes in untreated area
untr. subjects not receiving A
A composition as defined in Table 12

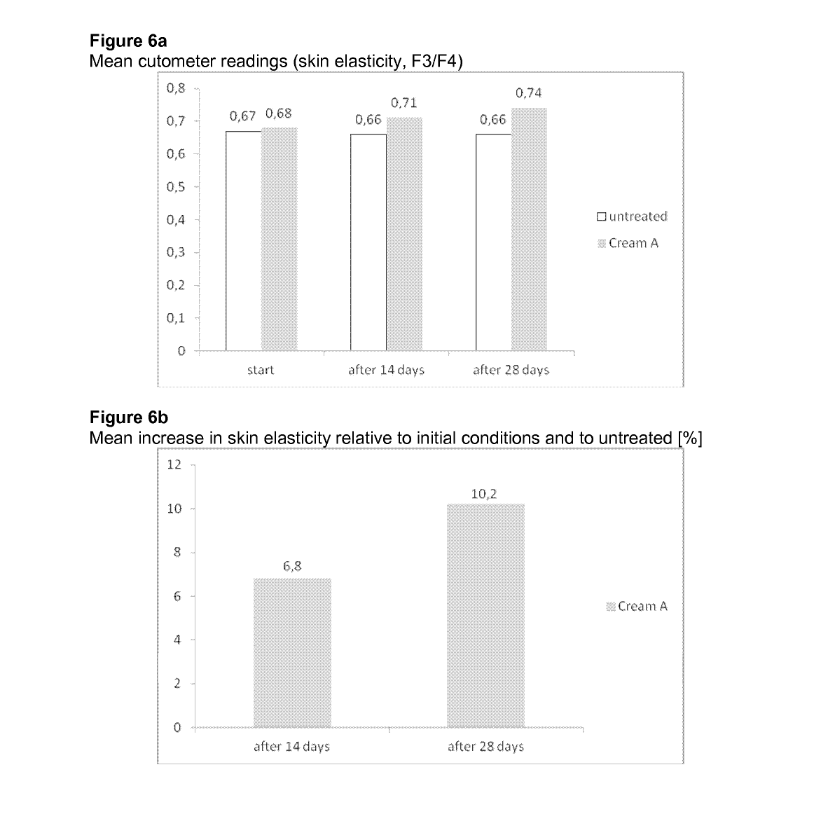

In assessing skin elasticity, evaluated are the changes in the fraction F3 divided by F4 in the treated area in comparison to the changes in the untreated area. The absolute changes by area and time point are shown FIG. 6a. An increase in F3/F4 corresponds to an increase in skin elasticity.

After both 14 and 28 days of treatment, a statistically significant (p<0.05) increase in F3/F4 was observed in the product treated test area as compared to the changes in the untreated area. The test product was found to statistically significantly increase skin elasticity; after 28 days of treatment a positive effect could be detected in 85% of the study participants. The respective percentage changes as compared to the initial condition and with regard of the changes in the untreated area are shown in FIG. 6b.

TABLE 18
Cutometer readings for skin fatigue resistance (F2)

| | | |
|---|---|---|
| start | after 14 days | after 28 days |

| | untr. | A | untr. | A | untr. | A |
|---|---|---|---|---|---|---|
| 1 | 1.36 | 1.27 | 1.39 | 1.25 | 1.29 | 1.15 |
| 2 | 1.23 | 1.33 | 1.30 | 1.35 | 1.36 | 1.43 |
| 3 | 1.26 | 1.21 | 1.10 | 1.22 | 1.29 | 1.19 |
| 4 | 1.25 | 1.52 | 1.25 | 1.34 | 1.28 | 1.47 |
| 5 | 1.86 | 1.85 | 1.78 | 1.66 | 1.62 | 1.72 |
| 6 | 1.28 | 1.29 | 1.49 | 1.08 | 1.34 | 1.06 |
| 7 | 0.91 | 1.11 | 1.09 | 1.09 | 1.10 | 1.14 |
| 8 | 1.41 | 1.54 | 1.34 | 1.43 | 1.18 | 1.37 |
| 9 | 1.55 | 1.84 | 1.55 | 1.14 | 1.65 | 1.18 |
| 10 | 0.99 | 1.44 | 0.91 | 0.78 | 1.01 | 0.77 |
| 11 | 1.29 | 1.73 | 1.33 | 1.44 | 1.27 | 1.66 |
| 12 | 1.71 | 1.71 | 1.65 | 1.67 | 1.65 | 1.64 |
| 13 | 2.01 | 1.64 | 2.15 | 1.76 | 2.28 | 1.99 |
| 14 | 1.15 | 0.90 | 1.07 | 0.82 | 1.33 | 0.95 |
| 15 | 1.07 | 0.86 | 0.96 | 0.61 | 0.99 | 0.66 |
| 16 | 1.22 | 1.16 | 0.86 | 1.10 | 1.27 | 1.13 |
| 17 | 1.76 | 1.45 | 1.59 | 1.20 | 1.72 | 1.24 |
| 18 | 1.77 | 1.93 | 1.46 | 1.52 | 1.44 | 1.57 |
| 19 | 1.61 | 1.55 | 1.66 | 1.43 | 1.61 | 0.98 |
| 20 | 1.25 | 1.37 | 1.37 | 1.33 | 1.23 | 1.10 |
| Average | 1.40 | 1.43 | 1.37 | 1.26 | 1.40 | 1.27 |
| S.D. | 0.30 | 0.30 | 0.32 | 0.30 | 0.30 | 0.33 |
| Median | 1.28 | 1.45 | 1.35 | 1.29 | 1.31 | 1.19 |

untr. subjects not receiving A
A composition as defined in Table 12

TABLE 19
Increase in skin fatigue resistance relative to initial conditions
and to untreated (corrected Cutometer readings, F2) [%]

| | |
|---|---|
| after 14 days | after 28 days |

| | untr. | A | untr. | A |
|---|---|---|---|---|
| 1 | 1.8 | −2.9 | −5.5 | −4.2 |
| 2 | 5.1 | −3.0 | 10.4 | −2.7 |
| 3 | −12.5 | 13.4 | 2.3 | −3.3 |
| 4 | 0.2 | −12.0 | 2.8 | −6.1 |
| 5 | −4.0 | −6.0 | −12.9 | 6.1 |
| 6 | 16.2 | −32.4 | 4.7 | −22.7 |
| 7 | 19.8 | −22.0 | 20.1 | −18.0 |
| 8 | −5.2 | −1.8 | −16.4 | 5.4 |
| 9 | 0.2 | −38.3 | 6.2 | −41.8 |
| 10 | −7.6 | −38.4 | 2.9 | −49.3 |
| 11 | 2.9 | −19.5 | −1.6 | −2.4 |
| 12 | −3.8 | 1.5 | −3.4 | −0.4 |
| 13 | 7.3 | 0.3 | 13.7 | 8.2 |
| 14 | −6.7 | −2.1 | 15.6 | −9.8 |
| 15 | −11.0 | −17.9 | −7.5 | −15.7 |
| 16 | −29.4 | 24.2 | 4.1 | −6.6 |
| 17 | −9.4 | −7.8 | −2.1 | −12.2 |
| 18 | −17.6 | −3.7 | −18.8 | 0.5 |
| 19 | 3.4 | −11.2 | 0.1 | −36.9 |
| 20 | 9.2 | −12.3 | −1.7 | −18.1 |
| Average | −2.1 | −9.6 | 0.6 | −11.5 |
| S.D. | 11.4 | 15.7 | 10.1 | 15.9 |
| Median | −1.8 | −6.9 | 1.2 | −6.4 |
| Impr.* | — | 80 | — | 80 |

*% of subjects with relative improvement in test area as compared to initial condition and corrected by changes in untreated area
untr. subjects not receiving A
A composition as defined in Table 12

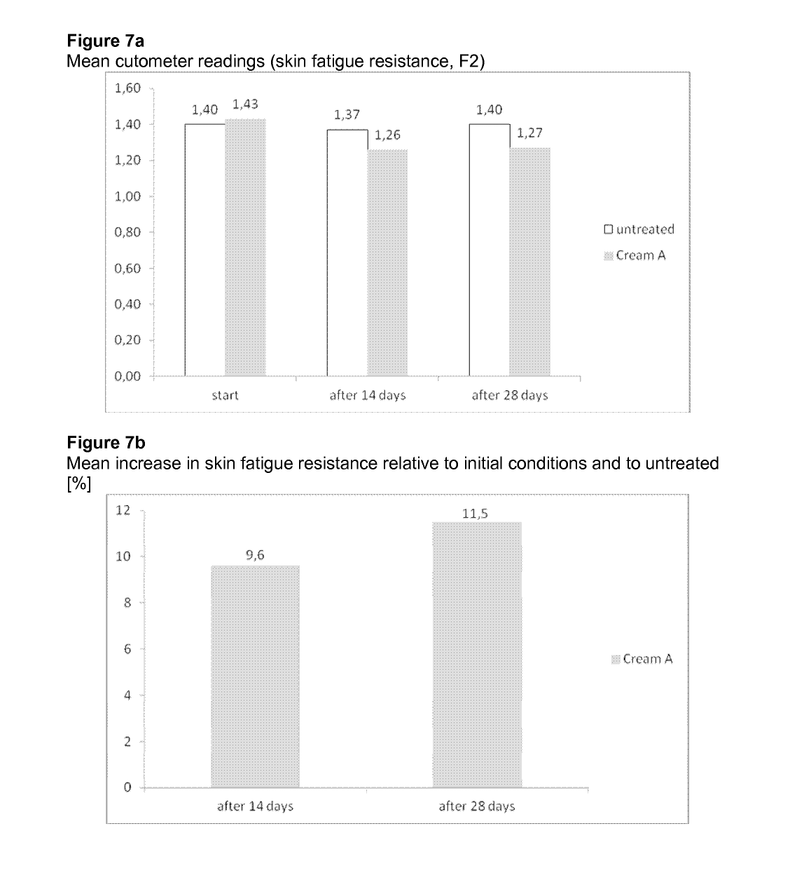

In assessing skin fatigue resistance, evaluated are the changes in the parameter F2 in the treated area in comparison to the changes in the untreated area. The absolute changes by area and time point are shown FIG. 7a. A decrease in F2 corresponds to an increase in fatigue resistance.

After both 14 and 28 days of treatment, a statistically significant (p<0.05) decrease in F2 was observed in the product treated test area as compared to the changes in the untreated area. The test product was found to statistically significantly increase skin fatigue resistance; after 28 days of treatment while positive effect could be detected in 80% of the study participants. The respective percentage changes as compared to the initial condition and with regard of the changes in the untreated area are shown in FIG. 7b.

### CONCLUSION

The composition according to the present invention was found to statistically significantly enhance the biomechanical properties of the skin towards the firm-elastic optimum. After 28 days of treatment, a mean increase by 17% (firmness), 10% (elasticity) and 12% (fatigue resistance) was observed and a positive effect of the test product was detected in 95% (firmness), 85% (elasticity) and 80% (fatigue resistance) of the volunteers.

### Example 2b

### Evaluation of the Efficacy in View of Wrinkle Depth, Area/Volume

TABLE 20
Description of the evaluation

| | |
|---|---|
| Subjects | Number: 20 (+1 reserve subject) |
| | Age: 36-65 years (average: 48.2) |
| | Sex: female |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Product tested | Composition as defined in Table 9 |
| Region tested | inner side of forearms |
| | face (Crow's feet/temple) |
| Application | Duration: 28 days |
| | Frequency: twice daily |
| Evaluation method | Descriptive statistics (average, median, minimum, |
| | maximum, variance, standard error, standard deviation); |
| | Wilcoxon Rank Test |
| Design of study | Day 0: |
| | Determination of the parameters in the test areas |
| | First test product application |
| | Day 14: |
| | Determination of the parameters 8-12 hours |
| | following the last daily test product application |
| | Day 28: |
| | Determination of the parameters 8-12 hours |
| | following the last daily test product application |
| | The subjects used the test products (approximately |
| | 2 mg/cm²) twice daily in the morning and in the evening. |
| Test parameters | Determination of wrinkle depth by means of PRIMOS ® 5.7 |
| | high-res (GFMeβtechnik GmbH, Teltow, Germany) |
| Measurement | The crow feet area is recorded as a 3D topography using |
| | the PRIMOS system. |
| | PRIMOS compact high-res S/N 108-00041, Software |
| | Version 5.7. |
| Expression of | Wrinkle depth was assessed by means of the parameter |
| results | Rₘₐₓ that is defined as the maximum vertical distance |
| Wrinkle depth | from the highest peak to the lowest valley of five |
| (Rₘₐₓ, μm) | segments of equal length. To mitigate locational effects, |
| | the evaluation was conducted using the arithmetic |
| | average of Rₘₐₓ from 50 parallel cuts, drawn |
| | perpendicular to the main wrinkle through the entire |
| | measurement (0.5 mm borders). |
| Mean wrinkle | Mean wrinkle depth as calculated by the wrinkle |
| depth (μm) | analysis module (detection artefacts manually excluded) |
| Total wrinkle area | Total measurement area covered by wrinkles detected |
| (mm²) | by the wrinkle analysis module (detection artefacts |
| | manually excluded). |
| Total wrinkle | Total volume of the wrinkles detected by the wrinkle |
| volume (mm³) | analysis module (detection artefacts manually |
| | excluded). Can be calculated from mean depth and |
| | covered area. |

TABLE 21
PRIMOS readings for wrinkle depth, area/volume
(PRIMOS readings (vers. 5.7))

| | | | |
|---|---|---|---|
| Depth | | | |
| (Rₘₐₓ, μm) | | | |
| (Rₘₐₓ, μm) | Mean | Area | Volume |

Start:

| | | | | |
|---|---|---|---|---|
| 1 | 483.56 | 185.00 | 22.44 | 4.15 |
| 2 | 415.54 | 227.00 | 29.95 | 6.80 |
| 3 | 360.24 | 185.00 | 23.27 | 4.30 |
| 4 | 398.06 | 158.00 | 17.48 | 2.76 |
| 5 | 333.80 | 161.00 | 24.22 | 3.90 |
| 6 | 448.08 | 230.00 | 30.44 | 7.00 |
| 7 | 339.50 | 165.00 | 29.91 | 4.94 |
| 8 | 448.28 | 226.00 | 35.15 | 7.94 |
| 9 | 298.56 | 100.00 | 10.13 | 1.01 |
| 10 | 331.56 | 144.00 | 31.73 | 4.57 |
| 11 | 359.02 | 133.00 | 23.80 | 3.17 |
| 12 | 276.76 | 125.00 | 36.56 | 4.57 |
| 13 | 437.88 | 177.00 | 29.58 | 5.24 |
| 14 | 288.24 | 166.00 | 13.52 | 2.24 |
| 15 | 345.36 | 113.00 | 24.70 | 2.79 |
| 16 | 348.78 | 146.00 | 26.89 | 3.93 |
| 17 | 461.96 | 172.00 | 21.78 | 3.75 |
| 18 | 345.36 | 114.00 | 18.25 | 2.08 |
| 19 | 526.68 | 307.00 | 37.41 | 11.48 |
| 20 | 474.90 | 199.00 | 18.44 | 3.67 |
| Average | 386.11 | 171.65 | 25.28 | 4.51 |
| S.D. | 71.63 | 49.33 | 7.46 | 2.36 |
| Median | 359.63 | 165.50 | 24.46 | 4.04 |

After 14 days:

| | | | | |
|---|---|---|---|---|
| 1 | 461.82 | 201.00 | 20.53 | 4.13 |
| 2 | 325.48 | 184.00 | 28.49 | 5.24 |
| 3 | 308.10 | 147.00 | 19.23 | 2.83 |
| 4 | 331.48 | 141.00 | 18.50 | 2.61 |
| 5 | 305.86 | 138.00 | 21.34 | 2.94 |
| 6 | 385.14 | 218.00 | 28.60 | 6.23 |
| 7 | 344.82 | 167.00 | 29.17 | 4.87 |
| 8 | 402.80 | 195.00 | 33.84 | 6.60 |
| 9 | 313.54 | 89.00 | 11.87 | 1.06 |
| 10 | 334.66 | 132.00 | 31.32 | 4.13 |
| 11 | 354.72 | 133.00 | 24.07 | 3.20 |
| 12 | 266.40 | 98.00 | 36.59 | 3.59 |
| 13 | 385.46 | 154.00 | 28.01 | 4.31 |
| 14 | 268.62 | 159.00 | 12.95 | 2.06 |
| 15 | 364.76 | 116.00 | 22.80 | 2.64 |
| 16 | 277.40 | 110.00 | 25.42 | 2.80 |
| 17 | 375.90 | 153.00 | 20.15 | 3.08 |
| 18 | 314.68 | 103.00 | 18.91 | 1.95 |
| 19 | 461.46 | 294.00 | 34.82 | 10.24 |
| 20 | 451.34 | 206.00 | 18.79 | 3.87 |
| Average | 351.72 | 156.90 | 24.27 | 3.92 |
| S.D. | 59.69 | 49.18 | 6.96 | 2.04 |
| Median | 339.74 | 150.00 | 23.44 | 3.39 |

After 28 days:

| | | | | |
|---|---|---|---|---|
| 1 | 418.24 | 191.00 | 17.68 | 3.38 |
| 2 | 337.80 | 198.00 | 29.75 | 5.89 |
| 3 | 250.24 | 143.00 | 17.77 | 2.54 |
| 4 | 299.86 | 133.00 | 13.27 | 1.76 |
| 5 | 290.76 | 144.00 | 18.78 | 2.70 |
| 6 | 359.76 | 211.00 | 27.15 | 5.73 |
| 7 | 313.84 | 147.00 | 28.38 | 4.17 |
| 8 | 358.18 | 195.00 | 31.59 | 6.16 |
| 9 | 276.78 | 94.00 | 10.71 | 1.01 |
| 10 | 242.64 | 126.00 | 29.75 | 3.75 |
| 11 | 287.50 | 112.00 | 19.40 | 2.17 |
| 12 | 250.36 | 101.00 | 34.86 | 3.52 |
| 13 | 368.08 | 140.00 | 25.54 | 3.58 |
| 14 | 255.26 | 154.00 | 9.77 | 1.50 |
| 15 | 344.18 | 94.00 | 18.54 | 1.74 |
| 16 | 243.28 | 105.00 | 24.78 | 2.60 |
| 17 | 346.70 | 135.00 | 18.55 | 2.50 |
| 18 | 311.52 | 100.00 | 15.77 | 1.58 |
| 19 | 386.68 | 251.00 | 33.44 | 8.39 |
| 20 | 375.70 | 192.00 | 16.24 | 3.12 |
| Average | 315.87 | 148.30 | 22.09 | 3.39 |
| S.D. | 53.26 | 44.47 | 7.60 | 1.88 |
| Median | 312.68 | 141.50 | 19.09 | 2.91 |

TABLE 22
Decrease in wrinkle depth, area/volume relative to initial
conditions (corrected PRIMOS readings (vers. 5.7)) [%]

| | | | |
|---|---|---|---|
| Depth | Mean | Area | Volume |

After 14 days

| | | | | |
|---|---|---|---|---|
| 1 | −4.5 | 8.6 | −8.5 | −0.6 |
| 2 | −21.7 | −18.9 | −4.9 | −22.9 |
| 3 | −14.5 | −20.5 | −17.4 | −34.3 |
| 4 | −16.7 | −10.8 | 5.8 | −5.6 |
| 5 | −8.4 | −14.3 | −11.9 | −24.5 |
| 6 | −14.0 | −5.2 | −6.0 | −10.9 |
| 7 | 1.6 | 1.2 | −2.5 | −1.3 |
| 8 | −10.1 | −13.7 | −3.7 | −16.9 |
| 9 | 5.0 | −11.0 | 17.2 | 4.3 |
| 10 | 0.9 | −8.3 | −1.3 | −9.5 |
| 11 | −1.2 | 0.0 | 1.1 | 1.1 |
| 12 | −3.7 | −21.6 | 0.1 | −21.5 |
| 13 | −12.0 | −13.0 | −5.3 | −17.6 |
| 14 | −6.8 | −4.2 | −4.2 | −8.3 |
| 15 | 5.6 | 2.7 | −7.7 | −5.2 |
| 16 | −20.5 | −24.7 | −5.5 | −28.8 |
| 17 | −18.6 | −11.0 | −7.5 | −17.7 |
| 18 | −8.9 | −9.6 | 3.6 | −6.4 |
| 19 | −12.4 | −4.2 | −6.9 | −10.9 |
| 20 | −5.0 | 3.5 | 1.9 | 5.5 |
| Average | −8.3 | −8.8 | −3.2 | −11.6 |
| S.D. | 8.2 | 9.1 | 7.2 | 11.1 |
| Median | −8.6 | −10.2 | −4.5 | −10.2 |
| Impr.* | 80 | 75 | 70 | 85 |

After 28 days

| | | | | |
|---|---|---|---|---|
| 1 | −13.5 | 3.2 | −21.2 | −18.7 |
| 2 | −18.7 | −12.8 | −0.7 | −13.4 |
| 3 | −30.5 | −22.7 | −23.6 | −41.0 |
| 4 | −24.7 | −15.8 | −24.1 | −36.1 |
| 5 | −12.9 | −10.6 | −22.5 | −30.6 |
| 6 | −19.7 | −8.3 | −10.8 | −18.2 |
| 7 | −7.6 | −10.9 | −5.1 | −15.5 |
| 8 | −20.1 | −13.7 | −10.1 | −22.5 |
| 9 | −7.3 | −6.0 | 5.7 | −0.6 |
| 10 | −26.8 | −12.5 | −6.2 | −18.0 |
| 11 | −19.9 | −15.8 | −18.5 | −31.4 |
| 12 | −9.5 | −19.2 | −4.6 | −23.0 |
| 13 | −15.9 | −20.9 | −13.7 | −31.7 |
| 14 | −11.4 | −7.2 | −27.7 | −33.0 |
| 15 | −0.3 | −16.8 | −24.9 | −37.6 |
| 16 | −30.2 | −28.1 | −7.8 | −33.7 |
| 17 | −25.0 | −21.5 | −14.8 | −33.2 |
| 18 | −9.8 | −12.3 | −13.6 | −24.2 |
| 19 | −26.6 | −18.2 | −10.6 | −26.9 |
| 20 | −20.9 | −3.5 | −11.9 | −15.0 |
| Average | −17.6 | −13.7 | −13.3 | −25.2 |
| S.D. | 8.4 | 7.3 | 8.9 | 10.1 |
| Median | −19.2 | −13.2 | −12.8 | −25.6 |
| Impr.* | 100 | 95 | 95 | 100 |

*% of subjects with relative improvement in test area as compared to initial condition and corrected by changes in untreated area

Evaluated are the four wrinkle parameters—depth (classic cut analysis & wrinkle analysis module), volume (wrinkle analysis module) & area parameters (wrinkle analysis module)—in comparison to the initial condition in the same respective test area.

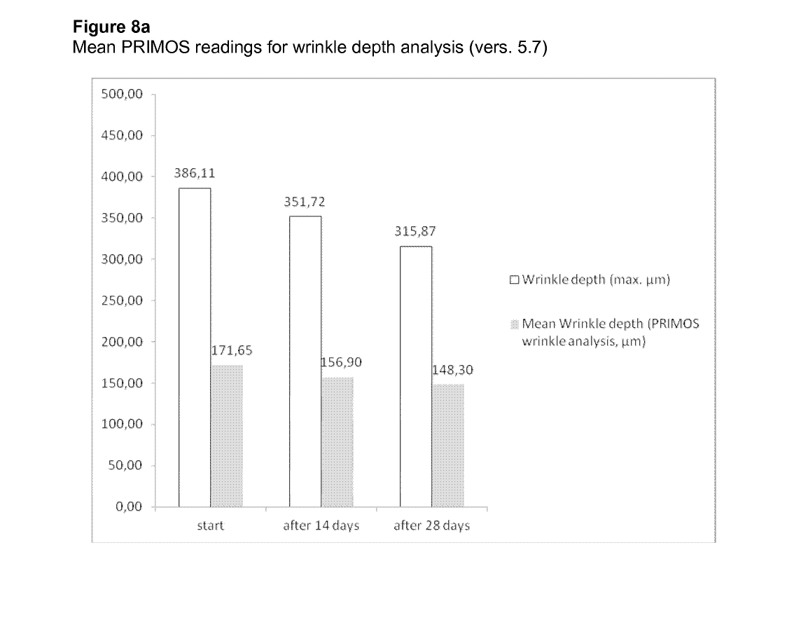

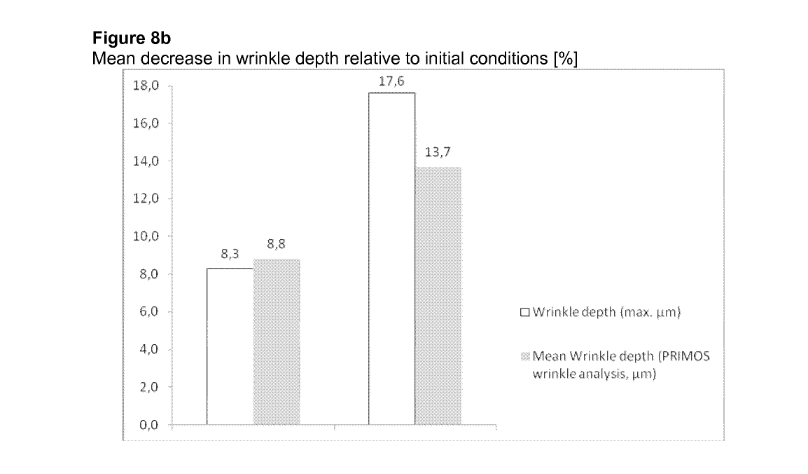

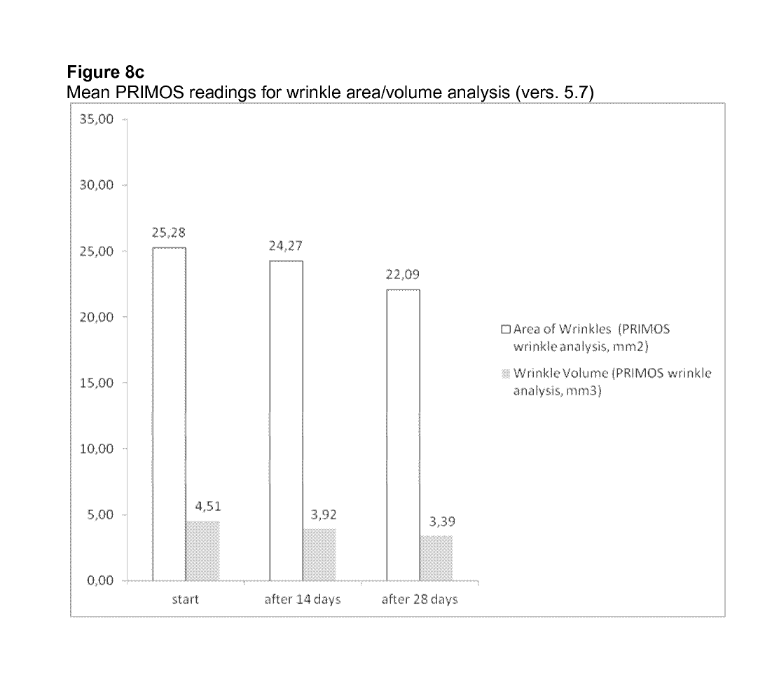

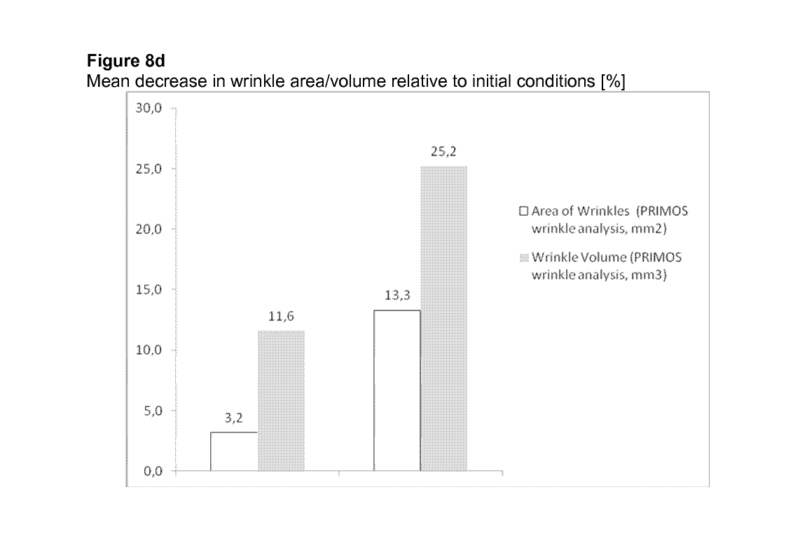

A decrease in all respective measurement values corresponds to a decrease in wrinkle depth/area/volume (FIG. 8a—wrinkle depth and FIG. 8c—wrinkle area/volume).

After both 14 and 28 days of treatment, a statistically significant (p<0.05) decrease was detected in all four measured parameters depth (classical cut analysis & wrinkle analysis module), area and volume for the test product as compared to the respective initial condition. The composition according to the present invention was found to statistically significantly decrease wrinkle depth, area and volume in regular treatment; after 28 days of treatment a positive effect could be detected in 100% (Depth—classical cut analysis; Volume)/95% (Depth—wrinkle analysis module/Area). The respective percentage changes as compared to the initial condition are shown in FIGS. 8b and 8d.

### Example 2c

### Evaluation of the Efficacy Against Photooxidative Stress in Skin Induced by UVA-Irradiation

TABLE 23
Description of the evaluation

| | |
|---|---|
| Subjects | Number: 20 |
| | Age: 31-61 years (average: 45.2) |
| | Sex: female |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Product tested | Composition as defined in Table 9 |
| Region tested | inner side of forearms |
| Application | Single application |
| | On the inner side of the forearm of each subject |
| | symmetrically opposed areas were defined. The |
| | formulation was applied at a dose of approximately |
| | 2 mg/cm². 30 minutes after the application the β- |
| | carotene solution (5 mg/100 ml) in n-hexane (80 μl) was |
| | applied on each test site. 5 minutes later, the coloration |
| | was measured by Chromameter (b* blue-yellow axis) and |
| | subsequently the test sites were irradiated (UVA: 10 |
| | Joule/cm²). One area (Control) remained untreated, but was |
| | colorized and irradiated. Five minutes after irradiation, |
| | coloration was measured again. |
| Evaluation | Change of color of β-carotene |
| method | Descriptive statistics: average, median, minimum, |
| | maximum, standard error, standard deviation; Wilcoxon |
| | Rank Test |
| Time of | Before and after UVA irradiation |
| evaluation | |
| Test method | β-carotene is a natural substance having orange-yellow |
| | color. Under the influence of UVA light it discolors as a |
| | result of a chemical process involving reactive oxygen |
| | species like singlet oxygen, the superoxide radical, |
| | hydrogen peroxide and the hydroxyl radical. |
| | If a product applied to the skin has antioxidant |
| | properties the color change in β-carotene is attenuated |
| | or inhibited. |
| Expression of | The results are expressed as the rate of inhibition |
| results | relative to the untreated area (Color index): |
| | $100 - \frac{{Sample}_{colorized} - {Sample}_{irradiated}}{{Control}_{colorized} - {Control}_{irradiated}} \times 100$ |
| | Colorized means color reading after coloration with (β- |
| | carotene |
| | Irradiated means color reading after irradiation |
| | The color index in % corresponds directly to the efficacy |
| | against free radicals. |
| Measurement of | Skin color is measured by Minolta Chromameter CR 400 |
| discoloration | (Minolta, Japan) using the Commission International de |
| | l'eclairage (CIE) L*a*b* color space—a standardized, |
| | device independent system to express Colors adjusted |
| | to the non-linear Color sensitivity of human eye. In the |
| | L*a*b* color space, a color is expressed in a three- |
| | dimensional coordinate system with green-red (a*—negative |
| | values are green, positive values are red), |
| | blue-yellow (b*—negative values are blue, positive |
| | values are yellow) and L* axes (lightness). The a* axis |
| | ranges from −150 to +100, the b* axis from −100 to +150 |
| | and the L* axis from 0 (black) to 100 (white). During |
| | measurement, the skin surface is illuminated by a |
| | Xenon flashlight and remitted light registered and |
| | analysed by a photoreceiver. The Chromameter CR 400 |
| | is sensitive and accurate for the characterisation of skin |
| | color and measures a spot of 8 mm diameter. Before |
| | each measuring series, the instrument was calibrated |
| | against a standard white tile. Measurements were done |
| | according to the guidelines of the European Society of |
| | Contact Dermatitis (Fullerton et al., Guidelines for |
| | measurement of skin colour and erythema. Contact |
| | Dermatitis 1996: 35; 1-10). Chromameter used for |
| | this study: S/N C8202118. |
| Biometry | Measurement data is automatically computerised and |
| | after validity check and quality assurance stored |
| | centrally in a database. Evaluation is conducted using |
| | the software NAG ® Statistical Add-Ins for Excel-NAG |
| | Ltd., United Kingdom. The data were analyzed by |
| | Wilcoxon Rank Test. The 0.05 level was selected as the |
| | point of minimal acceptance of statistical significance. |

TABLE 24
Chromameter readings (b*) for discoloration of carotene
after UV t1-t0

| | untr. | A |
|---|---|---|
| 1 | −19.79 | −4.43 |
| 2 | −15.80 | −0.87 |
| 3 | −19.61 | −6.34 |
| 4 | −20.02 | −12.05 |
| 5 | −12.51 | −0.54 |
| 6 | −16.53 | −3.39 |
| 7 | −18.23 | −4.12 |
| 8 | −19.71 | −4.71 |
| 9 | −20.65 | 0.51 |
| 10 | −20.02 | −6.09 |
| 11 | −23.46 | −5.53 |
| 12 | −19.75 | −7.56 |
| 13 | −22.82 | −4.45 |
| 14 | −22.89 | −8.77 |
| 15 | −17.97 | −6.01 |
| 16 | −17.06 | −5.44 |
| 17 | −24.53 | −6.34 |
| 18 | −12.97 | −5.37 |
| 19 | −17.03 | −2.70 |
| 20 | −16.69 | −5.69 |
| Average | −18.90 | −4.99 |
| S.D. | 3.21 | 2.85 |
| Median | −19.66 | −5.41 |

untr. subjects not receiving A
A composition as defined in Table 12

TABLE 25
Color index [%]

| | A |
|---|---|
| 1 | 77.6 |
| 2 | 94.5 |
| 3 | 67.7 |
| 4 | 39.8 |
| 5 | 95.7 |
| 6 | 79.5 |
| 7 | 77.4 |
| 8 | 76.1 |
| 9 | 102.5 |
| 10 | 69.6 |
| 11 | 76.4 |
| 12 | 61.7 |
| 13 | 80.5 |
| 14 | 61.7 |
| 15 | 66.6 |
| 16 | 68.1 |
| 17 | 74.2 |
| 18 | 58.6 |
| 19 | 84.1 |
| 20 | 65.9 |
| Average | 73.9 |
| S.D. | 14.2 |
| Median | 75.1 |

A composition as defined in Table 12

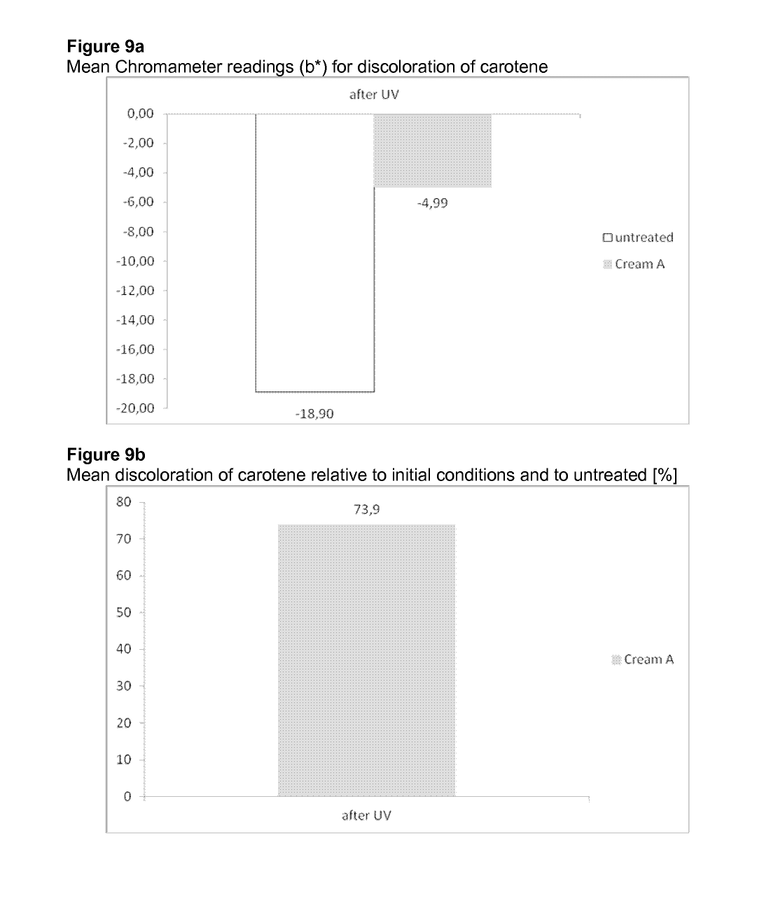

Evaluated are the changes in the b* values in the treated area in comparison to the changes in the untreated area. A decrease in the measurement values corresponds to a decrease in yellow skin coloration. The changes from the UV irradiation are shown in FIG. 9a.

The composition according to the present invention prevented UVA-induced discoloration of β-carotene (p<0.05 versus untreated). The respective changes in discoloration in the treated area as percentages relative to the initial condition and to the colour changes in the untreated (Color Index) area are reported in FIG. 9b.

### Example 2d

### Determination of Skin Thickness

TABLE 26
Description of the evaluation

| | |
|---|---|
| Subjects | Number: 20 (+1 reserve subject) |
| | Age: 36-65 years (average: 48.2) |
| | Sex: female |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Product tested | Composition as defined in Table 9 |
| Region tested | inner side of forearms |
| | face (Crow's feet/temple) |
| Application | Duration: 28 days |
| | Frequency: twice daily |
| Evaluation method | Descriptive statistics (average, median, minimum, |
| | maximum, variance, standard error, standard deviation); |
| | Wilcoxon Rank Test |
| Design of study | Day 0: |
| | Determination of the skin thickness in the test |
| | areas |
| | First test product application |
| | Day 14: |
| | Determination of the skin thickness 8-12 hours |
| | following the last daily test product application |
| | Day 28: |
| | Determination of the skin thickness 8-12 hours |
| | following the last daily test product application |
| | The subjects used the test products (approximately |
| | 2 mg/cm²) twice daily in the morning and in the evening. |
| Test parameters | Determination of skin thickness using |
| | COLLAGENOSON ®“ICU” (Minhorst GmbH & Co., Meudt, |
| | Germany) |
| Measurement | Examination of the skin up to a depth of approximately 6 mm |
| Expression of | Evaluated are the changes in skin thickness measured by |
| results | ultrasound on the temple (centered between eye and |
| | hairline) in comparison to the initial condition in the same |
| | test area. |

TABLE 27
Collagenoson ICU readings for skin thickness (Collagenoson
ICU readings (temple, mm), delta values)

| | 14 days | 28 days |
|---|---|---|
| | A | A |
| 1 | 0.05 | 0.09 |
| 2 | 0.34 | 0.32 |
| 3 | 0.18 | 0.17 |
| 4 | 0.04 | 0.17 |
| 5 | −0.11 | −0.02 |
| 6 | 0.06 | 0.13 |
| 7 | 0.05 | 0.19 |
| 8 | −0.01 | −0.01 |
| 9 | 0.05 | 0.14 |
| 10 | 0.05 | 0.07 |
| 11 | 0.11 | 0.07 |
| 12 | −0.15 | −0.09 |
| 13 | 0.17 | 0.11 |
| 14 | 0.09 | 0.14 |
| 15 | 0.16 | 0.07 |
| 16 | −0.04 | −0.11 |
| 17 | 0.14 | 0.19 |
| 18 | −0.14 | −0.05 |
| 19 | 0.04 | 0.07 |
| 20 | 0.01 | 0.17 |
| Average | 0.05 | 0.09 |
| S.D. | 0.12 | 0.11 |
| Median | 0.05 | 0.10 |
| Impr.* | 75 | 75 |

A composition as defined in Table 12
*% of subjects with relative improvement in test area as compared to initial condition and corrected by changes in untreated area

After both, 14 and 28 days of treatment, a statistically significant (p<0.05) increase in skin thickness could be observed (FIG. 11).

### Example 2e

### Determination of Transepidermal Waterloss (TEWL) and Skin Redness

TABLE 28
Description of the evaluation

| | |
|---|---|
| Subjects | Number: 20 |
| | Age: 26-60 years (average: 42.4) |
| | Sex: 14 female, 6 male |
| | Clinically healthy subjects |
| | The subjects were instructed not to use any topical |
| | preparations on the test areas starting from seven days |
| | prior to testing and until the end of the test. |
| | No incompatibility was observed in any of the subjects. |
| Product tested | Composition as defined in Table 9 |
| Region tested | inner side of forearms |
| Application | duration: 14 days (7 for regenerative & 7 for protective |
| | efficacy) |
| | frequency: twice daily |
| Evaluation method | Change of color of β-carotene |
| | Descriptive statistics: average, median, minimum, |
| | maximum, standard error, standard deviation; Wilcoxon |
| | Rank Test |
| Time of evaluation | initial condition (start), 6 hours after last washing for |
| | induction of SDS irritation (after SDS), after 1, 2, 4 and 7 |
| | days of product application, after 7 days of concurrent |
| | product application & SDS washing (after 1 week SDS) |
| Test method redness | With an eye to the task at hand, the measurements |
| | were taken exclusively in the L*a*b* colorimetric system |
| | (also designated as CIELAB system). L* represents the |
| | brightness, while a* and b* the hue and color saturation. |
| | a* shows the position on the red-green axis and b* on |
| | the yellow-blue axis. An increase in skin reddening is |
| | shown by an increase in the a* value. The values are |
| | internationally defined absolute values. Measurements |
| | were conducted according to the guidelines of the |
| | European Society of Contact Dermatitis (Fullerton et al., |
| | Guidelines for measurement of skin colour and |
| | erythema. Contact Dermatitis 1996: 35; 1-10). Each |
| | value is the average of three recordings. Chromameter |
| | used in this study: SN C 8202118. |
| TEWL | Measurements of TEWL were performed with the |
| | Tewameter TM 210 (Courage & Khazaka, Cologne, |
| | Germany). The Tewameter is a device for measurement |
| | of water evaporation on skin surfaces based on the |
| | diffusion principle discovered by A. Fick in 1885. The |
| | TEWL is calculated automatically and expressed digitally |
| | in g/m²h. Measurements were carried out in accordance |
| | with the guidelines of the standardisation group of the |
| | European Contact Dermatitis Society (Pinnagoda et al. |
| | Contact Dermatitis 1990: 22; 164-178). Each value is the |
| | average of three recordings. Tewameter used in this |
| | study: SN 27 022 074. |
| Performance of test | To study regenerative properties skin irritation was |
| Regenerative properties | induced in the test areas by 1 week of twice daily |
| | washing of the forearms with Sodium Dodecyl Sulphate |
| | (SDS, purity: 99%) 5% in distilled water. |
| | 6 hours after the last SDS washing, skin redness and |
| | TEWL were recorded (after SDS). After the |
| | measurements the test product was applied |
| | (randomized location) approximately 2 mg/cm²: one area |
| | was left untreated and served as control. Twice daily |
| | application took place (in the morning and evening) and |
| | measurements were taken after 1, 2, 4 and 7 days of |
| | treatment 6 hours after the respective last product |
| | application to study the effect of the product treatment |
| | on the recovery process of the skin. |
| Protective properties | To study the protective properties of the test product, |
| | the subjects were asked to continue the application of |
| | the test product for another 7 days, but in addition to |
| | also resume the twice daily washing routine with the 5% |
| | SDS solution (in both the product & untreated test |
| | areas) with a 1 hour delay after the respective test |
| | product application. A final measurement was taken after |
| | the 7 days 6 hours after the final SDS washing to study |
| | the protective effect of the product treatment. |
| Biometry | Evaluation is conducted using the software NAG ® |
| | Statistical Add-Ins for Excel - NAG Ltd., United |
| | Kingdom. The data were analyzed by Wilcoxon Rank |
| | Test. The 0.05 level was selected as the point of minimal |
| | acceptance of statistical significance. |
| | For assessment of product efficacy, evaluated are the |
| | changes to the respective baseline values (after SDS to |
| | start; days 1, 2, 4, 7 to after SDS; after 7 days of SDS to |
| | end of regeneration) in the treated area as compared to |
| | the changes in the untreated area. To assess the overall |
| | effect of the SDS treatment within a test area, evaluated |
| | are the changes to the respective initial condition in the |
| | same test area. |

TABLE 29
Chromameter readings (a*) for skin redness, 1 day, 2 days, 4 days, 7 days
after SDS - regeneration (delta Chromameter readings)

| | after SDS | | after 1 day | | after 2 days | | after 4 days | | after 7 days | |
|---|---|---|---|---|---|---|---|---|---|---|
| | t1-t0 | | t2-t1 | | t3-t1 | | t4-t1 | | t5-t1 | |
| | untr | A | untr | A | untr | A | untr | A | untr | A |
| 1 | 2.37 | 2.49 | −0.12 | −0.31 | 0.34 | −0.35 | −1.23 | −1.15 | −2.43 | −1.62 |
| 2 | 1.83 | 1.19 | 0.28 | 0.35 | −1.30 | −0.99 | −1.79 | −0.73 | −1.30 | −0.82 |
| 3 | 1.11 | 1.52 | −0.62 | −0.61 | −0.15 | −0.28 | −1.03 | −0.24 | 1.08 | −0.32 |
| 4 | 4.14 | 3.10 | −0.73 | −0.96 | −0.81 | −1.18 | −3.59 | −2.11 | −3.26 | −2.96 |
| 5 | 1.29 | 2.49 | −1.42 | −2.27 | −1.18 | −3.86 | −0.89 | −3.92 | −1.09 | −3.63 |
| 6 | 3.03 | 3.62 | 1.77 | −0.02 | −0.41 | −2.38 | −2.10 | −1.45 | −3.11 | −2.70 |
| 7 | 0.82 | 0.15 | 0.58 | −0.66 | −1.28 | −0.64 | −0.39 | −1.20 | −0.84 | 0.57 |
| 8 | 1.42 | 2.51 | 0.58 | −1.57 | −0.65 | −1.68 | −0.38 | −1.27 | −1.28 | −1.09 |
| 9 | 2.65 | 3.04 | −0.49 | −0.04 | −0.33 | −0.35 | −3.08 | −0.31 | −3.11 | −2.21 |
| 10 | 0.90 | 1.06 | 0.23 | −0.64 | −0.69 | −0.87 | −0.17 | −0.59 | −0.73 | −1.80 |
| 11 | 0.62 | 0.44 | −0.57 | −0.61 | −0.01 | −1.05 | −0.05 | −1.69 | −1.48 | −1.30 |
| 12 | 0.17 | 1.61 | 0.55 | −0.42 | 0.13 | −0.80 | 0.21 | −1.46 | 0.28 | −1.35 |
| 13 | −0.06 | 0.92 | 1.24 | 0.34 | 0.41 | −1.68 | −0.41 | −1.43 | 0.05 | −1.95 |
| 14 | 1.66 | 0.19 | 0.07 | 1.25 | −0.19 | −0.74 | −1.07 | −0.59 | −1.43 | −1.22 |
| 15 | 2.24 | 1.14 | 0.17 | −1.19 | −1.05 | −1.55 | −1.40 | −1.56 | −2.57 | −1.79 |
| 16 | 0.87 | 1.33 | 0.48 | −1.00 | −0.22 | −0.74 | 0.13 | −1.97 | −0.29 | −0.78 |
| 17 | 1.55 | 1.50 | −0.72 | −1.21 | −0.65 | −0.74 | −1.85 | −1.34 | −2.85 | −2.33 |
| 18 | 2.00 | 1.03 | −0.16 | −0.72 | −1.29 | −0.38 | −1.71 | −0.85 | −1.78 | −0.89 |
| 19 | −0.08 | −0.20 | −0.48 | 0.15 | −0.37 | 0.16 | −0.06 | 0.18 | −0.06 | 0.14 |
| 20 | 1.99 | 0.05 | −1.00 | −0.34 | −0.78 | −0.04 | −1.00 | −1.37 | −1.24 | −0.11 |
| Average | 1.53 | 1.46 | −0.02 | −0.52 | −0.52 | −1.01 | −1.09 | −1.25 | −1.37 | −1.41 |
| S.D. | 1.06 | 1.10 | 0.77 | 0.77 | 0.53 | 0.91 | 1.04 | 0.86 | 1.23 | 1.06 |
| Median | 1.49 | 1.26 | −0.03 | −0.61 | −0.53 | −0.77 | −1.01 | −1.31 | −1.29 | −1.33 |

untr. subjects not receiving A
A composition as defined in Table 12

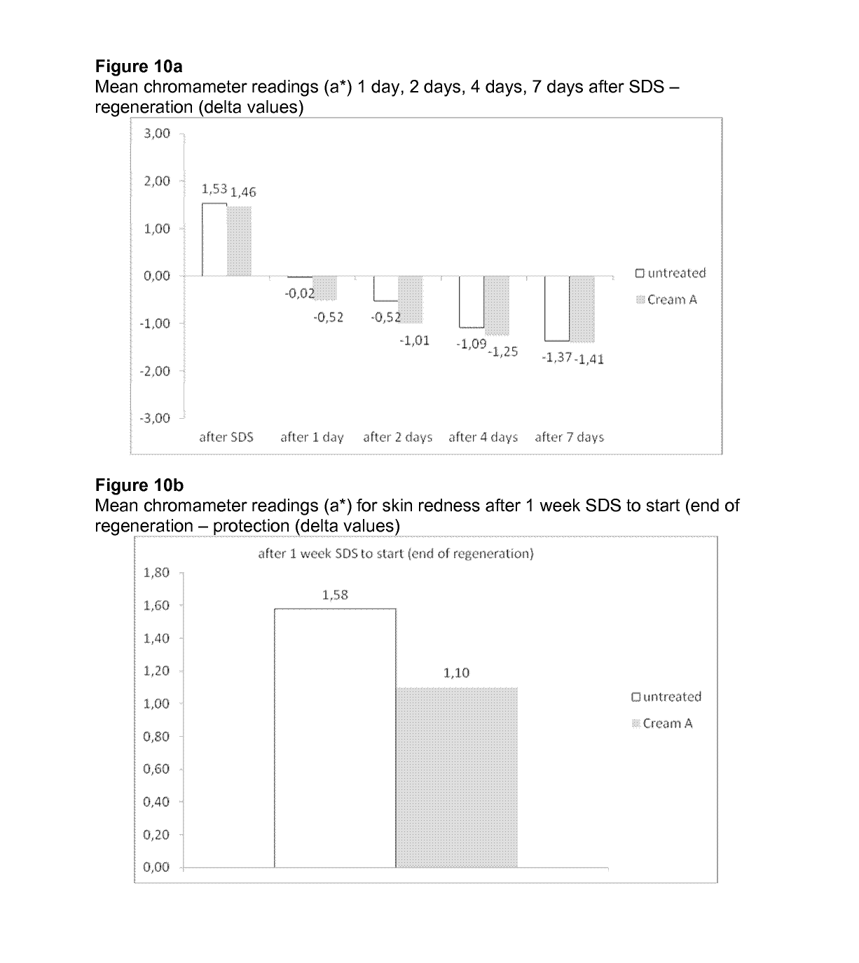

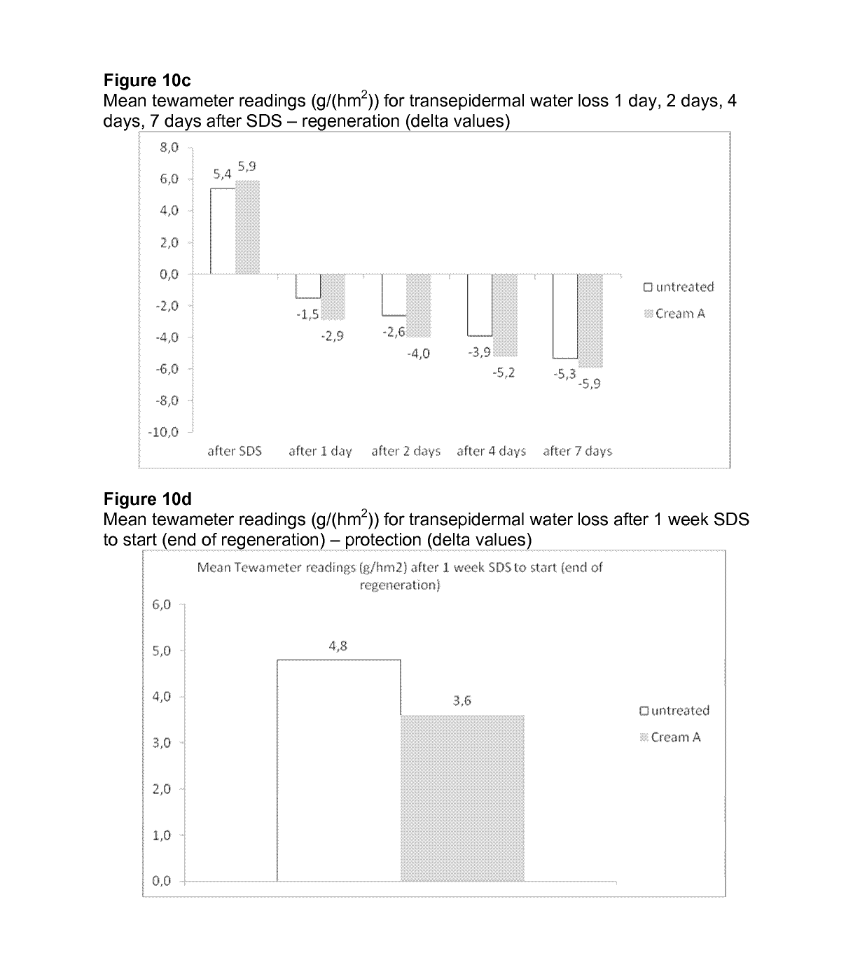

The solution of 5% SDS in water induced in most of the subjects after 1 week of washing measurable and overall statistically significant skin reddening in both test areas. During the following application phase, skin redness (a*) was found to decrease both in the untreated and product treated test areas. The decrease in the product treated test area was found to be statistically significant larger after both 1 and 2 days of treatment as compared the changes in the untreated control area with “after SDS” taken as baseline (p<0.05) (FIG. 10a). On days 2 (area treated with test product) and 7 (untreated control area), skin redness was found (in statistical terms, p<0.05) to have returned to its original level as compared to the respective initial condition.

TABLE 30
Chromameter readings (a*) for skin redness after 1 week
SDS to start (end of regeneration) - protection (delta
Chromameter readings (a*)

after 1 week
SDS t1-t0

| | untr. | A |
|---|---|---|
| 1 | 1.70 | 1.15 |
| 2 | 1.29 | 0.55 |
| 3 | 2.40 | 2.67 |
| 4 | 4.70 | 3.49 |
| 5 | 2.78 | 1.20 |
| 6 | 4.23 | 2.63 |
| 7 | −0.11 | 0.63 |
| 8 | 2.85 | 3.06 |
| 9 | 1.69 | 0.30 |
| 10 | 1.14 | 0.55 |
| 11 | −0.12 | 0.41 |
| 12 | 1.31 | 0.49 |
| 13 | 1.29 | 0.26 |
| 14 | 0.63 | −0.77 |
| 15 | 0.81 | 1.56 |
| 16 | 0.45 | −0.20 |
| 17 | 1.66 | 1.15 |
| 18 | 1.91 | 0.74 |
| 19 | −1.03 | 0.51 |
| 20 | 2.04 | 1.58 |
| Average | 1.58 | 1.10 |
| S.D. | 1.39 | 1.11 |
| Median | 1.49 | 0.68 |

untr. subjects not receiving A
A composition as defined in Table 12

7 days after the discontinuation of SDS washing, it was resumed again in addition to the product application (SDS washing 1 hour after the respective product application) and a repeated statistically significant increase in skin redness (a*) was observed both in the untreated and product treated test areas (FIG. 10b). The increase in the product treated test area was found to be statistically significantly (p<0.05) smaller than the increase in the untreated, SDS washed, control area.

The composition according to the present invention has both a statistically significant regeneration effect and protection effect on skin redness as induced by the damaging properties of the surfactant SDS (i.e. to accelerate recovery from induced skin redness and to protect from the formation of induced skin redness).

TABLE 31
Tewameter readings for transepidermal water loss 1 day, 2 days, 4 days, 7
days after SDS - regeneration (delta Tewameter readings (g/hm²))

| | after SDS | | after 1 day | | after 2 days | | after 4 days | | after 7 days | |
|---|---|---|---|---|---|---|---|---|---|---|
| | t1-t0 | | t2-t1 | | t3-t1 | | t4-t1 | | t5-t1 | |
| | untr. | A | untr. | A | untr. | A | untr. | A | untr. | A |
| 1 | 7.6 | 5.2 | −1.7 | −2.8 | −4.8 | −4.7 | −5.8 | −5.2 | −8.2 | −5.5 |
| 2 | 7.2 | 6.6 | −0.4 | −3.5 | −3.3 | −6.6 | −5.0 | −7.8 | −6.8 | −7.4 |
| 3 | 3.0 | 6.0 | −0.4 | −2.9 | −1.7 | −4.1 | −2.3 | −5.7 | −3.7 | −5.9 |
| 4 | 3.5 | 5.7 | −2.7 | −2.3 | −2.5 | −3.2 | −3.1 | −4.4 | −3.3 | −4.8 |
| 5 | 10.2 | 11.4 | 2.8 | −6.3 | 1.1 | −7.0 | −7.7 | −10.5 | −8.4 | −12.5 |
| 6 | 5.5 | 3.0 | −2.1 | −2.4 | −2.8 | −4.5 | −4.5 | −4.9 | −5.4 | −6.1 |
| 7 | 8.4 | 11.7 | 0.1 | −4.4 | −0.9 | −8.2 | −3.0 | −9.4 | −7.2 | −10.4 |
| 8 | 3.0 | 2.9 | 0.3 | −1.5 | −0.3 | −1.6 | −1.3 | −1.4 | −2.0 | −2.0 |
| 9 | 9.9 | 11.3 | −1.3 | −1.7 | −2.2 | −4.1 | −3.4 | −7.5 | −7.7 | −10.4 |
| 10 | 3.3 | 6.0 | 1.6 | −3.2 | −2.2 | −3.7 | −3.1 | −4.4 | −3.9 | −4.2 |
| 11 | 3.3 | 5.5 | −3.4 | −2.9 | −3.5 | −3.7 | −3.5 | −6.3 | −4.2 | −6.9 |
| 12 | 3.0 | 3.6 | −1.7 | −2.2 | −2.0 | −2.2 | −2.5 | −3.1 | −2.6 | −2.8 |
| 13 | 13.4 | 8.6 | −3.8 | −3.0 | −5.1 | −3.6 | −10.3 | −5.7 | −10.7 | −6.3 |
| 14 | 1.5 | 6.2 | −2.6 | −1.7 | −3.1 | −3.1 | −0.8 | −4.7 | −2.7 | −6.0 |
| 15 | 3.9 | 4.7 | −1.0 | −1.4 | −2.3 | −2.0 | −2.5 | −3.4 | −3.5 | −3.5 |
| 16 | 3.4 | 2.2 | −2.8 | −1.6 | −3.9 | −1.5 | −4.0 | −2.4 | −5.3 | −1.9 |
| 17 | 6.8 | 6.2 | −1.2 | −3.8 | −2.7 | −4.5 | −4.6 | −4.8 | −5.2 | −5.1 |
| 18 | 2.9 | 2.1 | −3.6 | −1.8 | −3.4 | −2.8 | −3.5 | −2.5 | −3.7 | −3.0 |
| 19 | 5.2 | 6.6 | −2.5 | −3.6 | −3.8 | −4.4 | −4.4 | −4.9 | −6.2 | −7.7 |
| 20 | 3.3 | 2.8 | −3.8 | −4.7 | −3.1 | −5.3 | −2.9 | −5.1 | −6.1 | −6.1 |
| Average | 5.4 | 5.9 | −1.5 | −2.9 | −2.6 | −4.0 | −3.9 | −5.2 | −5.3 | −5.9 |
| S.D. | 3.1 | 2.9 | 1.8 | 1.2 | 1.5 | 1.8 | 2.1 | 2.3 | 2.3 | 2.8 |
| Median | 3.7 | 5.8 | −1.7 | −2.8 | −2.8 | −3.9 | −3.4 | −4.9 | −5.2 | −6.0 |

untr. subjects not receiving A
A composition as defined in Table 12

The solution of 5% SDS in water induced in all subjects after 1 week of washing a measurable and overall statistically significant increase in TEWL in both test areas (FIG. 10c). During the following application phase, TEWL was found to decrease in both the untreated and product treated test areas. The decrease in the product treated test area was found to be statistically significant larger after 1, 2 and 4 days of treatment as compared the changes in the untreated control area with “after SDS” taken as baseline (p<0.05) (FIG. 10c). On days 4 (area treated with test product) and 7 (untreated control area), TEWL was found (in statistical terms, p<0.05) to have returned to its original level as compared to the respective initial condition.

TABLE 32
Tewameter readings for transepidermal water loss after 1 week
SDS to start (end of regeneration) - protection (delta
Tewameter readings (g/hm²))

after 1 week
SDS t1-t0

| | untr. | A |
|---|---|---|
| 1 | 10.2 | 6.6 |
| 2 | 3.1 | 0.7 |
| 3 | 1.6 | 0.3 |
| 4 | 3.9 | 5.5 |
| 5 | 6.5 | 3.5 |
| 6 | 4.7 | 1.5 |
| 7 | 8.1 | 9.4 |
| 8 | 7.2 | 5.0 |
| 9 | 6.2 | 5.7 |
| 10 | 3.5 | 1.1 |
| 11 | 3.1 | 3.6 |
| 12 | 2.5 | 3.0 |
| 13 | 8.8 | 3.0 |
| 14 | 1.4 | 3.8 |
| 15 | 3.9 | 3.1 |
| 16 | 3.2 | 1.1 |
| 17 | 7.1 | 6.0 |
| 18 | 2.4 | 0.8 |
| 19 | 5.4 | 4.9 |
| 20 | 3.0 | 2.9 |
| Average | 4.8 | 3.6 |
| S.D. | 2.5 | 2.4 |
| Median | 3.9 | 3.3 |

untr. subjects not receiving A
A composition as defined in Table 12

7 days after the discontinuation of SDS washing, it was resumed again in addition to the product application (SDS washing 1 hour after the respective product application) and a repeated statistically significant increase in TEWL was observed both in the untreated and product treated test areas (FIG. 10d). The increase in the test product treated area was found to be statistically significantly (p<0.05) smaller than the increase in the untreated, SDS washed, control area.

The pharmaceutical composition according to the present invention has both a statistically significant regeneration effect and protection effect against the damaging properties of the surfactant SDS with regards to its effect on the barrier function of the skin (i.e. to accelerate recovery from barrier damage and to protect from induced barrier damage).

### Example 3

### Materials and Methods

Dermal fibroblast cells were grown in Advanced MEM supplemented with antibiotics (penicillin-streptomycin), 5% fetal bovine serum and L-glutamine (2 mmol/l) at 37° C. in a CO₂ incubator (5% CO₂, 95% air, 95% relative humidity. Cells were detached with Tryple™select. For experiments the cells were seeded in 96-well microtitre plates at a density 5×10³ cells/well (5×10⁴ cells/ml). Two hours after seeding, when the cells attached to the surface, the medium was replaced with the medium supplemented with vitamins (ACE), valsartan or their combination. The cells were incubated till the next day and then treated with H₂O₂ (1 mmol) for 15 minutes. After the treatment with H₂O₂ the cells were washed three times with PBS and than incubated in medium for 24 hours. Then the MTS viability test was performed.

### Results:

TABLE 33
Experimental data of dermal fibroblast survival
under oxidative stress:

| | Valsartan | ACE | Valsartan + ACE |
|---|---|---|---|
| % of survival after | 79% | 81% | 88% |
| treatment with H₂O₂ | | | |

Treatment with H₂O₂ resulted in around 75% survival of fibroblasts. Cell preincubation in a medium containing valsartan alone in concentration 1.0 μmol resulted in a significant improvement of cell survival reaching the level of 79% (P<0.05 vs. control). Cell preincubation in a medium containing vitamins ACE in the following concentrations: 1 μmol (vitamin A), 100 μmol (vitamin C) and 10 μmol (vitamin E) resulted in significant improvement of cell survival reaching the similar level of 81% (P<0.05 vs. control). There was no statistical difference in cell survival between fibroblasts pretreated either with vitamins ACE or with valsartan.

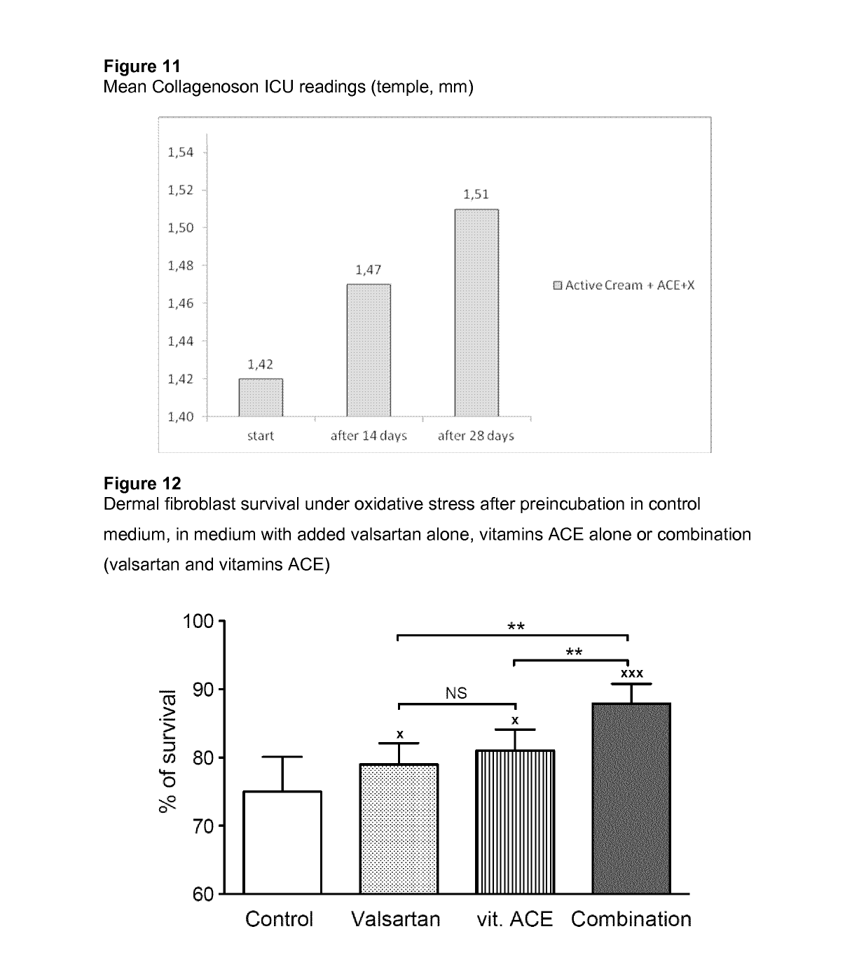

Cell preincubation in a medium to which a combination of valsartan and vitamins ACE (both in concentrations as used separately) was added, resulted in a substantial improvement of cell survival reaching the level of 88% (FIG. 12). This improvement was significantly higher than those obtained with either valsartan (P<0.01) or vitamins ACE (P<0.01).

In summary, these experiments show that the combination of valsartan and vitamins ACE is more effective in reducing oxidative stress in dermal fibroblasts indicated by increased cell survival rate than valsartan alone or vitamins ACE alone.

1. A topical composition comprising at least one angiotensin II receptor antagonist and at least one antioxidant.

2. A topical composition comprising at least one angiotensin II receptor antagonist and at least one antioxidant, wherein skin properties and/or characteristics are improved after application of the composition to skin for at least 14 days compared to the skin properties without application of the composition.

3. A topical composition comprising at least one angiotensin II receptor antagonist and at least one antioxidant, wherein symptoms of skin diseases and/or disorders are reduced after administration of the composition to skin for at least 14 days compared to the symptoms of skin diseases and/or disorders without administration of the composition.

4. The topical composition according to any one of claims 1 to 3, wherein the topical composition is for application or administration to apparently healthy human skin.

5. The topical composition according to any one of claims 1 to 4, wherein the topical composition is for application or administration to skin, preferably human skin, more preferably human facial skin.

6. The topical composition according to any one of claims 1 to 5, wherein the topical composition is for application or administration to the skin around the eyes.

7. The topical composition according to any one of claims 1 to 6, wherein the angiotensin II receptor antagonist is selected from the group consisting of azilsartan, losartan, eprosartan, irbesartan, olmesartan, candesartan, valsartan and telmisartan and any salts or esters thereof and any combinations of the foregoing.

8. The topical composition according to any one of claims 1 to 7, wherein the angiotensin II receptor antagonist is selected from the group consisting of azilsartan, losartan, telmisartan, olmesartan, candesartan and valsartan and any salts or esters thereof and any combinations of the foregoing.

9. The topical composition according to any one of claims 1 to 8, wherein the angiotensin II receptor antagonist is selected from the group consisting of losartan, telmisartan, azilsartan, candesartan and valsartan and any salts or esters thereof and any combinations of the foregoing.

10. The topical composition according to any one of claims 1 to 9, wherein the angiotensin II receptor antagonist is selected from the group consisting of losartan, valsartan and any salts or esters thereof and any combinations thereof.

11. The topical composition according to any one of claims 1 to 10, wherein the angiotensin II receptor antagonist is losartan or any salt thereof, preferably losartan potassium.

12. The topical composition according to any one of claims 1 to 11, wherein the angiotensin II receptor antagonist is valsartan or any salt thereof, preferably valsartan.

13. The topical composition according to any one of claims 1 to 12, wherein the angiotensin II receptor antagonist is present in an amount, which does not result in substantially lowering the systolic blood pressure or diastolic blood pressure upon topical application or administration to skin.

14. The topical composition according to any one of claims 1 to 13, wherein the angiotensin II receptor antagonist is present in an amount, which does not result in lowering the systolic blood pressure by more than about 15%, preferably not more than about 5%, more preferably not more than about 2%, most preferably not more than about 1% upon topical application or administration to skin.

15. The topical composition according to any one of claims 1 to 14, wherein the angiotensin II receptor antagonist is present in an amount, which does not result in lowering the systolic blood pressure by not more than about 15%, preferably not more than about 5%, more preferably not more than about 2%, most preferably not more than about 1% upon topical application or administration to skin, when the topical composition is applied or administered twice daily for 14, 21, 28 or 42 days in an amount of about 2 mg/cm².

16. The topical composition according to any one of claims 1 to 15, wherein the angiotensin II receptor antagonist is present in an amount, which does not result in lowering the diastolic blood pressure by more than about 15%, preferably not more than about 5%, more preferably not more than about 2%, most preferably not more than about 1% upon topical application or administration to skin.

17. The topical composition according to any one of claims 1 to 16, wherein the angiotensin II receptor antagonist is present in an amount, which does not result in lowering the diastolic blood pressure by more than about 15%, preferably not more than about 5%, more preferably not more than about 2%, most preferably not more than about 1% upon topical application or administration to skin, when the topical composition is applied or administered twice daily for 14, 21, 28 or 42 days in an amount of about 2 mg/cm².

18. The topical composition according to any one of claims 1 to 17, wherein the angiotensin II receptor antagonist is present in an amount of from about 0.1 to about 10 wt.-%, preferably from about 0.5 to about 5 wt.-%, more preferably from about 1 to about 3 wt.-%, most preferably in an amount of about 1.7 to about 2.3 wt.-% based on the total weight of the topical composition.

19. The topical composition according to any one of claims 1 to 11 or 13 to 18, wherein the angiotensin II receptor antagonist is losartan or any salt thereof, which is present in an amount of from about 0.1 to about 10 wt.-%, preferably from about 0.5 to about 5 wt.-%, more preferably from about 1 to about 3 wt.-%, most preferably in an amount of about 1.7 to about 2.3 wt.-% based on the total weight of the topical composition.

20. The topical composition according to any one of claims 1 to 10 or 12 to 18, wherein the angiotensin II receptor antagonist is valsartan or any salt thereof, which is present in an amount of from about 0.1 to about 10 wt.-%, preferably from about 0.5 to about 5 wt.-%, more preferably from about 1 to about 3 wt.-%, most preferably in an amount of about 1.7 to about 2.3 wt.-% based on the total weight of the topical composition.

21. The topical composition according to any one of claims 1 to 20, wherein the antioxidant is selected from the group consisting of beta-carotene, ascorbic acid, retinol, retinyl palmitate, tocopherol, tocopherol acetate, sodium ascorbyl phosphate, coenzyme Q10, resveratrol, magnesium ascorbyl phosphate, ascorbic acid-2-glycoside, butylated hydroxyanisole, any salts thereof and any analogues thereof and any combinations of the foregoing,

22. The topical composition according to any one of claims 1 to 21, wherein the antioxidant is selected from the group consisting of beta-carotene, ascorbic acid, tocopherol, tocopherol acetate, sodium ascorbyl phosphate, retinol, retinyl palmitate, coenzyme Q10 and resveratrol and any salts thereof and/or any analogue thereof and any combinations of the foregoing, preferably selected from the group consisting of tocopherol, tocopherol acetate, ascorbic acid, sodium ascorbyl phosphate, retinol, retinyl palmitate, coenzyme Q10, resveratrol and any combinations of the foregoing.

23. The topical composition according to any one of claims 1 to 22, wherein the antioxidant is present in an amount of from about 2 to about 20 wt.-%, preferably from about 5 to about 15 wt.-% and more preferably from about 8 to about 15 wt.-% based on the total weight of the topical composition.

24. The topical composition according to any one of claims 1 to 23, wherein the antioxidant comprises any one of vitamin C, vitamin A, vitamin E, any salts, analogues thereof, or any combinations of the foregoing.

25. The topical composition according to any one of claims 1 to 24, wherein the antioxidant is selected from the group consisting of vitamin C, vitamin A, vitamin E, any salts and analogues thereof, and any combinations of the foregoing.

26. The topical composition according to any one of claims 1 to 25, wherein the antioxidant is a combination of vitamin C, vitamin A, and vitamin E.

27. The topical composition according to any one of claims 1 to 26, wherein vitamin C is present in an amount of from about 1 to about 10 wt.-%, preferably from about 2 to about 8 wt.-%, more preferably from about 4 to about 6 wt.-%, most preferably about 5 wt.-% based on the total weight of the topical composition.

28. The topical composition according to any one of claims 1 to 27, wherein vitamin A is present in an amount of from about 1 to about 10 wt.-%, preferably from about 2 to about 8 wt.-%, more preferably from about 4 to about 6 wt.-%, most preferably about 5 wt.-% based on the total weight of the topical composition.

29. The topical composition according to any one of claims 1 to 28, wherein vitamin E is present in an amount of from about 1 to about 10 wt.-%, preferably from about 2 to about 8 wt.-%, more preferably from about 4 to about 6 wt.-%, most preferably about 5 wt.-% based on the total weight of the topical composition.

30. The topical composition according to any one of claims 1 to 25, wherein the antioxidant is a combination of sodium ascorbyl phosphate (sodium vitamin C phosphate), retinyl palmitate (vitamin A palmitate), tocopherol (vitamin E) and tocopherol acetate (vitamin E acetate).

31. The topical composition according to any one of claims 1 to 25 or 30, wherein sodium ascorbyl phosphate (sodium vitamin C phophate) is present in an amount of from about 0.2 to about 8 wt.-%, preferably from about 0.4 to about 6 wt.-%, more preferably from about 0.6 to about 4 wt.-%, most preferably from about 0.8 to about 2 wt.-% based on the total weight of the topical composition.

32. The topical composition according to any one of claims 1 to 25, 30 or 31, wherein retinyl palmitate (vitamin A palmitate) is present in an amount of from about 0.01 to about 5 wt.-%, preferably from about 0.05 to about 3 wt.-%, more preferably from about 0.08 to about 1 wt.-%, most preferably from about 0.1 to about 0.5 wt.-% based on the total weight of the topical composition.

33. The topical composition according to any one of claims 1 to 25 or 30 to 32, wherein tocopherol (vitamin E) is present in an amount of from about 0.01 to about 5 wt.-%, preferably from about 0.05 to about 3 wt.-%, more preferably from about 0.08 to about 1 wt.-%, most preferably from 0.1 to 0.5 wt.-% based on the total weight of the topical composition.

34. The topical composition according to any one of claims 1 to 25 or 30 to 33, wherein tocopherol acetate (vitamin E acetate) is present in an amount of from about 1 to about 10 wt.-%, preferably from about 2 to about 8 wt.-%, more preferably from about 3 to about 7 wt.-%, most preferably about 4 to about 6 wt.-% based on the total weight of the topical composition.

35. The topical composition according to any one of claims 1 to 24, wherein the antioxidant comprises coenzyme Q10, resveratrol, or a combination thereof.

36. The topical composition according to any one of claims 1 to 35, wherein the angiotensin II receptor antagonist and the antioxidant are present in the topical composition in an amount ratio of from about 1:2 to about 1:15, preferably from about 1:5 to about 1:10, more preferably from about 1:6 to about 1:9, most preferably about 1:7.5.

37. The topical composition according to any one of claims 1 to 35, wherein the angiotensin II receptor antagonist and the antioxidant are present in the topical composition in an amount ratio of from about 2:1 to about 1:10, preferably from about 1:1 to 1:10, more preferably from about 1:1 to 1:6, most preferably from about 1:2 to about 1:5.

38. The topical composition according to any one of claims 1 to 11, 13 to 19, 21 to 29, 36, or 37, wherein the angiotensin II receptor antagonist is losartan or any salt thereof and the antioxidant is selected from the group consisting of vitamin C, vitamin A, vitamin E, any salts and analogues thereof, and combinations of the foregoing.

39. The topical composition according to any one of claims 1 to 11, 13 to 19, 21 to 29, or 36 to 38, wherein the angiotensin II receptor antagonist is losartan or any salt thereof and the antioxidant is a combination of vitamin C, vitamin A, and vitamin E.

40. The topical composition according to any one of claims 1 to 11, 13 to 19, 21 to 29, or 36 to 39, wherein the angiotensin II receptor antagonist is losartan potassium and is present in an amount of from about 1 to about 10 wt.-%, preferably from about 1 to about 5 wt.-%, more preferably from about 1 to about 3 wt.-%, most preferably in an amount of from about 1.7 wt.-% to about 2.3 wt.-% relative to the total weight of the topical composition, and the antioxidant is a combination of vitamin C, vitamin A, vitamin E.

41. The topical composition according to any one of claims 1 to 11, 13 to 19, 21 to 29, or 36 to 40, wherein the angiotensin II receptor antagonist is losartan potassium, and is present in an amount of from about 1 to about 10 wt.-%, preferably from about 1 to about 5 wt.-%, more preferably from about 1 to about 3 wt.-%, most preferably in an amount of from about 1.7 wt.-% to about 2.3 wt.-% relative to the total weight of the topical composition, and the antioxidant is a combination of vitamin C, vitamin A, and vitamin E, and wherein
a) vitamin C is present in an amount of from about 1 to about 10 wt.-%, preferably from about 2 to about 8 wt.-%, more preferably from about 4 to about 6 wt.-%, most preferably about 5 wt.-% based on the total weight of the topical composition,
wherein
b) vitamin A is present in an amount of from about 1 to about 10 wt.-%, preferably from about 2 to about 8 wt.-%, more preferably from about 4 to about 6 wt.-%, most preferably about 5 wt.-% based on the total weight of the topical composition,
and wherein
c) vitamin E is present in an amount of from about 1 to about 10 wt.-%, preferably from about 2 to about 8 wt.-%, more preferably from about 4 to about 6 wt.-%, most preferably about 5 wt.-% based on the total weight of the topical composition.

42. The topical composition according to any one of claims 1 to 10, 12 to 18, 20 to 25, 30 to 34, 36 or 37, wherein the angiotensin II receptor antagonist is valsartan or any salt thereof and the antioxidant is selected from the group consisting of vitamin C, vitamin A, vitamin E, any salts or analogues thereof, and combinations of the foregoing.

43. The topical composition according to any one of claims 1 to 10, 12 to 18, 20 to 25, 30 to 34, 36, 37, or 42, wherein the angiotensin II receptor antagonist is valsartan or any salt thereof and the antioxidant is selected from the group consisting of sodium ascorbyl phosphate (sodium vitamin C phosphate), retinyl palmitate (vitamin A palmitate), tocopherol (vitamin E) and tocopherol acetate (vitamin E acetate), and any combinations of the foregoing.

44. The topical composition according to any one of claims 1 to 10, 12 to 18, 20 to 25, 30 to 34, 36, 37, 42, or 43, wherein the angiotensin II receptor antagonist is valsartan and is present in an amount of from about 1 to about 10 wt.-%, preferably from about 1 to about 5 wt.-%, more preferably from about 1 to about 3 wt.-%, most preferably in an amount of from about 1.7 wt.-% to about 2.3 wt.-% relative to the total weight of the topical composition, and the antioxidant is a combination of sodium ascorbyl phosphate (sodium vitamin C phosphate), retinyl palmitate (vitamin A palmitate), tocopherol (vitamin E) and tocopherol acetate (vitamin E acetate).

45. The topical composition according to any one of claims 1 to 10, 12 to 18, 20 to 25, 30 to 34, 36, 37, or 42 to 44, wherein the angiotensin II receptor antagonist is valsartan, and is present in an amount of from about 1 to about 10 wt.-%, preferably from about 1 to about 5 wt.-%, more preferably from about 1 to about 3 wt.-%, most preferably in an amount of from about 1.7 wt.-% to about 2.3 wt.-% relative to the total weight of the topical composition, and the antioxidant is a combination of sodium ascorbyl phosphate (sodium vitamin C phosphate), retinyl palmitate (vitamin A palmitate), tocopherol (vitamin E) and tocopherol acetate (vitamin E acetate), wherein
a) sodium ascorbyl phosphate (sodium vitamin C phosphate) is present in an amount of from about 0.2 to about 8 wt.-%, preferably from about 0.4 to about 6 wt.-%, more preferably from about 0.6 to about 4 wt.-%, most preferably from about 0.8 to about 2 wt.-% based on the total weight of the topical composition,
wherein
b) retinyl palmitate (vitamin A palmitate) is present in an amount of from about 0.01 to about 5 wt.-%, preferably from about 0.05 to about 3 wt.-%, more preferably from about 0.08 to about 1 wt.-%, most preferably from about 0.1 to about 0.5 wt.-% based on the total weight of the topical composition,
wherein
c) tocopherol (vitamin E) is present in an amount of from about 0.01 to about 5 wt.-%, preferably from about 0.05 to about 3 wt.-%, more preferably from about 0.08 to about 1 wt.-%, most preferably from about 0.1 to about 0.5 wt.-% based on the total weight of the topical composition,
and wherein
d) tocopherol acetate (vitamin E acetate) is present in an amount of from about 1 to about 10 wt.-%, preferably from about 2 to about 8 wt.-%, more preferably from about 3 to about 7 wt.-%, most preferably from about 4 to about 6 wt.-% based on the total weight of the topical composition.

46. The topical composition according to any one of claims 1 to 45, wherein the topical composition further comprises a triglyceride, preferably caprylic/capric triglyceride.

47. The topical composition according to any one of claims 1 to 46, wherein the topical composition further comprises at least one further ingredient selected from the group consisting of 2-hydroxy-4-methoxybenzophenone, 1-phenyl-3(4′-isopropylphenyl)propane-1,3-dione, macrogel stearate, cetylstearyl alcohol, 1,2-propandiole, and vaseline.

48. The topical composition according to any one of claims 1 to 47, wherein the topical composition further comprises at least one further ingredient selected from the group consisting of cetearyl alcohol, cetearyl glucoside, alcohol denat., polyacrylamide, C₁₃₋₁₄ isoparaffin, laureth-7, glycerin, dicaprylyl carbonate, dicaprylyl ether, propylheptyl caprylate, dimethicone, stearic acid, cetyl alcohol, stearyl alcohol, triethanolamine, phenoxyethanol, ethylhexylglycerin, disodium EDTA, perfume, and water.

49. The topical composition according to any one of claims 1 to 48, wherein the topical composition further comprises at least one further cosmetic ingredient according to the INCI system selected from the group consisting of abrasives, absorbents, additives, anticorrosives, anti-dandruff agents, anti-foaming agents, antimicrobials, antiperspirants, antistatic agents, binders, biological additives, bleaching agents, botanicals, buffering agents, chelating agents, colorants, denaturants, deodorants, depilating agents, emollients, emulsifiers, emulsion stabilizers, film-formers, hair dyes, humectants, opacifiers, oral care agents, oxidizing agents, preservatives, propellants, reducing agents, solvents, surfactants, UV absorbers, viscosity enhancers and any combinations of the foregoing.

50. The topical composition according to any one of claims 1 to 49, wherein the topical composition further comprises at least one further ingredient selected from the group consisting of penetration enhancers, thickeners, stiffening agents, solvents, emulsifiers, emollients, preservatives, buffers, vehicles, fragrances, coloring agents, chelating agents, humectants and any combinations of the foregoing.

51. The topical composition according to any one of claims 1 to 50, wherein the topical composition further comprises at least one further ingredient selected from the group consisting of exfoliants, anti-acne agents, anti-aging agents, anti-dark circles agents, anti-wrinkle agents, conditioners, hair repair agents, nourishing agents, pearlizers, perfume ingredients, self-tanners, skin-lightening agents, sunscreens and any combinations of the foregoing.

52. The topical composition according to any one of claims 1 to 51, wherein the topical composition further comprises at least one excipient.

53. The topical composition according to any one of claims 1 to 52, wherein the topical composition does not comprise an UVA and/or UVB filter.

54. The topical composition according to any one of claims 1 to 53, wherein the topical composition does not comprise a UV filter.

55. The topical composition according to any one of claims 1 to 54, wherein the topical composition does not comprise a barrier compound.

56. The topical composition according any one of claims 1 to 55, wherein the topical composition does not comprise a barrier compound, wherein the barrier compound acts as a sunscreen.

57. The topical composition according to any one of claims 1 to 56, wherein the topical composition does not comprise a barrier compound selected from 2-hydroxy-4-methoxybenzophenone and/or 1-phenyl-3-(4′-isopropylphenyl)propane-1,3-dione.

58. The topical composition according to any one of claims 1 to 10, 12 to 18, 20 to 25, 30 to 34, 36, 37, or 42 to 57, wherein the topical composition comprises valsartan or any salt thereof salt thereof as angiotensin II receptor antagonist, and does not comprise a barrier compound selected from 2-hydroxy-4-methoxybenzophenone and/or 1-phenyl-3-(4′-isopropylphenyl)propane-1,3-dione.

59. The topical composition according to any one of claims 1 to 57, wherein the topical composition essentially consists of
a) at least one angiotensin II receptor antagonist, preferably losartan or any salt thereof
b) at least one antioxidant, and
c) at least one further excipient selected from the group consisting of 2-hydroxy-4-methoxybenzophenone, 1-phenyl-3(4′-isopropylphenyl)propane-1,3-dione, macrogel stearate, cetylstearyl alcohol, 1,2-propandiole, and vaseline.

60. The topical composition according to any one of claims 1 to 58, wherein the topical composition essentially consists of
a) at least one angiotensin II receptor antagonist, preferably valsartan or any salt thereof
b) at least one antioxidant, and
c) at least one further excipient selected from the group consisting of cetearyl alcohol, cetearyl glucoside, alcohol denat., polyacrylamide, C₁₃₋₁₄ isoparaffin, laureth-7, glycerin, dicaprylyl carbonate, dicaprylyl ether, propylheptyl caprylate, dimethicone, stearic acid, cetyl alcohol, stearyl alcohol, triethanolamine, phenoxyethanol, ethylhexylglycerin, disodium EDTA, perfume, and water.

61. The topical composition according to any one of claims 1 to 60, wherein the topical composition is in the form of
a) liquids, preferably selected from the group consisting of emulsions, suspensions, solutions, sprays or shampoos,
b) semi-solids, preferably selected from the group consisting of colloids, foams, ointments, pastes, creams or gels, or
c) solids, preferably selected from the group consisting of powders, aerosols, patches, gauzes, compress, tapes, sticks or tablets for topical application or administration.

62. The topical composition according to any one of claims 1 to 61, wherein the topical composition is in the form of a cream, a lotion or a skin patch, preferably a cream.

63. The topical composition according to any one of claims 1 to 62, wherein the topical composition is in the form of a face cream.

64. The topical composition according to any one of claims 1 to 62, wherein the topical composition is in the form of an eye cream.

65. The topical composition according to any one of claims 1 to 64, wherein the topical composition is applied or administered to skin at least once in a week, preferably at least once in two days, more preferably at least once daily, most preferably at least twice daily.

66. The topical composition according to any one of claims 1 to 65, wherein the topical composition is applied or administered to skin for at least 1 day, preferably at least 2 days, more preferably at least 7 days.

67. The topical composition according to any one of claims 1 to 66, wherein the topical composition is applied or administered to skin for at least 14 days, preferably for at least 28 days.

68. The topical composition according to any one of claims 1 to 67, wherein the topical composition is applied or administered to skin at least twice daily for at least 14 days, preferably for at least 28 day.

69. The topical composition according to any one of claims 1 to 68, wherein the topical composition is applied or administered to skin for at least 21 days, preferably at least 42 days.

70. The topical composition according to any one of claims 1 to 69, wherein the topical composition is applied or administered to skin at least twice daily for at least 21 days, preferably at least 42 days.

71. The topical composition according to any one of claims 1 to 70, wherein the topical composition is applied or administered to skin in an amount of at least about 0.1 mg/cm², preferably at least about 0.5 mg/cm², more preferably at least about 1 mg/cm², most preferably at least about 2 mg/cm².

72. The topical composition according to any one of claims 1 to 71, wherein the topical composition is applied or administered to skin in an amount of at least about 2 mg/cm² at least twice daily for at least 14 days, preferably 28 days, or in an amount of at least about 2 mg/cm² at least twice daily for at least 21 days, preferably 42 days.

73. The topical composition according to any one of claims 1 to 72, wherein the skin firmness is increased after application of the topical composition compared to the skin firmness without application of the topical composition.

74. The topical composition according to any one of claims 1 to 73, wherein the skin firmness is increased by at least 1%, preferably by at least 3%, more preferably by at least 5%, most preferably by at least 8% after application of the topical composition, preferably after application for at least 21 days, more preferably after application for at least 14 days, compared to the skin firmness without application of the topical composition.

75. The topical composition according to any one of claims 1 to 74, wherein the mean skin firmness is increased by at least 1%, preferably by at least 3%, more preferably by at least 5%, most preferably by at least 8% after application of the topical composition, preferably after application for at least 21 days, more preferably after application for at least 14 days, compared to the mean skin firmness without application of the topical composition.

76. The topical composition according to any one of claims 1 to 75, wherein the skin firmness is increased by at least 5%, preferably by at least 10%, more preferably by at least 12%, most preferably by at least 14% after application of the topical composition, preferably after application for at least 42 days, more preferably after application for at least 28 days, compared to the skin firmness without application of the topical composition.

77. The topical composition according to any one of claims 1 to 76, wherein the mean skin firmness is increased by at least 5%, preferably by at least 10%, more preferably by at least 12%, most preferably by at least 14% after application of the topical composition, preferably after application for at least 42 days, more preferably after application for at least 28 days, compared to the mean skin firmness without application of the topical composition.

78. The topical composition according to any one of claims 1 to 77, wherein the skin elasticity is increased after application of the topical composition compared to the skin elasticity without application of the topical composition.

79. The topical composition according to any one of claims 1 to 78, wherein the skin elasticity is increased by at least 1%, preferably by at least 3%, more preferably by at least 5%, most preferably by at least 6% after application of the topical composition, preferably after application for at least 21 days, more preferably after application for at least 14 days, compared to the skin elasticity without application of the topical composition.

80. The topical composition according to any one of claims 1 to 79, wherein the mean skin elasticity is increased by at least 1%, preferably by at least 3%, more preferably by at least 5%, most preferably by at least 6% after application of the topical composition, preferably after application for at least 21 days, more preferably after application for at least 14 days, compared to the mean skin elasticity without application of the topical composition.

81. The topical composition according to any one of claims 1 to 80, wherein the skin elasticity is increased by at least 3%, preferably by at least 5%, more preferably by at least 7%, most preferably by at least 9% after application of the topical composition, preferably after application for at least 42 days, more preferably after application for at least 28 days, compared to the skin elasticity without application of the topical composition.

82. The topical composition according to any one of claims 1 to 81, wherein the mean skin elasticity is increased by at least 3%, preferably by at least 5%, more preferably by at least 7%, most preferably by at least 9% after application of the topical composition, preferably after application for at least 42 days, more preferably after application for at least 28 days, compared to the mean skin elasticity without application of the topical composition.

83. The topical composition according to any one of claims 1 to 82, wherein the skin fatigue resistance is increased after application of the topical composition compared to the skin fatigue resistance without application of the topical composition.

84. The topical composition according to any one of claims 1 to 83, wherein the skin fatigue resistance is increased by at least 3%, preferably by at least 5%, more preferably by at least 7%, most preferably by at least 9% after application of the topical composition, preferably after application for at least 21 days, more preferably after application for at least 14 days, compared to the skin fatigue resistance without application of the topical composition.

85. The topical composition according to any one of claims 1 to 84, wherein the mean skin fatigue resistance is increased by at least 3%, preferably by at least 5%, more preferably by at least 7%, most preferably by at least 9% after application of the topical composition, preferably after application for at least 21 days, more preferably after application for at least 14 days, compared to the mean skin fatigue resistance without application of the topical composition.

86. The topical composition according to any one of claims 1 to 85, wherein the skin fatigue resistance is increased by at least 4%, preferably by at least 6%, more preferably by at least 8%, most preferably by at least 10% after application of the topical composition, preferably after application for at least 42 days, more preferably after application for at least 28 days, compared to the skin fatigue resistance without application of the topical composition.

87. The topical composition according to any one of claims 1 to 86, wherein the mean skin fatigue resistance is increased by at least 4%, preferably by at least 6%, more preferably by at least 8%, most preferably by at least 10% after application of the topical composition, preferably after application for at least 42 days, more preferably after application for at least 28 days, compared to the mean skin fatigue resistance without application of the topical composition.

88. The topical composition according to any one of claims 1 to 87, wherein the wrinkle depth, wrinkle area and wrinkle volume of wrinkles of skin, preferably of inner side of forearms or face, more preferably Crow's feet, are reduced after application of the topical composition compared to the wrinkle depth, area and volume of wrinkles of skin, preferably of inner side of forearms or face, more preferably Crow's feet, without application of the topical composition.

89. The topical composition according to any one of claims 1 to 88, wherein the wrinkle depth, preferably the wrinkle depth in Crow's feet, is reduced by at least 2%, preferably by at least 4%, more preferably by at least 6%, most preferably by at least 8% after application of the topical composition, preferably after application for at least 21 days, more preferably after application for at least 14 days, compared to the wrinkle depth without application of the topical composition.

90. The topical composition according to any one of claims 1 to 89, wherein the mean wrinkle depth, preferably the mean wrinkle depth in Crow's feet, is reduced by at least 2%, preferably by at least 4%, more preferably by at least 6%, most preferably by at least 8% after application of the topical composition, preferably after application for at least 21 days, more preferably after application for at least 14 days, compared to the mean wrinkle depth without application of the topical composition.

91. The topical composition according to any one of claims 1 to 90, wherein the wrinkle depth, preferably the wrinkle depth in Crow's feet, is reduced by at least 5%, preferably by at least 8%, more preferably by at least 12%, most preferably by at least 13% after application of the topical composition, preferably after application for at least 21 days, more preferably after application for at least 14 days, compared to the wrinkle depth without application of the topical composition.

92. The topical composition according to any one of claims 1 to 91, wherein the mean wrinkle depth, preferably the mean wrinkle depth in Crow's feet, is reduced by at least 5%, preferably by at least 8%, more preferably by at least 12%, most preferably by at least 15% after application of the topical composition, preferably after application for at least 42 days, more preferably after application for at least 28 days, compared to the mean wrinkle depth without application of the topical composition.

93. The topical composition according to any one of claims 1 to 92, wherein the wrinkle area, preferably the wrinkle area in Crow's feet, is reduced by at least 0.1%, preferably by at least 0.5%, more preferably by at least 1.5%, most preferably by at least 2.5% after application of the topical composition, preferably after application for at least 21 days, more preferably after application for at least 14 days, compared to the wrinkle area without application of the topical composition.

94. The topical composition according to any one of claims 1 to 93, wherein the mean wrinkle area, preferably the mean wrinkle area in Crow's feet, is reduced by at least 0.1%, preferably by at least 0.5%, more preferably by at least 1.5%, most preferably by at least 2.5% after application of the topical composition, preferably after application for at least 21 days, more preferably after application for at least 14 days, compared to the mean wrinkle area without application of the topical composition.

95. The topical composition according to any one of claims 1 to 94, wherein the wrinkle area, preferably the wrinkle area in Crow's feet, is reduced by at least 4%, preferably by at least 7%, more preferably by at least 10%, most preferably by at least 13% after application of the topical composition, preferably after application for at least 42 days, more preferably after application for at least 28 days, compared to the wrinkle area without application of the topical composition.

96. The topical composition according to any one of claims 1 to 95, wherein the mean wrinkle area, preferably the mean wrinkle area in Crow's feet, is reduced by at least 4%, preferably by at least 7%, more preferably by at least 10%, most preferably by at least 13% after application of the topical composition, preferably after application for at least 42 days, more preferably after application for at least 28 days, compared to the mean wrinkle area without application of the topical composition.

97. The topical composition according to any one of claims 1 to 96, wherein the wrinkle volume, preferably the wrinkle volume in Crow's feet, is reduced by at least 3%, preferably by at least 6%, more preferably by at least 9%, most preferably by at least 11% after application of the topical composition, preferably after application for at least 21 days, more preferably after application for at least 14 days, compared to the wrinkle volume without application of the topical composition.

98. The topical composition according to any one of claims 1 to 97, wherein the mean wrinkle volume, preferably the mean wrinkle volume in Crow's feet, is reduced by at least 3%, preferably by at least 6%, more preferably by at least 9%, most preferably by at least 11% after application of the topical composition, preferably after application for at least 21 days, more preferably after application for at least 14 days, compared to the mean wrinkle volume without application of the topical composition.

99. The topical composition according to any one of claims 1 to 98, wherein the wrinkle volume, preferably the wrinkle volume in Crow's feet is reduced by at least 8%, preferably by at least 14%, more preferably by at least 20%, most preferably by at least 25% after application of the topical composition, preferably after application for at least 42 days, more preferably after application for at least 28 days, compared to the wrinkle volume without application of the topical composition.

100. The topical composition according to any one of claims 1 to 99, wherein the mean wrinkle volume, preferably the mean wrinkle volume in Crow's feet is reduced by at least 8%, preferably by at least 14%, more preferably by at least 20%, most preferably by at least 25% after application of the topical composition, preferably after application for at least 42 days, more preferably after application for at least 28 days, compared to the mean wrinkle volume without application of the topical composition.

101. The topical composition according to any one of claims 1 to 100, wherein the oxidative stress is reduced after application of the topical composition compared to the oxidative stress without application of the topical composition.

102. The topical composition according to any one of claims 1 to 101, wherein the oxidative stress is reduced by at least 10%, preferably by at least 20%, more preferably by at least 27%, most preferably by at least 28% after application of the topical composition, preferably after a single application of the topical composition, compared to the oxidative stress without application of the topical composition.

103. The topical composition according to any one of claims 1 to 102, wherein the mean oxidative stress is reduced by at least 10%, preferably by at least 20%, more preferably by at least 27%, most preferably by at least 28% after application of the topical composition, preferably after a single application of the topical composition, compared to the mean oxidative stress without application of the topical composition.

104. The topical composition according to any one of claims 1 to 103, wherein the skin roughness is reduced after application of the topical composition of the present invention compared to the skin roughness without application of the topical composition.

105. The topical composition according to any one of claims 1 to 104, wherein the skin roughness is reduced by at least 5%, preferably by at least 8%, more preferably by at least 10%, most preferably by at least 12%, preferably after application for at least 21 days, preferably for at least 14 days, compared to the skin roughness without application of the topical composition.

106. The topical composition according to any one of claims 1 to 105, wherein the mean skin roughness is reduced by at least 5%, preferably by at least 8%, more preferably by at least 10%, most preferably by at least 12%, preferably after application for at least 21 days, preferably for at least 14 days, compared to the mean skin roughness without application of the topical composition.

107. The topical composition according to any one of claims 1 to 106, wherein the skin roughness is reduced by at least 8%, preferably by at least 10%, more preferably by at least 12%, most preferably by at least 13%, preferably after application for at least 42 days, preferably for at least 28 days, compared to the skin roughness without application of the topical composition.

108. The topical composition according to any one of claims 1 to 107, wherein the mean skin roughness is reduced by at least 8%, preferably by at least 10%, more preferably by at least 12%, most preferably by at least 13%, preferably after application for at least 42 days, preferably for at least 28 days, compared to the mean skin roughness without application of the topical composition.

109. The topical composition according to any one of claims 1 to 108, wherein the skin thickness is increased after application of the topical composition compared to the skin thickness without application of the topical composition.

110. The topical composition according to any one of claims 1 to 109, wherein the skin thickness is increased by at least 0.005 mm, preferably by at least 0.01 mm, more preferably by at least 0.03 mm, most preferably by at least 0.04 mm after application of the topical composition.

111. The topical composition according to any one of claims 1 to 110, wherein the skin thickness is increased by at least 0.005 mm, preferably by at least 0.01 mm, more preferably by at least 0.03 mm, after application for at least 21 days, more preferably after application for at least 14 days, compared to the skin thickness without application of the topical composition.

112. The topical composition according to any one of claims 1 to 111, wherein the skin thickness is increased by at least 0.01 mm, preferably by at least 0.03 mm, more preferably by at least 0.06 mm, most preferably by at least 0.08 mm after application of the topical composition, preferably after application for at least 42 days, more preferably after application for at least 28 days, compared to the skin thickness without application of the topical composition.

113. The topical composition according to any one of claims 1 to 112, wherein transepidermal water loss is reduced and/or prevented after application or administration of the topical composition compared to transepidermal water loss without application or administration of the topical composition.

114. The topical composition according to any one of claims 1 to 113, transepidermal water loss is reduced, indicated by a reduction of the transepidermal water loss, which is at least 2.5%, preferably at least 10%, more preferably at least 15%, most preferably at least 20% greater after application or administration of the topical composition for at least 1 day, preferably for at least 2 days, compared to the reduction of the transepidermal water loss without application or administration of the topical composition.

115. The topical composition according to any one of claims 1 to 114, wherein transepidermal water loss is prevented, indicated by an increase of the transepidermal water loss, which is at least 4% less, preferably 12% less, more preferably 19% less, most preferably 23% less after application or administration of the topical composition together with sodium dodecyl sulphate, for at least 1 day, preferably at least 2 days, compared to the increase of the transepidermal water loss after application or administration of sodium dodecyl sulphate without application of the topical composition.

116. The topical composition according to any one of claims 1 to 115, wherein symptoms of skin irritation and/or neurodermitis are reduced and/or prevented after administration of the topical composition compared to the symptoms without administration of the topical composition.

117. The topical composition according to any one of claims 1 to 116, wherein skin redness is reduced and/or prevented after administration of the topical composition compared to skin redness without administration of the topical composition.

118. The topical composition according to any one of claims 1 to 117, wherein skin redness is reduced, indicated by a reduction of the hue and/or color saturation on the red-green axis a*, which is at least 4.5%, preferably at least 10%, more preferably at least 15%, most preferably at least 20% greater after administration of the topical composition for at least 1 day, preferably 2 days, compared to the reduction of the hue and/or color saturation on the red-green axis a* without administration of the topical composition.

119. The topical composition according to any one of claims 1 to 118, wherein skin redness is prevented indicated by an increase of the hue and/or color saturation on the red-green axis a*, which is at least 6% less, preferably at least 20% less, more preferably at least 25% less, most preferably at least 28% less after administration of the topical composition together with sodium dodecyl sulphate for at least 1 day, preferably at least 7 days, compared to the increase of the hue and/or color saturation on the red-green axis a* after administration of sodium dodecyl sulphate without administration of the topical composition.

120. The topical composition according to any one of claims 1 to 115, wherein the topical composition is a cosmetic composition.

121. The topical composition according to any one of claims 1 to 115 or 120, wherein any salt in the topical composition is a cosmetically acceptable salt.

122. The topical composition according to any one of claims 1 to 115, 120 or 121, wherein any ester in the topical composition is a cosmetically acceptable ester.

123. The topical composition according to any one of claims 1 to 115, or 120 to 122 comprising an additional cosmetically active agent.

124. The topical composition according to any one of claims 1 to 115, or 120 to 123, wherein the topical composition does not comprise a barrier compound, in particular not a barrier compound selected from 2-hydroxy-4-methoxybenzophenone and/or 1-phenyl-3-(4′-isopropylphenyl)propane-1,3-dione.

125. Use of the topical composition according to any one of claims 1 to 115, or 120 to 123 for improving or maintaining the biomechanical properties of the skin.

126. Use according to claim 125 for improving or maintaining the biomechanical properties of the skin, wherein the biomechanical properties are selected from skin firmness, skin elasticity and/or skin fatigue resistance.

127. Use according to claim 125 or 126 for changing the biomechanical properties of the skin towards the firm-elastic optimum.

128. Use of the topical composition according to any one of claims 1 to 115, or 120 to 123 for decreasing wrinkle depth, wrinkle area and/or wrinkle volume.

129. Use according to claim 128 for decreasing wrinkle depth, wrinkle area, and/or wrinkle volume of wrinkles of skin, preferably of inner side of forearms or face, more preferably in Crow's feet.

130. Use of the topical composition according to any one of claims 1 to 115, or 120 to 123 for protecting skin from oxidative stress, preferably photooxidative stress.

131. Use according to claim 130 for protecting the skin from photooxidative stress induced by UV radiation, preferably UVA radiation, by increasing the elimination of free radicals formed under UV radiation, preferably UVA radiation.

132. Use of the topical composition according to any one of claims 1 to 115, or 120 to 123 for preventing or reducing skin roughness.

133. Use of the topical composition according to any one of claims 1 to 115, or 120 to 123 for maintaining or improving skin hydration.

134. Use of the topical composition according to any one of claims 1 to 115, or 120 to 123 for maintaining or improving skin thickness.

135. Use of the topical composition according to any one of claims 1 to 115, or 120 to 123 for
a) maintaining or improving the biomechanical properties of skin,
b) decreasing wrinkle depth, wrinkle area and/or wrinkle volume,
c) protecting skin from oxidative stress,
d) preventing or reducing skin roughness,
e) maintaining or improving skin hydration, and/or
f) maintaining or improving skin thickness.

136. Use of the topical composition according to any one of claims 1 to 115, or 120 to 123 for improving and/or maintaining the collagen structure of the skin.

137. Use of the topical composition according to any one of claims 1 to 115, or 120 to 123 for modulating, preferably maintaining and/or increasing, the formation of collagen in the skin.

138. The topical composition according to any one of claims 1 to 115, or 120 to 123 for use in improving or maintaining the biomechanical properties of the skin.

139. The topical composition according to any one of claims 1 to 115, 120 to 123 or 138 for use in improving or maintaining the biomechanical properties of the skin, wherein the biomechanical properties are selected from skin firmness, skin elasticity, and/or skin fatigue resistance.

140. The topical composition according to any one of claims 1 to 115, 120 to 123, 138 or 139 for use in changing the biomechanical properties of the skin towards the firm-elastic optimum.

141. The topical composition according to any one of claims 1 to 115, 120 to 123, or 138 or 140 for use in decreasing wrinkle depth, wrinkle area and/or wrinkle volume.

142. The topical composition according to any one of claims 1 to 115, 120 to 123, or 138 or 141 for use in decreasing wrinkle depth, area, and volume of wrinkles of skin, preferably of inner side of forearms or face, more preferably in Crow's feet.

143. The topical composition according to any one of claims 1 to 115, 120 to 123, or 138 or 142 for use in protecting skin from oxidative stress, preferably photooxidative stress.

144. The topical composition according to any one of claims 1 to 115, 120 to 123, or 138 or 143 for use in protecting skin from photooxidative stress induced by UV radiation, preferably UVA radiation, by increasing the elimination of free radicals formed under UV radiation, preferably UVA radiation.

145. The topical composition according to any one of claims 1 to 115, 120 to 123, or 138 or 144 for use in preventing or reducing skin roughness.

146. The topical composition according to any one of claims 1 to 115, 120 to 123, or 138 or 145 for use in maintaining or improving skin hydration.

147. The topical composition according to any one of claims 1 to 115, 120 to 123, or 138 or 146 for use in maintaining or improving skin thickness.

148. The topical composition according to any one of claims 1 to 115, 120 to 123, or 138 or 147 for use in
a) improving or maintaining the biomechanical properties of skin,
b) decreasing wrinkle depth, wrinkle area and/or wrinkle volume,
c) protecting skin from oxidative stress,
d) preventing or reducing skin roughness,
e) maintaining or improving skin hydration, and/or
f) maintaining or improving skin thickness.

149. The topical composition according to any one of claims 1 to 115, 120 to 123, or 138 or 148 for use in improving and/or maintaining the collagen structure of the skin.

150. The topical composition according to any one of claims 1 to 115, 120 to 123, or 138 or 149 for use in modulating, preferably maintaining and/or increasing, the formation of collagen in the skin.

151. Method for improving or maintaining the biomechanical properties of the skin comprising contacting the topical composition according to any one of claims 1 to 115, or 120 to 123 with skin.

152. Method according to claim 151, wherein the biomechanical properties are selected from skin firmness, skin elasticity, and/or skin fatigue resistance.

153. Method according to claim 151 or 152 for changing the biomechanical properties of the skin towards the firm-elastic optimum.

154. Method for decreasing wrinkle depth, wrinkle area, and/or wrinkle volume comprising contacting the topical composition according to any one of claims 1 to 115, or 120 to 123 with skin.

155. Method according to claim 154 for decreasing wrinkle depth, wrinkle area, and/or wrinkle volume of wrinkles of skin, preferably of inner side of forearms or face, more preferably Crow's feet.

156. Method for protecting skin from oxidative stress, preferably photooxidative stress, comprising contacting the topical composition according to any one of claims 1 to 115, or 120 to 123 with skin.

157. Method according to claim 156 for protecting the skin from photooxidative stress induced by UV radiation, preferably UVA radiation, by increasing the elimination of free radicals.

158. Method for preventing or reducing skin roughness comprising contacting the topical composition according to any one of claims 1 to 115, or 120 to 123 with skin.

159. Method for maintaining or improving skin hydration comprising contacting the topical composition according to any one of claims 1 to 115, or 120 to 123 with skin.

160. Method for maintaining or improving skin thickness comprising contacting the topical composition according to any one of claims 1 to 115, or 120 to 123 with skin.

161. Method for
a) improving or maintaining the biomechanical properties of skin,
b) decreasing wrinkle depth, wrinkle area and/or wrinkle volume,
c) protecting skin from oxidative stress,
d) preventing or reducing skin roughness,
e) maintaining or improving skin hydration, and/or
f) maintaining or improving skin thickness
comprising contacting the topical composition according to any one of claims 1 to 115, or 120 to 123 with skin.

162. Method for improving and/or maintaining the collagen structure of the skin comprising contacting the topical composition according to any one of claims 1 to 115, or 120 to 123 with skin.

163. Method for modulating, preferably maintaining and/or increasing, the formation of collagen in the skin comprising contacting the topical composition according to any one of claims 1 to 115, or 120 to 123 with skin.

164. The topical composition according to any one of claims 1 to 72 or 113 to 119, wherein the topical composition is a pharmaceutical composition.

165. The topical composition according to any one of claims 1 to 72, 113 to 119, or 164 wherein any salt in the topical composition is a pharmaceutically acceptable salt.

166. The topical composition according to any one of claims 1 to 72, 113 to 119, 164 or 165, wherein any ester in the topical composition is a pharmaceutically acceptable ester.

167. The topical composition according to any one of claims 1 to 72, 113 to 119, or 164 to 166 comprising an additional pharmaceutically active agent.

168. The topical composition according to any one of claims 1 to 72, 113 to 119, or 164 to 167, wherein the topical composition does not comprise a barrier compound, in particular not a barrier compound selected from 2-hydroxy-4-methoxybenzophenone and/or 1-phenyl-3-(4′-isopropylphenyl)propane-1,3-dione.

169. The topical composition according to any one of claims 1 to 72, 113 to 119, or 164 to 168 for use in treating or preventing skin irritation.

170. The topical composition according to any one of claims 1 to 72, 113 to 119, or 164 to 169 for use in treating or preventing neurodermitis.

171. The topical composition according to any one of claims 1 to 72, 113 to 119, or 164 to 170 for use in treating or preventing skin sensations preferably selected from the group consisting of stinging, burning, tingling, itching, tickling, skin tightness or any combinations of the foregoing.

172. The topical composition according to any one of claims 1 to 72, 113 to 119, or 164 to 171 for use in treating or preventing skin redness.

173. The topical composition according to any one of claims 1 to 72, 113 to 119, or 164 to 172 for use in
a) treating or preventing skin irritation,
b) treating or preventing neurodermitis,
c) treating or preventing skin sensations selected from the group consisting of stinging, burning, tingling, itching, tickling, skin tightness or any combinations of the foregoing, and/or
d) treating or preventing skin redness.

174. Method for treating or preventing skin irritation comprising contacting the topical composition according to any one of claims 1 to 72, 113 to 119, or 164 to 168 with skin.

175. Method for treating or preventing neurodermitis, comprising contacting the topical composition according to any one of claims 1 to 72, 113 to 119, or 164 to 168 with skin.

176. Method for treating or preventing skin sensations preferably selected from the group consisting of stinging, burning, tingling, itching, tickling, skin tightness or any combinations of the foregoing, comprising contacting the topical composition according to any one of claims 1 to 72, 113 to 119, or 164 to 168 with skin.

177. Method for treating or preventing skin redness, comprising contacting the topical composition according to any one of claims 1 to 72, 113 to 119, or 164 to 168 with skin.

178. Method for
a) treating or preventing skin irritation,
b) treating or preventing neurodermitis,
c) treating or preventing skin sensations selected from the group consisting of stinging, burning, tingling, itching, tickling, skin tightness or any combinations of the foregoing, and/or
d) treating or preventing skin redness.
comprising contacting the topical composition according to any one of claims 1 to 72, 113 to 119, or 164 to 168 with skin.
